(19) United States
(12) Patent Application Publication
(10) Pub. No.: US 2022/0233529 A1
(43) Pub. Date: Jul. 28, 2022

(54) COMBINATION THERAPY COMPRISING A2A/A2B INHIBITORS, PD-1/PD-L1 INHIBITORS, AND ANTI-CD73 ANTIBODIES

(71) Applicant: Incyte Corporation, Wilmington, DE (US)

(72) Inventors: Hui Wang, Hockessin, DE (US); Juan Carlos Almagro, Cambridge, MA (US); Rebecca A. Buonpane, Wilmington, DE (US); Peter Niels Carlsen, Claymont, DE (US); Taisheng Huang, Wilmington, DE (US); Yong Li, Newark, DE (US); Horacio G. Nastri, West Chester, PA (US); Chao Qi, Newark, DE (US); Shaun M. Stewart, Chadds Ford, PA (US); Xiaozhao Wang, Mt. Laurel, NJ (US); Liangxing Wu, Wilmington, DE (US); Wenqing Yao, Chadds Ford, PA (US); Jing Zhou, Boxborough, MA (US); Wenyu Zhu, Media, PA (US)

(21) Appl. No.: 17/564,549

(22) Filed: Dec. 29, 2021

Related U.S. Application Data
(60) Provisional application No. 63/131,659, filed on Dec. 29, 2020.

(51) Int. Cl. A61K 31/501; A61P 35/00; A61K 39/395; C07K 16/28

(52) U.S. Cl. CPC A61K 31/501; A61P 35/00; A61K 39/3955; A61K 2039/545; C07K 16/2818; C07K 16/2896; C07K 16/2827

(57) ABSTRACT

Disclosed are combination therapies comprising administration of a CD73 inhibitor, an adenosine A2A or A2B receptor inhibitor, and a PD-1/PD-L1 inhibitor. The disclosed combination therapies are useful in the treatment of diseases related to the activity of adenosine receptors and/or CD73 and/or PD-1/PD-L1 including, for example, cancer, inflammatory diseases, cardiovascular diseases, and neurodegenerative diseases. Anti-CD73 antibodies, PD-1/PD-L1 inhibitors, and A2A/A2B inhibitors are also disclosed.

### TECHNICAL FIELD

Disclosed herein are combination therapies comprising an inhibitor of A2A/A2B, an inhibitor of PD-1/PD-L1, an anti-CD73 antibody, and methods of using the same to treat disorders such as cancer.

### BACKGROUND

Cluster of differentiation 73 (CD73) is a glycosyl phosphatidyl inositol- (GPI-) linked membrane protein that catalyzes the conversion of extracellular adenosine monophosphate (AMP) to adenosine. It functions as a homodimer, and can be shed and is active as a soluble protein in circulation. In addition to its enzymatic function, CD73 also is a cellular adhesion molecule and plays a role in regulation of leukocyte trafficking. CD73 levels are known to be upregulated due to tissue injury or hypoxic conditions, and a number of solid tumors have elevated CD73 levels. Upregulation of CD73 within the tumor contributes to the adenosine-rich tumor microenvironment, which has numerous pro-tumor and immuno-suppressive effects.

Adenosine is an extracellular signaling molecule that can modulate immune responses through many immune cell types. Adenosine was first recognized as a physiologic regulator of coronary vascular tone by Drury and Szent-Györgyu (Sachdeva, S. and Gupta, M. Saudi Pharmaceutical Journal, 2013, 21, 245-253), however it was not until 1970 that Sattin and Rall showed that adenosine regulates cell function via occupancy of specific receptors on the cell surface (Sattin, A., and Rall, T. W., 1970. Mol. Pharmacol. 6, 13-23; Haskó, G., at al., 2007, Pharmacol. Ther. 113, 264-275).

Adenosine plays a vital role in various other physiological functions. It is involved in the synthesis of nucleic acids, when linked to three phosphate groups; it forms ATP, the integral component of the cellular energy system. Adenosine can be generated by the enzymatic breakdown of extracellular ATP, or can be also released from injured neurons and glial cells by passing the damaged plasma membrane (Tautenhahn, M. et al. Neuropharmacology, 2012, 62, 1756-1766). Adenosine produces various pharmacological effects, both in periphery and in the central nervous system, through an action on specific receptors localized on cell membranes (Matsumoto, T. et al. Pharmacol. Res., 2012, 65, 81-90). Alternative pathways for extracellular adenosine generation have been described. These pathways include the production of adenosine from nicotinamide dinucleotide (NAD) instead of ATP by the concerted action of CD38, CD203a and CD73. CD73-independent production of adenosine can also occur by other phosphates such as alkaline phosphatase or prostate-specific phosphatase.

There are four known subtypes of adenosine receptor in humans including A1, A2A, A2B and A3 receptors. A1 and A2A are high affinity receptors, whereas A2B and A3 are low affinity receptors. Adenosine and its agonists can act via one or more of these receptors and can modulate the activity of adenylate cyclase, the enzyme responsible for increasing cyclic AMP (cAMP). The different receptors have differential stimulatory and inhibitory effects on this enzyme. Increased intracellular concentrations of cAMP can suppress the activity of immune and inflammatory cells (Livingston, M. et al., Inflamm. Res., 2004, 53, 171-178).

The A2A adenosine receptor can signal in the periphery and the CNS, with agonists explored as anti-inflammatory drugs and antagonists explored for neurodegenerative diseases (Carlsson, J. et al., J. Med. Chem., 2010, 53, 3748-3755). In most cell types the A2A subtype inhibits intracellular calcium levels whereas the A2B potentiates them. The A2A receptor generally appears to inhibit inflammatory response from immune cells (Borrmann, T. et al., J. Med. Chem., 2009, 52(13), 3994-4006).

A2B receptors are highly expressed in the gastrointestinal tract, bladder, lung and on mast cells (Antonioli, L. et al., Nature Reviews Cancer, 2013, 13, 842-857). The A2B receptor, although structurally closely related to the A2A receptor and able to activate adenylate cyclase, is functionally different. It has been postulated that this subtype may utilize signal transduction systems other than adenylate cyclase (Livingston, M. et al., Inflamm. Res., 2004, 53, 171-178). Among all the adenosine receptors, the A2B adenosine receptor is a low affinity receptor that is thought to remain silent under physiological conditions and to be activated in consequence of increased extracellular adenosine levels (Ryzhov, S. et al. Neoplasia, 2008, 10, 987-995). Activation of A2B adenosine receptor can stimulate adenylate cyclase and phospholipase C through activation of Gs and Gq proteins, respectively. Coupling to mitogen activated protein kinases has also been described (Borrmann, T. et al., J. Med. Chem., 2009, 52(13), 3994-4006).

In the immune system, engagement of adenosine signaling can be a critical regulatory mechanism that protects tissues against excessive immune reactions. Adenosine can negatively modulate immune responses through many immune cell types, including T-cells, natural-killer cells, macrophages, dendritic cells, mast cells and myeloid-derived suppressor cells (Allard, B. et al. Current Opinion in Pharmacology, 2016, 29, 7-16).

In tumors, this pathway is hijacked by the tumor micro-environment and sabotages the antitumor capacity of the immune system, promoting cancer progression. In the tumor micro-environment, adenosine is mainly generated from extracellular ATP by two ectonucleotidases CD39 and CD73. Multiple cell types can generate adenosine by expressing CD39 and CD73. This is the case for tumor cells, T-effector cells, T-regulatory cells, tumor associated macrophages, myeloid derived suppressive cells (MDSCs), endothelial cells, cancer-associated fibroblast (CAFs) and mesenchymal stromal/stem cells (MSCs). Additionally, hypoxia and inflammation, conditions common to the tumor micro-environment induces expression of CD39 and CD73, leading to increased adenosine production. As a result, the adenosine level in solid tumors is higher compared to normal physiological conditions.

A2A are mostly expressed on lymphoid-derived cells, including T-effector cells, T regulatory cells and natural killer (NK) cells. Blocking A2A receptor can prevent downstream immunosuppressive signals that temporarily inactivate T cells. A2B receptors are mainly expressed on monocyte-derived cells including dendritic cells, tumor-associated macrophages, myeloid derived suppressive cells (MDSCs), and mesenchymal stromal/stem cells (MSCs). Blocking A2B receptor in preclinical models can suppress tumor growth, block metastasis, and increase the presentation of tumor antigens.

In terms of safety profile of ADORA2A/ADORA2B (A2A/A2B) blockage, the A2A and A2B receptor knockout (KO) mice are all viable, showing no growth abnormalities and are fertile (Allard, B. et al. Current Opinion in Pharmacology, 2016, 29, 7-16). A2A KO mice displayed increased levels of pro-inflammatory cytokines only upon challenge with lipopolysaccharides (LPS) and no evidence of inflammation at baseline (Antonioli, L. et al., Nature Reviews Cancer, 2013, 13, 842-857). A2B KO mice exhibited normal platelet, red blood, and white blood cell counts but increased inflammation at baseline such as TNF-alpha and IL-6)(Antonioli, L. et al., Nature Reviews Cancer, 2013, 13, 842-857). Further increase in production of TNF-alpha and IL-6 was detected following LPS treatment. A2B KO mice also exhibited increased vascular adhesion molecules that mediate inflammation as well leukocyte adhesion/rolling; enhanced mast-cell activation; increased sensitivity to IgE-mediated anaphylaxis and increased vascular leakage and neutrophil influx under hypoxia (Antonioli, L. et al., Nature Reviews Cancer, 2013, 13, 842-857).

Some cancer patients have poor long-term prognosis and/or are resistant to one or more types of treatment commonly used in the art. Therefore, a need remains for effective therapies for cancer with increased efficacy and improved safety profiles in this difficult-to-treat patient population.

### SUMMARY

The present application provides, inter alia, a method of treating cancer in a subject, comprising administering to the subject:
(i) an inhibitor of A2A/A2B;
(ii) an inhibitor of PD-1/PD-L1; and
(iii) an inhibitor of human CD73.

The present application further provides methods of treating cancer in a subject, comprising administering to the subject:
(i) an inhibitor of PD-1/PD-L1; and
(ii) an inhibitor of human CD73.

### DESCRIPTION OF DRAWINGS

FIG. 1 shows tumor growth inhibition (TGI) in humanized murine host, carrying the human breast adenocarcinoma tumor, MDA-MB-231, using the treatments described in Example 1.

FIG. 2 shows survival analysis of humanized murine host, carrying the human breast adenocarcinoma tumor, MDA-MB-231, using the treatments described in Example 1.

### DETAILED DESCRIPTION

The present application provides a method of treating cancer in a subject, comprising administering to the subject:

(i) an inhibitor of A2A/A2B;

(ii) an inhibitor of PD-1/PD-L1; and

(iii) an inhibitor of human CD73.

The present application further provides a method of treating cancer in a subject, comprising administering to the subject:

(i) an inhibitor of PD-1/PD-L1; and

(ii) an inhibitor of human CD73.

### A2A/A2B Adenosine Receptor Inhibitors

Adenosine pathway is a critical immune suppressive pathway that protects tissues against excessive immune reactions (Antonioli, L. et al. Nature Review Cancer. 2013, 13, 842-857; Inflamm. Res. 2004, 53: 171-178; Allard, et al. Current Opinion in Pharmacology 2016, 29:7). The immunosuppressive activity of adenosine is mediated through two G-protein coupled receptors (GPCRs) known as A2A and A2B; both receptors are found expressed on many immune cell types, including T-cells, natural-killer cells, macrophages, dendritic cells, mast cells and myeloid-derived suppressor cells (Saudi Pharmaceutical Journal. 2013, 21:245; Frontiers in Immunology. 2019, 10:925; J Clin Invest. 2017, 127(3):929; Neoplasia. 2008, 10: 987; Neoplasia. 2013, 15:1400). As a consequence of the high levels of adenosine production observed in the tumor microenvironment, it has been reported that the antitumor capacity of the immune system is suppressed resulting in cancer progression.

An exemplary amino acid sequence of human A2A adenosine receptor protein (GenBank Accession No. NP_001265428) is:

(SEQ ID NO: 94)
MPIMGSSVYITVELAIAVLAILGNVLVCWAVWLNS
NLQNVTNYFVVSLAAADIAVGVLAIPFAITISTGF
CAACHGCLFIACFVLVLTQSSIFSLLAIAIDRYIA
IRIPLRYNGLVTGTRAKGIIAICWVLSFAIGLTPM
LGWNNCGQPKEGKNHSQGCGEGQVACLFEDVVPMN
YMVYFNFFACVLVPLLLMLGVYLRIFLAARRQLKQ
MESQPLPGERARSTLQKEVHAAKSLAIIVGLFALC
WLPLHIINCFTFFCPDCSHAPLWLMYLAIVLSHTN
SVVNPFIYAYRIREFRQTFRKIIRSHVLRQQEPFK
AAGTSARVLAAHGSDGEQVSLRLNGHPPGVWANGS
APHPERRPNGYALGLVSGGSAQESQGNTGLPDVEL
LSHELKGVCPEPPGLDDPLAQDGAGVS.

An exemplary amino acid sequence of human A2B adenosine receptor protein (GenBank Accession No. NP_000667) is:

(SEQ ID NO: 95)
MLLETQDALYVALELVIAALSVAGNVLVCAAVGTA
NTLQTPTNYFLVSLAAADVAVGLFAIPFAITISLG
FCTDFYGCLFLACFVLVLTQSSIFSLLAVAVDRYL
AICVPLRYKSLVTGTRARGVIAVLWVLAFGIGLTP
FLGWNSKDSATNNCTEPWDGTTNESCCLVKCLFEN
VVPMSYMVYFNFFGCVLPPLLIMLVIYIKIFLVAC
RQLQRTELMDHSRTTLQREIHAAKSLAMIVGIFAL
CWLPVHAVNCVTLFQPAQGKNKPKWAMNMAILLSH
ANSVVNPIVYAYRNRDFRYTFHKIISRYLLCQADV
KSGNGQAGVQPALGVGL.

In some embodiments, the inhibitor of A2A/A2B is a compound selected from Table 1, or a pharmaceutically acceptable salt thereof.

TABLE 1

| Comp. | | |
|---|---|---|
| No. | Name | Structure |
| 1 | 3-(5-Amino-2-(pyridin-2-ylmethyl)-8- (pyrimidin-4-yl)-[1,2,4]triazolo[1,5- c]pyrimidin-7-yl)benzonitrile | 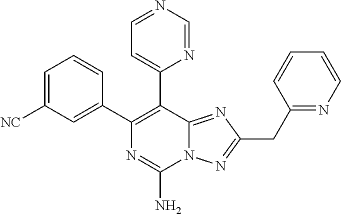 [SMILES: N#Cc1cccc(-c2nc(N)n3nc(Cc4ccccn4)nc3c2-c2ccncn2)c1] |
| 2 | 3-(5-Amino-2-((2,6- difluorophenyl)(hydroxy)methyl)-8- (pyrimidin-4-yl)-[1,2,4]triazolo[1,5- c]pyrimidin-7-yl)benzonitrile | 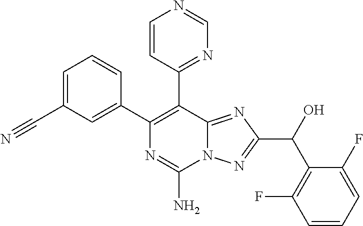 [SMILES: N#Cc1cccc(-c2nc(N)n3nc(C(O)c4c(F)cccc4F)nc3c2-c2ccncn2)c1] |
| 3A | 3-(5-Amino-2-((5-(pyridin-2-yl)-2H- tetrazol-2-yl)methyl)-8-(pyrimidin-4-yl)- [1,2,4]triazolo[1,5-c]pyrimidin-7- yl)benzonitrile | 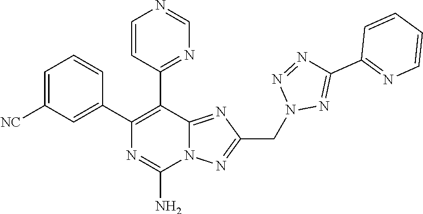 [SMILES: N#Cc1cccc(-c2nc(N)n3nc(Cn4nnc(-c5ccccn5)n4)nc3c2-c2ccncn2)c1] |
| 3B | 3-(5-Amino-2-((5-(pyridin-2-yl)-1H- tetrazol-1-yl)methyl)-8-(pyrimidin-4-yl)- [1,2,4]triazolo[1,5-c]pyrimidin-7- yl)benzonitrile | 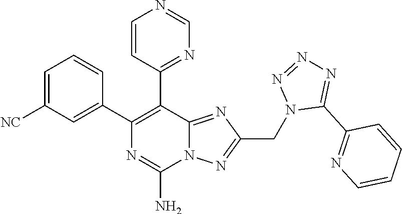 [SMILES: N#Cc1cccc(-c2nc(N)n3nc(Cn4nnnc4-c4ccccn4)nc3c2-c2ccncn2)c1] |
| 4 | 3-(5-Amino-2-((3-methylpyridin-2- yl)methoxy)-8-(pyrimidin-4-yl)- [1,2,4]triazolo[1,5-c]pyrimidin-7- yl)benzonitrile | 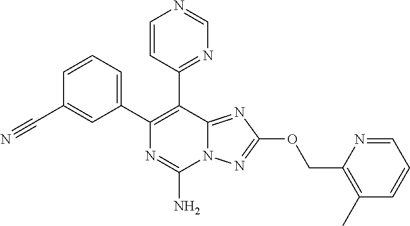 [SMILES: Cc1cccnc1COc1nc2c(-c3ccncn3)c(-c3cccc(C#N)c3)nc(N)n2n1] |
| 5 | 3-(5-Amino-2-(hydroxy(phenyl)methyl)- [1,2,4]triazolo[1,5-c]pyrimidin-7- yl)benzonitrile | 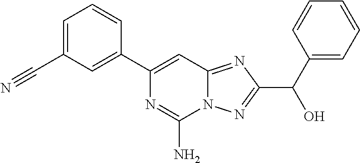 [SMILES: N#Cc1cccc(-c2cc3nc(C(O)c4ccccc4)nn3c(N)n2)c1] |
| 6 | 3-(5-Amino-2-((2,6- difluorophenyl)(hydroxy)methyl)- [1,2,4]triazolo[1,5-c]pyrimidin-7-yl)-2- fluorobenzonitrile | 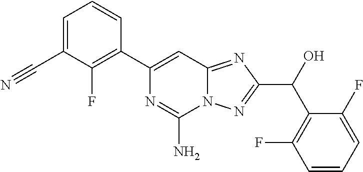 [SMILES: N#Cc1cccc(-c2cc3nc(C(O)c4c(F)cccc4F)nn3c(N)n2)c1F] |
| 7 | 5-Amino-7-(3-cyano-2-fluorophenyl)-2- ((2,6-difluorophenyl)(hydroxy)methyl)- [1,2,4]triazolo[1,5-c]pyrimidine-8- carbonitrile | 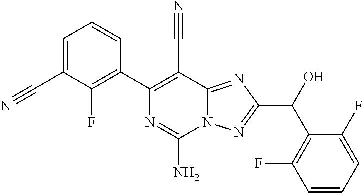 [SMILES: N#Cc1cccc(-c2nc(N)n3nc(C(O)c4c(F)cccc4F)nc3c2C#N)c1F] |
| 8 | 3-(5-Amino-2-((2-fluoro-6-(((1-methyl-2- oxopyrrolidin-3- yl)amino)methyl)phenyl)(hydroxy)methyl)- [1,2,4]triazolo[1,5-c]pyrimidin-7-yl)-2- fluorobenzonitrile | 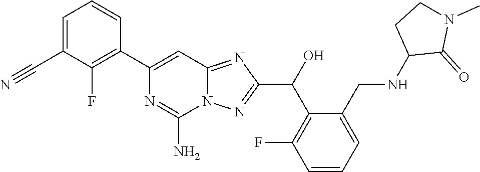 [SMILES: CN1CCC(NCc2cccc(F)c2C(O)c2nc3cc(-c4cccc(C#N)c4F)nc(N)n3n2)C1=O] |
| 9 | 3-(8-Amino-5-(1-methyl-6-oxo-1,6- dihydropyridazin-3-yl)-2-(pyridin-2- ylmethyl)-[1,2,4]triazolo[1,5-c]pyrazin-6- yl)benzonitrile | 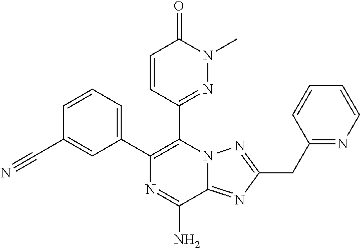 [SMILES: Cn1nc(-c2c(-c3cccc(C#N)c3)nc(N)c3nc(Cc4ccccn4)nn23)ccc1=O] |
| 10 | 3-(8-Amino-2-((2,6- difluorophenyl)(hydroxy)methyl)-5- (pyrimidin-4-yl)-[1,2,4]triazolo[1,5- a]pyrazin-6-yl)benzonitrile | 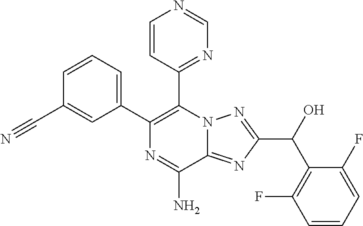 [SMILES: N#Cc1cccc(-c2nc(N)c3nc(C(O)c4c(F)cccc4F)nn3c2-c2ccncn2)c1] |
| 11 | 3-(8-amino-2-(amino(2,6- difluorophenyl)methyl)-5-(4- methyloxazol-5-yl)-[1,2,4]triazolo[1,5- a]pyrazin-6-yl)benzonitrile | 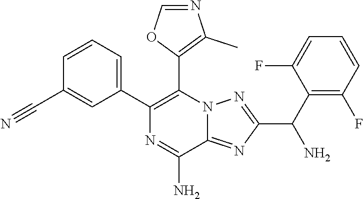 [SMILES: Cc1ncoc1-c1c(-c2cccc(C#N)c2)nc(N)c2nc(C(N)c3c(F)cccc3F)nn12] |
| 12 | 3-(8-amino-2-((2,6- difluorophenyl)(hydroxy)methyl)-5-(2,6- dimethylpyridin-4-yl)-[1,2,4]triazolo[1,5- a]pyrazin-6-yl)benzonitrile | 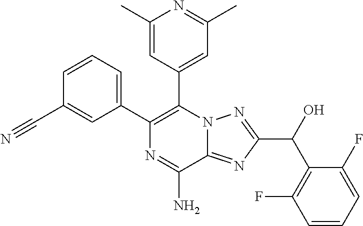 [SMILES: Cc1cc(-c2c(-c3cccc(C#N)c3)nc(N)c3nc(C(O)c4c(F)cccc4F)nn23)cc(C)n1] |
| 13 | 3-(4-amino-2-(pyridin-2-ylmethyl)-7- (pyrimidin-4-yl)-2H-[1,2,3]triazolo[4,5- c]pyridin-6-yl)benzonitrile | 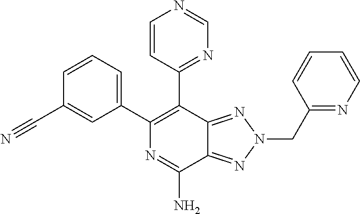 [SMILES: N#Cc1cccc(-c2nc(N)c3nn(Cc4ccccn4)nc3c2-c2ccncn2)c1] |
| 14 | 3-(4-amino-2-((3-fluoropyridin-2- yl)methyl)-7-(pyrimidin-4-yl)-2H- [1,2,3]triazolo[4,5-c]pyridin-6- yl)benzonitrile | 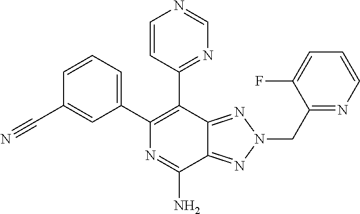 [SMILES: N#Cc1cccc(-c2nc(N)c3nn(Cc4ncccc4F)nc3c2-c2ccncn2)c1] |
| 15 | 3-(4-amino-2-((3-fluoropyridin-2- yl)methyl)-7-(pyridin-4-yl)-2H- [1,2,3]triazolo[4,5-c]pyridin-6- yl)benzonitrile | 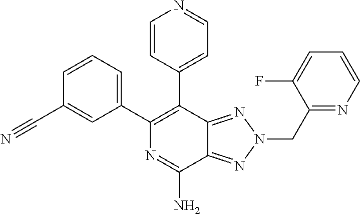 [SMILES: N#Cc1cccc(-c2nc(N)c3nn(Cc4ncccc4F)nc3c2-c2ccncc2)c1] |
| 16 | 3-(4-amino-7-(1-methyl-1H-pyrazol-5- yl)-2-(pyridin-2-ylmethyl)-2H- [1,2,3]triazolo[4,5-c]pyridin-6-yl)-2- fluorobenzonitrile | 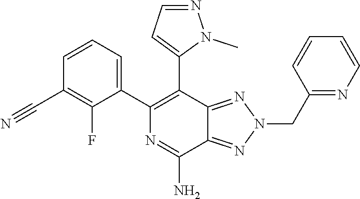 [SMILES: Cn1nccc1-c1c(-c2cccc(C#N)c2F)nc(N)c2nn(Cc3ccccn3)nc12] |
| 17 | 7-(1-((5-Chloropyridin-3-yl)methyl)-1H- pyrazol-4-yl)-3-methyl-9-pentyl-6,9- dihydro-5H-pyrrolo[3,2- d][1,2,4]triazolo[4,3-a]pyrimidin-5-one | 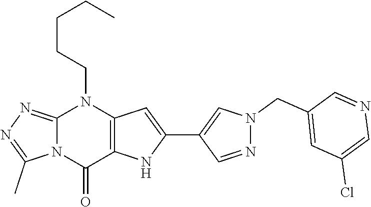 [SMILES: CCCCCn1c2cc(-c3cnn(Cc4cncc(Cl)c4)c3)[nH]c2c(=O)n2c(C)nnc12] |
| 18 | 3-Methyl-7-(1-((5-methylpyridin-3- yl)methyl)-1H-pyrazol-4-yl)-9-pentyl-6,9- dihydro-5H-pyrrolo[3,2- d][1,2,4]triazolo[4,3-a]pyrimidin-5-one | 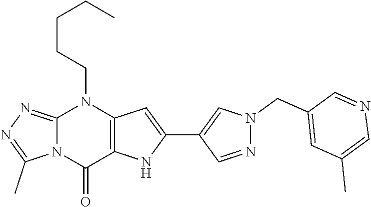 [SMILES: CCCCCn1c2cc(-c3cnn(Cc4cncc(C)c4)c3)[nH]c2c(=O)n2c(C)nnc12] |
| 19 | 3-Methyl-9-pentyl-7-(1-(thieno[3,2- b]pyridin-6-ylmethyl)-1H-pyrazol-4-yl)- 6,9-dihydro-5H-pyrrolo[3,2- d][1,2,4]triazolo[4,3-a]pyrimidin-5-one | 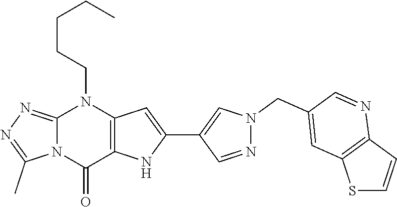 [SMILES: CCCCCn1c2cc(-c3cnn(Cc4cnc5ccsc5c4)c3)[nH]c2c(=O)n2c(C)nnc12] |
| 20 | 7-(1-((2-(2-(Dimethylamino)acetyl)- 1,2,3,4-tetrahydroisoquinolin-6- yl)methyl)-1H-pyrazol-4-yl)-3-methyl-9- pentyl-6,9-dihydro-5H-pyrrolo[3,2- d][1,2,4]triazolo[4,3-a]pyrimidin-5-one | 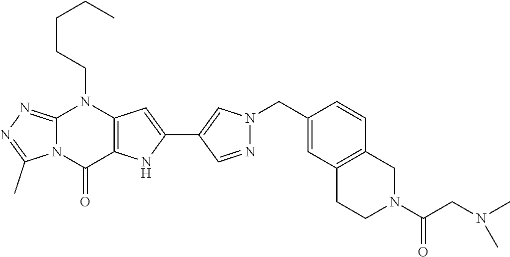 [SMILES: CCCCCn1c2cc(-c3cnn(Cc4ccc5c(c4)CCN(C(=O)CN(C)C)C5)c3)[nH]c2c(=O)n2c(C)nnc12] |
| 21A | 3-(2-((5-(1H-Pyrazol-1-yl)-2H-tetrazol-2- yl)methyl)-5-amino-8-(pyrimidin-4-yl)- [1,2,4]triazolo[1,5-c]pyrimidin-7- yl)benzonitrile | 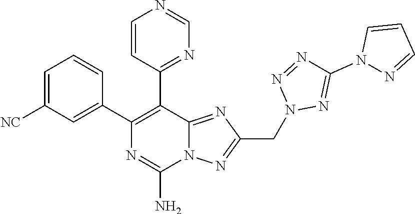 [SMILES: N#Cc1cccc(-c2nc(N)n3nc(Cn4nnc(-n5cccn5)n4)nc3c2-c2ccncn2)c1] |
| 21B | 3-(2-((5-(1H-Pyrazol-1-yl)-1H-tetrazol-1- yl)methyl)-5-amino-8-(pyrimidin-4-yl)- [1,2,4]triazolo[1,5-c]pyrimidin-7- yl)benzonitrile | 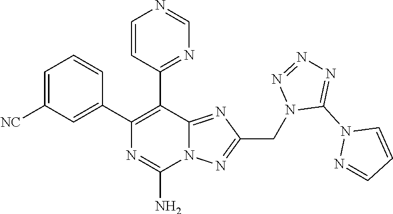 [SMILES: N#Cc1cccc(-c2nc(N)n3nc(Cn4nnnc4-n4cccn4)nc3c2-c2ccncn2)c1] |

In some embodiments, the inhibitor of A2A/A2B is a compound of Formula (I):

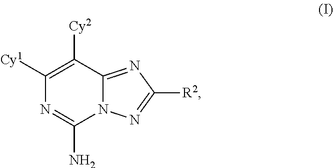

or a pharmaceutically acceptable salt thereof, wherein

Cy¹ is phenyl which is substituted by 1 or 2 substituents independently selected from halo and CN;

Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;

R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl- of R² are each optionally substituted with 1, 2, or 3 independently selected R^{C} substituents;

R^{a2} is (5-7 membered heteroaryl)-C₁₋₃ alkyl- optionally substituted with 1 or 2 independently selected R^{C} substituents;

each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and

each R^{a4}, R^{c4}, and R^{d4} are independently selected from H and C₁₋₆ alkyl.

In some embodiments of the compound of Formula (I), Cy² is pyrimidinyl.

In some embodiments of the compound of Formula (I), R² is selected from pyridin-2-ylmethyl, (2,6-difluorophenyl)(hydroxy)methyl, (5-(pyridin-2-yl)-1H-tetrazol-1-yl)methyl, (3-methylpyridin-2-yl)methoxy, and (5-(1H-Pyrazol-1-yl)-1H-tetrazol-1-yl)methyl.

In some embodiments, the compound of Formula (I), or a pharmaceutically acceptable salt thereof, is 3-(5-Amino-2-(pyridin-2-ylmethyl)-8-(pyrimidin-4-yl)-[1,2,4]triazolo[1,5-c]pyrimidin-7-yl)benzonitrile, or a pharmaceutically acceptable salt thereof (see Compound 1, Table 1).

In some embodiments, the compound of Formula (I), or a pharmaceutically acceptable salt thereof, is 3-(5-Amino-2-((2,6-difluorophenyl)(hydroxy)methyl)-8-(pyrimidin-4-yl)-[1,2,4]triazolo[1,5-c]pyrimidin-7-yl)benzonitrile, or a pharmaceutically acceptable salt thereof (see Compound 2, Table 1).

In some embodiments, the compound of Formula (I), or a pharmaceutically acceptable salt thereof, is 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof (see Compound 3A, Table 1).

In some embodiments, the compound of Formula (I), or a pharmaceutically acceptable salt thereof, is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof (see Compound 3B, Table 1).

In some embodiments, the compound of Formula (I), or a pharmaceutically acceptable salt thereof, is 3-(5-Amino-2-((3-methylpyridin-2-yl)methoxy)-8-(pyrimidin-4-yl)-[1,2,4]triazolo[1,5-c]pyrimidin-7-yl)benzonitrile, or a pharmaceutically acceptable salt thereof (see Compound 4, Table 1).

In some embodiments, the compound of Formula (I), or a pharmaceutically acceptable salt thereof, is 3-(2-((5-(1H-Pyrazol-1-yl)-2H-tetrazol-2-yl)methyl)-5-amino-8-(pyrimidin-4-yl)-[1,2,4]triazolo[1,5-c]pyrimidin-7-yl)benzonitrile, or a pharmaceutically acceptable salt thereof (see Compound 21A, Table 1).

In some embodiments, the compound of Formula (I), or a pharmaceutically acceptable salt thereof, is 3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile, or a pharmaceutically acceptable salt thereof (see Compound 21B, Table 1).

The synthesis and characterization of compounds of Formula (I) can be found in WO2019/168847 and U.S. 62/891,685, both of which are hereby incorporated by reference in their entireties.

In some embodiments, the inhibitor of A2A/A2B is a compound of Formula (II):

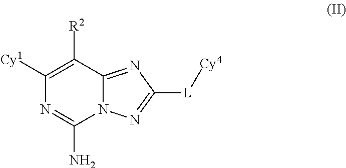

or a pharmaceutically acceptable salt thereof, wherein

R² is selected from H and CN;

Cy¹ is phenyl which is substituted by 1 or 2 substituents independently selected from halo and CN;

L is C₁₋₃ alkylene, wherein said alkylene is optionally substituted with 1, 2, or 3 independently selected RD substituents;

Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;

each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;

each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₆ alkyl, 5-6 membered heteroaryl, and 4-7 membered heterocycloalkyl of R^{8A} are each optionally substituted with 1, 2, or 3 independently selected R^{8B} substituents;

each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R8^{B} substituents;

each R^{8B} is independently selected from halo and C₁₋₃ alkyl; and

each R^{8D} is independently selected from OH, CN, halo, C₁₋₆ alkyl, and C₁₋₆ haloalkyl.

In some embodiments, the compound of Formula (II), or a pharmaceutically acceptable salt thereof, is 3-(5-Amino-2-(hydroxy(phenyl)methyl)-[1,2,4]triazolo[1,5-c]pyrimidin-7-yl)benzonitrile, or a pharmaceutically acceptable salt thereof (see Compound 5, Table 1).

In some embodiments, the compound of Formula (II), or a pharmaceutically acceptable salt thereof, is 3-(5-Amino-2-((2,6-difluorophenyl)(hydroxy)methyl)-[1,2,4]triazolo[1,5-c]pyrimidin-7-yl)-2-fluorobenzonitrile, or a pharmaceutically acceptable salt thereof (see Compound 6, Table 1).

In some embodiments, the compound of Formula (II), or a pharmaceutically acceptable salt thereof, is 5-Amino-7-(3-cyano-2-fluorophenyl)-2-((2,6-difluorophenyl)(hydroxy)methyl)-[1,2,4]triazolo[1,5-c]pyrimidine-8-carbonitrile, or a pharmaceutically acceptable salt thereof (see Compound 7, Table 1).

In some embodiments, the compound of Formula (II), or a pharmaceutically acceptable salt thereof, is 3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile, or a pharmaceutically acceptable salt thereof (see Compound 8, Table 1).

The synthesis and characterization of compounds of Formula (II) can be found in WO2019/222677, which is hereby incorporated by reference in its entirety.

In some embodiments, the inhibitor of A2A/A2B is a compound of Formula (III):

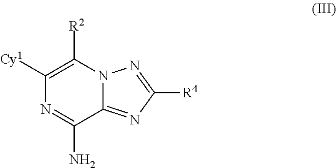

or a pharmaceutically acceptable salt thereof, wherein

Cy¹ is phenyl which is substituted by 1 or 2 substituents independently selected from halo and CN;

R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;

each R^{2A} is independently selected from D, halo, C₁₋₆ alkyl, and C₁₋₆ haloalkyl; R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and

each R^{a41}, R^{c41}, and R^{d41} is independently selected from H and C₁₋₆ alkyl.

In some embodiments, the compound of Formula (III), or a pharmaceutically acceptable salt thereof, is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof (see Compound 9, Table 1).

In some embodiments, the compound of Formula (III), or a pharmaceutically acceptable salt thereof, is 3-(8-Amino-2-((2,6-difluorophenyl)(hydroxy)methyl)-5-(pyrimidin-4-yl)-[1,2,4]triazolo[1,5-a]pyrazin-6-yl)benzonitrile, or a pharmaceutically acceptable salt thereof (See Compound 10, Table 1).

In some embodiments, the compound of Formula (III), or a pharmaceutically acceptable salt thereof, is 3-(8-amino-2-(amino(2,6-difluorophenyl)methyl)-5-(4-methyloxazol-5-yl)-[1,2,4]triazolo[1,5-a]pyrazin-6-yl)benzonitrile, or a pharmaceutically acceptable salt thereof (see Compound 11, Table 1).

In some embodiments, the compound of Formula (III), or a pharmaceutically acceptable salt thereof, is 3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile, or a pharmaceutically acceptable salt thereof (see Compound 12, Table 1).

The synthesis and characterization of compounds of Formula (III) can be found in PCT/US2019/040496, which is hereby incorporated by reference in its entirety.

In some embodiments, the inhibitor of A2A/A2B is a compound of Formula (IV):

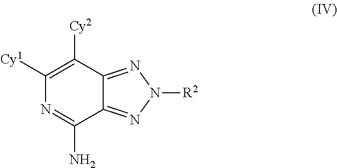

or a pharmaceutically acceptable salt thereof, wherein

Cy¹ is phenyl which is substituted by 1 or 2 substituents independently selected from halo and CN;

Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;

each R⁶ is independently selected from halo, C₁₋₆ alkyl, and C₁₋₆ haloalkyl;

R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and

each R^{2A} is independently selected from halo, C₁₋₆ alkyl, and C₁₋₆ haloalkyl.

In some embodiments, the compound of Formula (IV), or a pharmaceutically acceptable salt thereof, is 3-(4-amino-2-(pyridin-2-ylmethyl)-7-(pyrimidin-4-yl)-2H-[1,2,3]triazolo[4,5-c]pyridin-6-yl)benzonitrile, or a pharmaceutically acceptable salt thereof (see Compound 13, Table 1).

In some embodiments, the compound of Formula (IV), or a pharmaceutically acceptable salt thereof, is 3-(4-amino-2-((3-fluoropyridin-2-yl)methyl)-7-(pyrimidin-4-yl)-2H-[1,2,3]triazolo[4,5-c]pyridin-6-yl)benzonitrile, or a pharmaceutically acceptable salt thereof (see Compound 14, Table 1).

In some embodiments, the compound of Formula (IV), or a pharmaceutically acceptable salt thereof, is 3-(4-amino-2-((3-fluoropyridin-2-yl)methyl)-7-(pyridin-4-yl)-2H-[1,2,3]triazolo[4,5-c]pyridin-6-yl)benzonitrile, or a pharmaceutically acceptable salt thereof (see Compound 15, Table 1).

In come embodiments, the compound of Formula (IV), or a pharmaceutically acceptable salt thereof, is 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile, or a pharmaceutically acceptable salt thereof (see Compound 16, Table 1).

The synthesis and characterization of compounds of Formula (IV) can be found in U.S. 62/798,180, which is hereby incorporated by reference in its entirety.

In some embodiments, the inhibitor of A2A/A2B is a compound of Formula (V):

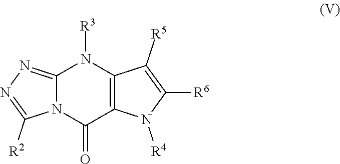

or a pharmaceutically acceptable salt thereof, wherein

R² is selected from H, D, halo, C₁₋₆ alkyl and C₁₋₆ haloalkyl;

R³ is selected from H and C₁₋₆ alkyl;

R⁴ is selected from H and C₁₋₆ alkyl;

R⁵ is selected from H, halo, CN, C₁₋₆ alkyl;

R⁶ is selected from phenyl, C₃₋₇ cycloalkyl, 5-7 membered heteroaryl, and 4-7 membered heterocycloalkyl wherein said phenyl, C₃₋₇ cycloalkyl, 5-7 membered heteroaryl, and 4-7 membered heterocycloalkyl of R⁶ are optionally substituted by 1, 2, or 3 independently selected R^{A} substituents;

each R^{A} is independently selected from (5-10 membered heteroaryl)-C₁₋₃ alkyl- and (4-10 membered heterocycloalkyl)-C₁₋₃ alkyl-, wherein the (5-10 membered heteroaryl)-C₁₋₃ alkyl- and (4-10 membered heterocycloalkyl)-C₁₋₃ alkyl- of R^{A} are each optionally substituted with 1 or 2 independently selected R^{B} substituents;

each R^{B} is independently selected from halo, C₁₋₆ alkyl, and C(O)R^{b26};

R^{b26} is independently selected from H and C₁₋₃ alkyl, wherein the C₁₋₃ alkyl of R^{b26} is optionally substituted with 1 or 2 independently selected R^{C} substituents

each R^{C} is independently selected from halo, C₁₋₆ alkyl, CN, OR^{a36}, and NR^{c36}R^{d36}, and

each R^{a36}, R^{c36}, and R^{d36} is independently selected from H and C₁₋₆ alkyl.

In some embodiments, the compound of Formula (V), or a pharmaceutically acceptable salt thereof, is 7-(1-((5-Chloropyridin-3-yl)methyl)-1H-pyrazol-4-yl)-3-methyl-9-pentyl-6,9-dihydro-5H-pyrrolo[3,2-d][1,2,4]triazolo[4,3-a]pyrimidin-5-one, or a pharmaceutically acceptable salt thereof (see Compound 17, Table 1).

In some embodiments, the compound of Formula (V), or a pharmaceutically acceptable salt thereof, is 3-Methyl-7-(1-((5-methylpyridin-3-yl)methyl)-1H-pyrazol-4-yl)-9-pentyl-6,9-dihydro-5H-pyrrolo[3,2-d][1,2,4]triazolo[4,3-a]pyrimidin-5-one, or a pharmaceutically acceptable salt thereof (see Compound 18, Table 1).

In some embodiments, the compound of Formula (V), or a pharmaceutically acceptable salt thereof, is 3-Methyl-9-pentyl-7-(1-(thieno[3,2-b]pyridin-6-ylmethyl)-1H-pyrazol-4-yl)-6,9-dihydro-5H-pyrrolo[3,2-d][1,2,4]triazolo[4,3-a]pyrimidin-5-one, or a pharmaceutically acceptable salt thereof (see Compound 19, Table 1).

In some embodiments, the compound of Formula (V), or a pharmaceutically acceptable salt thereof, is 7-(1-((2-(2-(Dimethylamino)acetyl)-1,2,3,4-tetrahydroisoquinolin-6-yl)methyl)-1H-pyrazol-4-yl)-3-methyl-9-pentyl-6,9-dihydro-5H-pyrrolo[3,2-d][1,2,4]triazolo[4,3-a]pyrimidin-5-one, or a pharmaceutically acceptable salt thereof (see Compound 20, Table 1).

The synthesis and characterization of compounds of Formula (V) can be found in US-2019-0337957, which is hereby incorporated by reference in its entirety.

Other inhibitors of A2A and/or A2B adenosine receptor useful in the methods described herein are known in the art.

In some instances, the inhibitor of A2A and/or A2B adenosine receptor is CPI-444 (also referred to herein as “Compound B”; 7-(5-methylfuran-2-yl)-3-[[6-[[(3S)-oxolan-3-yl]oxymethyl]pyridin-2-yl]methyl]triazolo[4,5-d]pyrimidin-5-amine).

In some instances, the inhibitor of A2A and/or A2B adenosine receptor is AB928 (3-[2-Amino-6-[1-[[6-(2-hydroxypropan-2-yl)pyridin-2-yl]methyl]triazol-4-yl]pyrimidin-4-yl]-2-methylbenzonitrile).

In some instances, the inhibitor of A2A and/or A2B adenosine receptor is AZD4635 (6-(2-Chloro-6-methylpyridin-4-yl)-5-(4-fluorophenyl)-1,2,4-triazin-3-amine).

In some instances, the inhibitor of A2A and/or A2B adenosine receptor is NIR-178 (5-Bromo-2,6-di(1H-pyrazol-1-yl)pyrimidin-4-amine).

In some instances, the inhibitor of A2A and/or A2B adenosine receptor is EOS100850.

In some instances, the inhibitor of A2A and/or A2B adenosine receptor is a compound, pharmaceutically acceptable salt thereof, or stereoisomer thereof described in US patent application publication no. 2019/0292188, which is incorporated by reference herein in its entirety.

As used herein, “about” when referring to a measurable value such as an amount, a dosage, a temporal duration, and the like, is meant to encompass variations of ±10%. In certain embodiments, “about” can include variations of ±5%, ±1%, or ±0.1% from the specified value and any variations there between, as such variations are appropriate to perform the disclosed methods.

In some embodiments, the compound disclosed herein is the (S)-enantiomer of the compound, or a pharmaceutically acceptable salt thereof. In some embodiments, the compound is the (R)-enantiomer of the compound, or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl.

In some embodiments, the aryl is phenyl (i.e., C₆ aryl).

As used herein, “halo” or “halogen” refers to F, Cl, Br, or I. In some embodiments, a halo is F, Cl, or Br. In some embodiments, a halo is F or Cl. In some embodiments, a halo is F. In some embodiments, a halo is Cl.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃-10). In some embodiments, the cycloalkyl is a C₃-10 monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1, 2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)², etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl and the like. For example, heterocycloalkyl groups include the following groups (with and without N-methyl substitution):

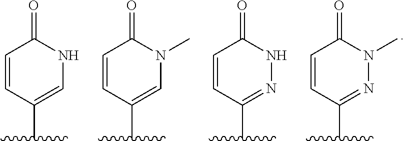

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

As used herein, the term “independently selected from” means that each occurrence of a variable or substituent are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as 0-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

Compounds described herein, including salts thereof, can be prepared using known organic synthesis techniques and can be synthesized according to any of numerous possible synthetic routes.

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures, which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) (“Preparative LC-MS Purification: Improved Compound Specific Method Optimization” Karl F. Blom, et al. J. Combi. Chem. 2004, 6(6), 874-883, which is incorporated herein by reference in its entirety) and normal phase silica chromatography.

The compounds described herein can modulate activity of one or more of various G-protein coupled receptors (GPCRs) including, for example, A2A/A2B. The term “modulate” is meant to refer to an ability to increase or decrease the activity of one or more members of the A2A/A2B family. Accordingly, the compounds described herein can be used in methods of modulating A2A/A2B by contacting the A2A/A2B with any one or more of the compounds or compositions described herein. In some embodiments, compounds of the present invention can act as inhibitors of one or both of A2A and A2B. In further embodiments, the compounds described herein can be used to modulate activity of A2A/A2B in an individual in need of modulation of the receptor by administering a modulating amount of a compound described herein, or a pharmaceutically acceptable salt thereof. In some embodiments, modulating is inhibiting.

Given that cancer cell growth and survival is impacted by multiple signaling pathways, the present invention is useful for treating disease states characterized by drug resistant mutants. In addition, different GPCR inhibitors, exhibiting different preferences in the GPCRs which they modulate the activities of, may be used in combination. This approach could prove highly efficient in treating disease states by targeting multiple signaling pathways, reduce the likelihood of drug-resistance arising in a cell, and reduce the toxicity of treatments for disease.

GPCRs to which the present compounds bind and/or modulate (e.g., inhibit) include any member of the A2A/A2B family.

In some embodiments, more than one compound described herein is used to inhibit the activity of one GPCR (e.g., A2A).

In some embodiments, more than one compound described herein is used to inhibit more than one GPCR, such as at least two GPCRs (e.g., A2A and A2B).

In some embodiments, one or more of the compounds is used in combination with another GPCR antagonist to inhibit the activity of one GPCR (e.g., A2A or A2B).

The inhibitors of A2A/A2B described herein can be selective. By “selective” is meant that the compound binds to or inhibits a GPCR with greater affinity or potency, respectively, compared to at least one other GPCR. In some embodiments, the compounds described herein are selective inhibitors of A2A or A2B. In some embodiments, the compounds described herein are selective inhibitors of A2A (e.g., over A2B). In some embodiments, the compounds described herein are selective inhibitors of A2B (e.g., over A2A). In some embodiments, selectivity can be at least about 2-fold, 5-fold, 10-fold, at least about 20-fold, at least about 50-fold, at least about 100-fold, at least about 200-fold, at least about 500-fold or at least about 1000-fold. Selectivity can be measured by methods routine in the art. In some embodiments, selectivity can be tested at the biochemical affinity against each GPCR. In some embodiments, the selectivity of compounds described herein can be determined by cellular assays associated with particular A2A/A2B GPCR activity.

### PD-1/PD-L1 Inhibitors

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (Postow et al., J Clinical Oncology 2015, 1-9). Blocking the signaling of an inhibitory immune checkpoint, such as PD-1, has proven to be a promising and effective treatment modality.

Programmed Death-1 (“PD-1,” also known as “CD279”) is an approximately 31 kD type I membrane protein member of the extended CD28/CTLA-4 family of T-cell regulators that broadly negatively regulates immune responses (Ishida, Y. et al. (1992) EMBO J 11:3887-3895; United States Patent Publication No. 2007/0202100; 2008/0311117; and 2009/00110667; U.S. Pat. Nos. 6,808,710; 7, 101,550; 7,488,802; 7,635,757; and 7,722,868; PCT Publication No. WO 01/14557).

PD-1 is expressed on activated T-cells, B-cells, and monocytes (Agata, Y. et al. (1996) Int. Immunol. 8(5):765-772; Yamazaki, T. et al. (2002) J. Immunol. 169:5538-5545) and at low levels in natural killer (NK) T-cells (Nishimura, H. et al. (2000) J. Exp. Med. 191:891-898; Martin-Orozco, N. et al. (2007) Semin. Cancer Biol. 17(4):288-298).

The extracellular region of PD-1 consists of a single immunoglobulin (Ig)V domain with 23% identity to the equivalent domain in CTLA-4 (Martin-Orozco, N. et al. (2007) Semin. Cancer Biol. 17(4):288-298). The extracellular IgV domain is followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals (Ishida, Y. et al. (1992) EMBO J. 11:3887-3895; Blank, C. et al. (2006) Immunol. Immunother. 56(5):739-745).

PD-1 mediates its inhibition of the immune system by binding to B7-H1 and B7-DC (Flies, D. B. et al. (2007) J. Immunother. 30(3):251-260; U.S. Pat. Nos. 6,803,192; 7,794,710; United States Patent Application Publication Nos. 2005/0059051; 2009/0055944; 2009/0274666; 2009/0313687; PCT Publication Nos. WO 01/39722; WO 02/086083).

The amino acid sequence of the human PD-1 protein (Genbank Accession No. NP_005009) is:

(SEQ ID NO: 1)
MQIPQAPWPVVWAVLQLGWRPGWFLDSPDRPWNPP
TFSPALLVVTEGDNATFTCSFSNTSESFVLNWYRM
SPSNQTDKLAAFPEDRSQPGQDCRFRVTQLPNGRD
FHMSVVRARRNDSGTYLCGAISLAPKAQIKESLRA
ELRVTERRAEVPTARPSPSPRPAGQFQTLVVGVVG
GLLGSLVLLVWVLAVICSRAARGTIGARRTGQPLK
EDPSAVPVFSVDYGELDFQWREKTPEPPVPCVPEQ
TEYATIVFPSGMGTSSPARRGSADGPRSAQPLRPE
DGHCSWPL.

PD-1 has two ligands, PD-L1 and PD-L2 (Parry et al., Mol Cell Biol 2005, 9543-9553; Latchman et al, Nat Immunol 2001, 2, 261-268), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-γ treatment (Iwai et al., PNAS 2002, 99(19):12293-7; Blank et al., Cancer Res 2004, 64(3):1140-5). In fact, tumor PD-L1 expression status has been shown to be prognostic in multiple tumor types (Wang et al., Eur J Surg Oncol 2015; Huang et al., Oncol Rep 2015; Sabatier et al., Oncotarget 2015, 6(7): 5449-5464). PD-L2 expression, in contrast, is more restricted and is expressed mainly by dendritic cells (Nakae et al, J Immunol 2006, 177:566-73). Ligation of PD-1 with its ligands PD-L1 and PD-L2 on T cells delivers a signal that inhibits IL-2 and IFN-γ production, as well as cell proliferation induced upon T cell receptor activation (Carter et al., Eur J Immunol 2002, 32(3):634-43; Freeman et al, J Exp Med 2000, 192(7):1027-34). The mechanism involves recruitment of SHP-2 or SHP-1 phosphatases to inhibit T cell receptor signaling such as Syk and Lck phosphorylation (Sharpe et al., Nat Immunol 2007, 8, 239-245). Activation of the PD-1 signaling axis also attenuates PKC-θ activation loop phosphorylation, which is necessary for the activation of NF-κB and AP1 pathways, and for cytokine production such as IL-2, IFN-γ and TNF (Sharpe et al., Nat Immunol 2007, 8, 239-245; Carter et al., Eur J Immunol 2002, 32(3):634-43; Freeman et al., J Exp Med 2000, 192(7):1027-34).

Several lines of evidence from preclinical animal studies indicate that PD-1 and its ligands negatively regulate immune responses. PD-1-deficient mice have been shown to develop lupus-like glomerulonephritis and dilated cardiomyopathy (Nishimura et al., Immunity 1999, 11:141-151; Nishimura et al., Science 2001, 291:319-322). Using an LCMV model of chronic infection, it has been shown that PD-1/PD-L1 interaction inhibits activation, expansion and acquisition of effector functions of virus-specific CD8 T cells (Barber et al., Nature 2006, 439, 682-7). Together, these data support the development of a therapeutic approach to block the PD-1-mediated inhibitory signaling cascade in order to augment or “rescue” T cell response. Accordingly, there is a need for new methods of blocking PD-1/PD-L1 protein/protein interaction, and thereby treating cancer in a subject.

In some embodiments, the inhibitor of PD-1/PD-L1 is a compound selected from nivolumab (OPDIVO®, BMS-936558, MDX1106, or MK-34775), pembrolizumab (KEYTRUDA®, MK-3475, SCH-900475, lambrolizumab, CAS Reg. No. 1374853-91-4), atezolizumab (Tecentriq®, CAS Reg. No. 1380723-44-3), durvalumab, avelumab (Bavencio®), cemiplimab, AMP-224, AMP-514/MEDI-0680, atezolizumab, avelumab, BGB-A317, BMS936559, durvalumab, JTX-4014, SHR-1210, pidilizumab (CT-011), REGN2810, BGB-108, BGB-A317, SHR-1210 (HR-301210, SHR1210, or SHR-1210), BMS-936559, MPDL3280A, MEDI4736, MSB0010718C, MDX1105-01, and one or more of the PD-1/PD-L1 blocking agents described in U.S. Pat. Nos. 7,488,802, 7,943,743, 8,008,449, 8,168,757, 8,217,149, or Pub. Nos. WO 03042402, WO 2008/156712, WO 2010/089411, WO 2010/036959, WO 2011/066342, WO 2011/159877, WO 2011/082400, WO 2011/161699, WO 2017/070089, WO 2017/087777, WO 2017/106634, WO 2017/112730, WO 2017/192961, WO 2017/205464, WO 2017/222976, WO 2018/013789, WO 2018/04478, WO 2018/119236, WO 2018/119266, WO 2018/119221, WO 2018/119286, WO 2018/119263, WO 2018/119224, WO 2019/191707, and WO 2019/217821, and any combinations thereof. The disclosure of each of the preceding patents, applications, and publications is incorporated herein by reference in its entirety.

In some embodiments, the inhibitor of PD-1/PD-L1 is selected from a compound as disclosed in WO 2018/119266 such as, e.g.,
(S)-1-((7-chloro-2-(2′-chloro-3′-(5-(((2-hydroxyethyl)amino)methyl)picolinamido)-2-methyl-[1,1′-biphenyl]-3-yl)benzo[d]oxazol-5-yl)methyl)piperidine-2-carboxylic acid, or a pharmaceutically acceptable salt thereof;
(S)-1-((7-chloro-2-(3′-(7-chloro-5-(((S)-3-hydroxypyrrolidin-1-yl)methyl)benzo[d]oxazol-2-yl)-2,2′-dimethylbiphenyl-3-yl)benzo[d]oxazol-5-yl)methyl)pyrrolidine-3-carboxylic acid, or a pharmaceutically acceptable salt thereof;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof;
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid, or a pharmaceutically acceptable salt thereof;
(R)-1-((7-cyano-2-(2,2′-dimethyl-3′-(4,5,6,7-tetrahydrothiazolo[5,4-c]pyridin-2-yl)biphenyl-3-yl)benzo[d]oxazol-5-yl)methyl)pyrrolidine-3-carboxylic acid, or a pharmaceutically acceptable salt thereof;
(R)-1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)pyrrolidine-3-carboxylic acid, or a pharmaceutically acceptable salt thereof, and
1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)piperidine-4-carboxylic acid, or a pharmaceutically acceptable salt thereof.

In some embodiments, the inhibitor of PD-1/PD-L1 is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof. The synthesis and characterization of (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid is disclosed in WO 2018/119266, which is hereby incorporated by reference in its entirety.

In some embodiments, the inhibitor of PD-1/PD-L1 is selected from:
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid hydrobromic acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid oxalic acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid hydrochloric acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid L-tartaric acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid malonic acid salt; and
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid phosphoric acid salt.

In some embodiments, the inhibitor of PD-1/PD-L1 is selected from a compound disclosed in WO 2018/119224 such as, e.g.,
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid, or a pharmaceutically acceptable salt thereof;
(R)-1-((2-(2′-chloro-3′-(6-isopropyl-4,5,6,7-tetrahydro-2H-pyrazolo[3,4-c]pyridin-2-yl)-2-methylbiphenyl-3-yl)-7-cyanobenzo[d]oxazol-5-yl)methyl)pyrrolidine-3-carboxylic acid, or a pharmaceutically acceptable salt thereof;
(S)—N-(2-chloro-3′-(5-(2-hydroxypropyl)-1-methyl-4,5,6,7-tetrahydro-1H-imidazo[4,5-c]pyridine-2-carboxamido)-2′-methylbiphenyl-3-yl)-5-isopropyl-1-methyl-4,5,6,7-tetrahydro-1H-imidazo[4,5-c]pyridine-2-carboxamide, or a pharmaceutically acceptable salt thereof;
cis-4-((2-((2,2′-dichloro-3′-(1-methyl-5-(tetrahydro-2H-pyran-4-yl)-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)methyl)cyclohexane-1-carboxylic acid, or a pharmaceutically acceptable salt thereof;
trans-4-(2-(2-((2,2′-dichloro-3′-(5-(2-hydroxyethyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid, or a pharmaceutically acceptable salt thereof;
trans-4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid, or a pharmaceutically acceptable salt thereof; and
cis-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)methyl)cyclohexane-1-carboxylic acid, or a pharmaceutically acceptable salt thereof.

In some embodiments, the inhibitor of PD-1/PD-L1 is selected from a compound disclosed in WO 2019/191707 such as, e.g.,
(R)-1-((7-cyano-2-(3′-(7-((3-hydroxypyrrolidin-1-yl)methyl)-2-methylpyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof;
(R)-1-((7-cyano-2-(3′-(7-(((S)-1-hydroxypropan-2-ylamino)methyl)-2-methylpyrido[3,2-d]pyrimidin-4-ylamino)-2,2′-dimethylbiphenyl-3-yl)benzo[d]oxazol-5-yl)methyl)pyrrolidine-3-carboxylic acid, or a pharmaceutically acceptable salt thereof;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)-N,N-dimethylpiperidine-4-carboxamide, or a pharmaceutically acceptable salt thereof;
(R)-1-((7-cyano-2-(3′-(2-cyclopropyl-7-(((R)-3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)pyrrolidine-3-carboxylic acid, or a pharmaceutically acceptable salt thereof; and
(R)-1-((7-cyano-2-(3′-(3-(((R)-3-hydroxypyrrolidin-1-yl)methyl)-6-methyl-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.

In some embodiments, the inhibitor of PD-1/PD-L1 is selected from a compound disclosed in WO 2019/217821 such as, e.g.,
4-(2-(2-((2,2′-dichloro-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4-(2-(2-((3′-(5-((1H-pyrazol-3-yl)methyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
(R)-4-(2-(2-((2,2′-dichloro-3′-(5-(2-hydroxypropyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or a pharmaceutically acceptable salt thereof;
4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof,
4-(2-(2-((2,2′-dimethyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof; and
4-(2-(2-((3′-(5-(trans-4-carboxy-4-methylcyclohexyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments, the inhibitor of PD-1/PD-L1 is a humanized antibody.

In some embodiments, the inhibitor of PD-1/PD-L1 is pembrolizumab.

In some embodiments, the inhibitor of PD-1/PD-L1 is nivolumab.

In some embodiments, the inhibitor of PD-1/PD-L1 is atezolizumab.

In some embodiments, the inhibitor of PD-1/PD-L1 is an antibody or antigen-binding fragment thereof that binds to human PD-1. In some embodiments, the antibody or antigen-binding fragment thereof that binds to human PD-1 is a humanized antibody.

In some embodiments, the inhibitor of PD-1/PD-L1 is retifanlimab (i.e., MGA-012).

Retifanlimab is a humanized IgG4 monoclonal antibody that binds to human PD-1. See hPD-1 mAb 7(1.2) in U.S. Pat. No. 10,577,422, which is incorporated herein by reference in its entirety. The amino acid sequences of the mature retifanlimab heavy and light chains are shown below. Complementarity-determining regions (CDRs) 1, 2, and 3 of the variable heavy (VH) domain and the variable light (VL) domain are shown in that order from N to the C-terminus of the mature VL and VH sequences and are both underlined and bolded. An antibody consisting of the mature heavy chain (SEQ ID NO:2) and the mature light chain (SEQ ID NO:3) listed below is termed retifanlimab.

Mature retifanlimab heavy chain (HC)
(SEQ ID NO: 2)
QVQLVQSGAEVKKPGASVKVSCKASGYSFT [symbol] WVRQAPG
QGLEWIG [symbol] [symbol] RVTITVDKSTSTAYMELS
SLRSEDTAVYYCAR [symbol] [symbol] WGQGTLVTVSSASTKGPS
VFPLAPCSRSTSESTAALGCLVKDYFPEPVTVSWNSGALTSG
VHTFPAVLQSSGLYSLSSVVTVPSSSLGTKTYTCNVDHKPSN
TKVDKRVESKYGPPCPPCPAPEFLGGPSVFLFPPKPKDTLMI
SRTPEVTCVVVDVSQEDPEVQFNWYVDGVEVHNAKTKPREEQ
FNSTYRVVSVLTVLHQDWLNGKEYKCKVSNKGLPSSIEKTIS
KAKGQPREPQVYTLPPSQEEMTKNQVSLTCLVKGFYPSDIAV
EWESNGQPENNYKTTPPVLDSDGSFFLYSRLTVDKSRWQEGN
VFSCSVMHEALHNHYTQKSLSLSLG
Mature retifanlimab light chain (LC)
(SEQ ID NO: 3)
EIVLTQSPATLSLSPGERATLSC [symbol] FQQ
KPGQPPKLLIH [symbol] [symbol] GVPSRFSGSGSGTDFTLTISSLEP
EDFAVYFC [symbol] FGGGTKVEIKRTVAAPSVFIFPPSD
EQLKSGTASVVCLLNNFYPREAKVQWKVDNALQSGNSQESVT
EQDSKDSTYSLSSTLTLSKADYEKHKVYACEVTHQGLSSPVT
KSFNRGEC

The variable heavy (VH) domain of retifanlimab has the following amino acid sequence:

(SEQ ID NO: 4)
QVQLVQSGAEVKKPGASVKVSCKASGYSFT [symbol] WVRQAPG
QGLEWIG [symbol] [symbol] RVTITVDKSTSTAYMELS
SLRSEDTAVYYCAR [symbol] [symbol] WGQGTLVTVSS

The variable light (VL) domain of retifanlimab has the following amino acid sequence:

(SEQ ID NO: 5)
EIVLTQSPATLSLSPGERATLSC [symbol] FQQ
KPGQPPKLLIH [symbol] [symbol] GVPSRFSGSGSGTDFTLTISSLEP
EDFAVYFC [symbol] FGGGTKVEIK

The amino acid sequences of the VH CDRs of retifanlimab are listed below:

VH CDR1:
(SEQ ID NO: 6)
SYWMN;
VH CDR2:
(SEQ ID NO: 7)
VIHPSDSETWLDQKFKD;
VH CDR3:
(SEQ ID NO: 8)
EHYGTSPFAY

The amino acid sequences of VL CDRs of retifanlimab are listed below:

VL CDR1:
(SEQ ID NO: 9)
RASESVDNYGMSFMNW;
VL CDR2:
(SEQ ID NO: 10)
AASNQGS;
and
VL CDR3:
(SEQ ID NO: 11)
QQSKEVPYT.

In some embodiments, the inhibitor of PD-1/PD-L1 is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:

the VH CDR1 comprises the amino acid sequence SYWMN (SEQ ID NO:6);

the VH CDR2 comprises the amino acid sequence VIHPSDSETWLDQKFKD (SEQ ID NO:7); and

the VH CDR3 comprises the amino acid sequence EHYGTSPFAY (SEQ ID NO:8); and

wherein the antibody comprises a variable light (VL) domain comprising VL CDR1, VL CDR2, and VL CDR3, wherein:

the VL CDR1 comprises the amino acid sequence RASESVDNYGMSFMNW (SEQ ID NO:9);

the VL CDR2 comprises the amino acid sequence AASNQGS (SEQ ID NO:10); and

the VL CDR3 comprises the amino acid sequence QQSKEVPYT (SEQ ID NO:11).

### Anti-CD73 Antibodies

CD73 (also known as “5′-nucleotidase” and “ecto-5′-nucleotidase”) is a dimeric enzyme (EC:3.1.3.5) that functions as a homodimer bound by a GPI linkage to the external face of the plasma membrane. CD73 can be shed and is active as a soluble protein in circulation. CD73 catalyzes the conversion of extracellular AMP to adenosine. CD73 enzymatic activity requires substrate binding in the open CD73 conformation. After the substrate binding, CD73 goes through a large conformational change from open to closed conformation to convert AMP to adenosine (see, e.g., Knapp et al., 2012, Structure, 20(12):2161-73). CD73 also functions as a cellular adhesion molecule and plays a role in regulation of leukocyte trafficking.

CD73 enzymatic activity plays a role in the promotion and metastasis of cancer (see, e.g., Stagg and Smyth, 2010, Oncogene, 29:5346-5358; Salmi and Jalkanen, 2012, Oncolmmunology, 1:247-248, 2012; Stagg, 2012, Oncolmmunology, 1:217-218; Zhang, 2012, Oncolmmunology, 167-70). Overexpression of CD73 in cancer cells impairs adaptive antitumor immune responses, enhancing tumor growth and metastasis (see, e.g., Niemela et al., 2004, J Immunol., 172:1646-1653; Sadej et al., 2006, Nucleosides Nucleotides Nucleic Acids, 25:1119-1123; Braganhol et al., 2007, Biochim. Biophys. Acta., 1770:1352-1359; Zhang, 2010, Cancer Res., 70:6407-6411; Zhang, 2012, Oncolmmunology, 1:67-70).

An exemplary amino acid sequence of the mature human CD73 protein (amino acids 27-549 of GenBank Accession No. NP_002517) is:

(SEQ ID NO: 70)
WELTILHTNDVHSRLEQTSEDSSKCVNASRCMGGV
ARLFTKVQQIRRAEPNVLLLDAGDQYQGTIWFTVY
KGAEVAHFMNALRYDAMALGNHEFDNGVEGLIEPL
LKEAKFPILSANIKAKGPLASQISGLYLPYKVLPV
GDEVVGIVGYTSKETPFLSNPGTNLVFEDEITALQ
PEVDKLKTLNVNKIIALGHSGFEMDKLIAQKVRGV
DVVVGGHSNTFLYTGNPPSKEVPAGKYPFIVTSDD
GRKVPVVQAYAFGKYLGYLKIEFDERGNVISSHGN
PILLNSSIPEDPSIKADINKWRIKLDNYSTQELGK
TIVYLDGSSQSCRFRECNMGNLICDAMINNNLRHT
DEMFWNHVSMCILNGGGIRSPIDERNNGTITWENL
AAVLPFGGTFDLVQLKGSTLKKAFEHSVHRYGQST
GEFLQVGGIHVVYDLSRKPGDRVVKLDVLCTKCRV
PSYDPLKMDEVYKVILPNFLANGGDGFQMIKDELL
RHDSGDQDINVVSTYISKMKVIYPAVEGRIKFS.

An exemplary amino acid sequence of the mature murine CD73 protein (amino acids 29-551 of GenBank Accession No. NP_035981) is:

(SEQ ID NO: 71)
WELTILHTNDVHSRLEQTSDDSTKCLNASLCVGGV
ARLFTKVQQIRKEEPNVLFLDAGDQYQGTIWFTVY
KGLEVAHFMNILGYDAMALGNHEFDNGVEGLIDPL
LRNVKFPILSANIKARGPLAHQISGLFLPSKVLSV
GGEVVGIVGYTSKETPFLSNPGTNLVFEDEISALQ
PEVDKLKTLNVNKIIALGHSGFEMDKLIAQKVRGV
DIVVGGHSNTFLYTGNPPSKEVPAGKYPFIVTADD
GRQVPVVQAYAFGKYLGYLKVEFDDKGNVITSYGN
PILLNSSIPEDATIKADINQWRIKLDNYSTQELGR
TIVYLDGSTQTCRFRECNMGNLICDAMINNNLRHP
DEMFWNHVSMCIVNGGGIRSPIDEKNNGTITWENL
AAVLPFGGTFDLVQLKGSTLKKAFEHSVHRYGQST
GEFLQVGGIHVVYDINRKPWNRVVQLEVLCTKCRV
PIYEPLEMDKVYKVTLPSYLANGGDGFQMIKDELL
KHDSGDQDISVVSEYISKMKVVYPAVEGRIKFS.

An exemplary amino acid sequence of the mature cynomolgus CD73 protein is:

(SEQ ID NO: 72)
WELTILHTNDVHSRLEQTSEDSSKCVNASRCMGGV
ARLFTKVQQIRRAEPNVLLLDAGDQYQGTIWFTVY
KGAEVAHFMNALRYDAMALGNHEFDNGVEGLIEPL
LKEAKFPILSANIKAKGPLASQISGLYLPYKVLPV
GDEVVGIVGYTSKETPFLSNPGTNLVFEDEITALQ
PEVDKLKTLNVNKIIALGHSGFETDKLIAQKVRGV
DVVVGGHSNTFLYTGNPPSKEVPAGKYPFIVTSDD
GRKVPVVQAYAFGKYLGYLKIEFDERGNVISSHGN
PILLNSSIPEDPSIKADINKWRIKLDNYSTQELGK
TIVYLDGSSQSCRFRECNMGNLICDAMINNNLRHA
DEMFWNHVSMCILNGGGIRSPIDERNNGTITWENL
AAVLPFGGTFDLVQLKGSTLKKAFEHSVHRYGQST
GEFLQVGGIHVVYDLSRKPGDRVVKLDVLCTKCRV
PSYDPLKMDEIYKVILPNFLANGGDGFQMIKDELL
RHDSGDQDINVVSTYISKMKVIYPAVEGRIKFS.

This disclosure provides anti-CD73 antibodies that are useful in combination with an A2A and/or A2B adenosine receptor inhibitor and a PD-1/PD-L1 inhibitor in treating diseases, e.g., cancer. This disclosure further provides anti-CD73 antibodies that are useful in combination with a PD-1/PD-L1 inhibitor in treating diseases, e.g., cancer. These anti-CD73 antibodies can bind human CD73.

In some instances, these antibodies bind human CD73 and cynomolgus CD73. In some instances, these antibodies bind human CD73 and cynomolgus CD73 and do not bind murine CD73. Such anti-CD73 antibodies include the sequences of an anti-CD73 monoclonal antibody, CL25, and a humanized version thereof, HzCL25 (i.e., ANTIBODY Y), which humanized version thereof binds with high affinity to both human and cynomolgus CD73, and has undetectable binding to mouse CD73.

In some instances, these antibodies bind human CD73, cynomolgus CD73, and murine CD73. Such anti-CD73 antibodies includes the sequences of a human anti-CD73 monoclonal antibody, 3-F03, which binds with high affinity to the open conformation of each of human, cynomolgus, and murine CD73.

### Antibody HzCL25 (i.e., ANTIBODY Y)

Antibody HzCL25 is a humanized IgG1/kappa monoclonal antibody with alanine at position Asparagine-297 (N297, according to EU numbering) of the heavy chain constant region to reduce effector function. It specifically binds human and cynomolgus CD73 with high affinity (K_{D} 0.5 nM) and has low effector functionality.

HzCL25 was constructed from a chimeric version of the murine CL25 antibody.

Table 2, below, shows the amino acid sequences of the HzCL25 CDRs according to IMGT numbering. Table 2, below, also shows the amino acid sequences of the HzCL25 mature VH, VL, heavy chain, and light chain.

TABLE 2
Amino acid sequences of HzCL25 CDRs,
VH, VL, heavy chain, and light chain

| | IMGT |
|---|---|
| VH | GYTFTSYG |
| CDR1 | (SEQ ID NO: 16) |
| VH | IYPGS GNT |
| CDR2 | (SEQ ID NO: 17) |
| VH | ARYDYL GS SYGFDY |
| CDR3 | (SEQ ID NO: 18) |
| VL | QDVSTA |
| CDR1 | (SEQ ID NO: 19) |
| VL | SAS |
| CDR2 | (SEQ ID NO: 20) |
| VL | QQHYNTPYT |
| CDR3 | (SEQ ID NO: 21) |
| VH | EVQLVQSGAEVKKPGESLKI |
| | SCKGSGYTFTSYGLSWVRQM |
| | PGKGLEWMGEIYPGSGNTYY |
| | NEKFKGQVTISADKSISTAY |
| | LQWSSLKASDTAMYYCARYD |
| | YLGSSYGFDYWGAGTTVTVS |
| | S |
| | (SEQ ID NO: 22) |
| VL | DIVMTQSPDSLAVSLGERAT |
| | INCKASQDVSTAVAWYQQKP |
| | GQPPKLLIYSASYRYSGVPD |
| | RFSGSGSGTDFTLTISSLQA |
| | EDVAVYYCQQHYNTPYTFGG |
| | GTKLEIK |
| | (SEQ ID NO: 23) |
| Heavy | EVQLVQSGAEVKKPGESLKI |
| Chain | SCKGSGYTFTSYGLSWVRQM |
| | PGKGLEWMGEIYPGSGNTYY |
| | NEKFKGQVTISADKSISTAYL |
| | QWSSLKASDTAMYYCARYDY |
| | LGSSYGFDYWGAGTTVTVSS |
| | ASTKGPSVFPLAPSSKSTSGG |
| | TAALGCLVKDYFPEPVTVSW |
| | NSGALTSGVHTFPAVLQSSG |
| | LYSLSSVVTVPSSSLGTQTY |
| | ICNVNHKPSNTKVDKRVEPK |
| | SCDKTHTCPPCPAPELLGGP |
| | SVFLFPPKPKDTLMISRTPE |
| | VTCVVVDVSHEDPEVKFNWY |
| | VDGVEVHNAKTKPREEQYAS |
| | TYRVVSVLTVLHQDWLNGKE |
| | YKCKVSNKALPAPIEKTISK |
| | AKGQPREPQVYTLPPSREEM |
| | TKNQVSLTCLVKGFYPSDIA |
| | VEWESNGQPENNYKTTPPVL |
| | DSDGSFFLYSKLTVDKSRWQ |
| | QGNVFSCSVMHEALHNHYTQ |
| | KSLSLSPG |
| | (SEQ ID NO: 24) |
| Light | DIVMTQSPDSLAVSLGERAT |
| Chain | INCKASQDVSTAVAWYQQKP |
| | GQPPKLLIYSASYRYSGVPD |
| | RFS |
| | GSGSGTDFTLTISSLQAEDV |
| | AVYYCQQHYNTPYTFGGGTK |
| | LEIKRTVAAPSVFIFPPSDE |
| | QLKSGTASVVCLLNNFYPRE |
| | AKVQWKVDNALQSGNSQESV |
| | TEQDSKDSTYSLSSTLTLSK |
| | ADYEKHKVYACEVTHQGLSS |
| | PVTKSFNRGEC |
| | (SEQ ID NO: 25) |

The anti-CD73 antibodies can encompass the VH CDR1, VH CDR2, and VH CDR3 and the VL CDR 1, VL CDR2, and VL CDR3 of HzCL25. In some instances, the anti-CD73 antibody comprises a VH comprising VH CDR1, VH CDR2, and VH CDR3 of HzCL25 (see Table 2). In some instances, the anti-CD73 antibody comprises a VL comprising VL CDR1, VL CDR2, and VL CDR3 of HzCL25 (see Table 2). In some instances, the anti-CD73 antibody comprises a VH comprising VH CDR1, VH CDR2, and VH CDR3 of HzCL25 (see Table 2) and a VL comprising VL CDR1, VL CDR2, and VL CDR3 of HzCL25 (see Table 2). In some instances, the anti-CD73 antibodies can have, e.g., 1, 2, or 3 substitutions within one or more (i.e., 1, 2, 3, 4, 5, or 6) of the six CDRs of HzCL25. In some instances, the antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control); and/or (iii) bind human or cynomolgus monkey CD73 in the open conformation with high affinity (e.g., K_{D}≤0.5 nM) but do not significantly bind CD73 in the open conformation from mice; and/or (iv) bind human or cynomolgus monkey CD73 in the closed conformation with high affinity (e.g., K_{D}≤0.5 nM) but do not significantly bind CD73 in the closed conformation from mice; and/or (v) bind to an epitope within amino acids 40-53 of SEQ ID NO:70 (i.e., within TKVQQIRRAEPNVL (SEQ ID NO:76)); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control); and/or (ix) reduce free surface CD73 on cells (e.g., cancer cells, e.g., melanoma cancer cancers, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control).

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22). In certain embodiments, the anti-CD73 antibodies comprise a VH comprising the VH CDR1, VH CDR2, and VH CDR3 of HzCL25 (i.e., the amino acid sequences set forth in SEQ ID NOs: 16-18, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22). In certain embodiments, the anti-CD73 antibodies comprise a VH comprising the amino acid sequence set forth in SEQ ID NO:22. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24). In some embodiments, the anti-CD73 antibodies comprise a heavy chain comprising a VH comprising the VH CDR1, VH CDR2, and VH CDR3 of HzCL25 (i.e., the amino acid sequences set forth in SEQ ID NOs: 16-18, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24). In certain embodiments, the anti-CD73 antibodies comprise a heavy chain comprising the amino acid sequence set forth in SEQ ID NO:24. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise a VL comprising the VL CDR1, VL CDR2, and VL CDR3 of HzCL25 (i.e., the amino acid sequences set forth in SEQ ID NOs: 19-21, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise a VL comprising the amino acid sequence set forth in SEQ ID NO:23. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In some embodiments, the anti-CD73 antibodies comprise a light chain comprising a VL comprising the VL CDR1, VL CDR2, and VL CDR3 of HzCL25 (i.e., the amino acid sequences set forth in SEQ ID NOs: 19-21, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In certain embodiments, the anti-CD73 antibodies comprise a light chain comprising the amino acid sequence set forth in SEQ ID NO:25. In certain embodiments, the anti-CD73 antibodies comprise: (i) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22); and (ii) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise: (i) a VH comprising the VH CDR1, VH CDR2, and VH CDR3 of HzCL25 (i.e., the amino acid sequences set forth in SEQ ID NOs: 16-18, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22), and (ii) a VL comprising the VL CDR1, VL CDR2, and VL CDR3 of HzCL25 (i.e., the amino acid sequences set forth in SEQ ID NOs: 19-21, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise: a VH comprising the amino acid sequence set forth in SEQ ID NO:22, and (ii) a VL comprising the amino acid sequence set forth in SEQ ID NO:23. In some embodiments, the anti-CD73 antibodies comprise: (i) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24); and (ii) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In some embodiments, the anti-CD73 antibodies comprise: (i) a heavy chain comprising a VH comprising the VH CDR1, VH CDR2, and VH CDR3 of HzCL25 (i.e., the amino acid sequences set forth in SEQ ID NOs: 16-18, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24), and (ii) a light chain comprising a VL comprising the VL CDR1, VL CDR2, and VL CDR3 of HzCL25 (i.e., the amino acid sequences set forth in SEQ ID NOs: 19-21, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In some embodiments, the anti-CD73 antibodies comprise: (i) a heavy chain comprising the amino acid sequence set forth in SEQ ID NO:24, and (ii) a light chain comprising the amino acid sequence set forth in SEQ ID NO:25.

The CD73-binding epitope of HzCL25 is within the amino acid sequence TKVQQIRRAEPNVL (SEQ ID NO:76) (i.e., amino acids 40-53 of the amino acid sequence set forth in SEQ ID NO:70). This disclosure features antibodies that bind to CD73 within the sequence TKVQQIRRAEPNVL (SEQ ID NO:76). This disclosure features antibodies that bind to the same epitope as HzCL25. This disclosure also features antibodies that competitively inhibit binding of HzCL25 to human CD73. In some embodiments, the VH of HzCL25 is linked to a heavy chain constant region comprising a CH1 domain and a hinge region. In some embodiments, the VH of HzCL25 is linked to a heavy chain constant region comprising a CH3 domain. In some embodiments, the CH3 domain lacks the C-terminal lysine (K) amino acid residue. In some embodiments, the CH3 domain contains the C-terminal lysine (K) amino acid residue. In certain embodiments, the VH of HzCL25 is linked to a heavy chain constant region comprising a CH1 domain, hinge region, CH2 domain, and CH3 domain from human IgG1. In some embodiments, the CH₃ domain from human IgG1 lacks the C-terminal lysine (K) amino acid residue. In some embodiments, the CH₃ domain from human IgG1 contains the C-terminal lysine (K) amino acid residue. In certain embodiments such an antibody contains one or more additional mutations in the heavy chain constant region that increase the stability of the antibody. In certain embodiments, the heavy chain constant region includes substitutions that modify the properties of the antibody (e.g., decrease Fc receptor binding, increase or decrease antibody glycosylation, decrease binding to C1q). In certain embodiments, the heavy chain constant region includes an alanine at position Asparagine-297 (N297, according to EU numbering) of the heavy chain constant region to reduce effector function.

In certain embodiments, the anti-CD73 antibody is an IgG antibody. In one embodiment, the antibody is an IgG1 antibody. In one embodiment, the antibody is an IgG4 antibody. In another embodiment, the antibody is an IgG2 antibody. In certain embodiments, the anti-CD73 antibody comprises a heavy chain constant region lacking one or more lysine (K) amino acid residues relative to a wild type heavy chain constant region. For example, in certain embodiments, the antibody comprises heavy chain constant region lacking the C-terminal lysine (K) amino acid residue of the CH₃ domain of the heavy chain constant region.

### Antibody 3-F03

Antibody 3-F03 is a human IgG1/kappa monoclonal antibody with alanine at position Asparagine-297 (N297, according to EU numbering) of the heavy chain constant region to reduce effector function. 3-F03 specifically binds human, cynomolgus, and murine CD73 with high affinity (K_{D}≤2 nM) and has low effector functionality.

Table 3, below, shows the amino acid sequences of the 3-F03 CDRs according to IMGT numbering. Table 3, below, also shows the amino acid sequences of the 3-F03 mature VH, VL, heavy chain, and light chain.

TABLE 3
Amino acid sequences of 3-F03 CDRs,
VH, and VL

| | IMGT |
|---|---|
| VH CDR1 | GFTFSSYD |
| | (SEQ ID NO: 34) |
| VH CDR2 | MSYDGSNK |
| | (SEQ ID NO: 35) |
| VH CDR3 | ATEIAAKGDY |
| | (SEQ ID NO: 36) |
| VL CDR1 | QGISNY |
| | (SEQ ID NO: 37) |
| VL CDR2 | AAS |
| | (SEQ ID NO: 38) |
| VL CDR3 | QQSYSTPH |
| | (SEQ ID NO: 39) |
| VH | VQLVESGGGLVQPGGSLRLSCAASG |
| | FTFSSYDMHWVRQAPGKGLEWVAVM |
| | SYDGSNKYYADSVKGRFTISRDNSK |
| | NALYLQMNSLRAEDTAVYYCATEIA |
| | AKGDYWGQGTLVTVSS |
| | (SEQ ID NO: 60) |
| VL | IQMTQSPSSLSASVGDRVTITCRAS |
| | QGISNYLAWYQQKPGKAPKLLIYAA |
| | STLQSGVPSRFSGSGSGTDFTLTIS |
| | SLQPEDFATYYCQQSYSTPHFGQGT |
| | RLEIK |
| | (SEQ ID NO: 61) |
| HC | VQLVESGGGLVQPGGSLRLSCAASG |
| | FTFSSYDMHWVRQAPGKGLEWVAVM |
| | SYDGSNKYYADSVKGRFTISRDNSK |
| | NALYLQMNSLRAEDTAVYYCATEIA |
| | AKGDYWGQGTLVTVSSASTKGPSVF |
| | PLAPSSKSTSGGTAALGCLVKDYFP |
| | EPVTVSWNSGALTSGVHTFPAVLQS |
| | SGLYSLSSVVTVPSSSLGTQTYICN |
| | VNHKPSNTKVDKRVEPKSCDKTHTC |
| | PPCPAPELLGGPSVFLFPPKPKDTL |
| | MISRTPEVTCVVVDVSHEDPEVKFN |
| | WYVDGVEVHNAKTKPREEQYASTYR |
| | VVSVLTVLHQDWLNGKEYKCKVSNK |
| | ALPAPIEKTISKAKGQPREPQVYTL |
| | PPSREEMTKNQVSLTCLVKGFYPSD |
| | IAVEWESNGQPENNYKTTPPVLDSD |
| | GSFFLYSKLTVDKSRWQQGNVFSCS |
| | VMHEALHNHYTQKSLSLSPGK |
| | (SEQ ID NO: 66) |
| LC | IQMTQSPSSLSASVGDRVTITCRAS |
| | QGISNYLAWYQQKPGKAPKLLIYAA |
| | STLQSGVPSRFSGSGSGTDFTLTIS |
| | SLQPEDFATYYCQQSYSTPHFGQGT |
| | RLEIKRTVAAPSVFIFPPSDEQLKS |
| | GTASVVCLLNNFYPREAKVQWKVDNA |
| | LQSGNSQESVTEQDSKDSTYSLSST |
| | LTLSKADYEKHKVYACEVTHQGLSS |
| | PVTKSFNRGEC |
| | (SEQ ID NO: 31) |

Variants of 3-F03 are also described herein. 3-F03_411 is identical to 3-F03, except that the 3-F03_411 heavy chain (i) contains an N-terminal glutamate (E) that is lacking in 3-F03 and (ii) does not include the C-terminal lysine present in 3-F03. Table 4, below, shows the amino acid sequences of the 3-F03_411 mature VH, VL, heavy chain and light chain. 3-F03_413 is identical to 3-F03_411, except that it contains a glutamate (E) at VH Kabat position H53 (position 54 of SEQ TD NO:60) instead of an aspartic acid (D). Table 5, below, shows the amino acid sequences of the 3-F03_413 CDRs according to IMGT, Chothia, AbM, Kabat, and Contact numbering. Table 5, below, also shows the amino acid sequences of the 3-F03_413 mature VH, VL, heavy chain, and light chain.

TABLE 4
Amino acid sequences of 3-F03_411 HC and LC

| | SEQUENCE |
|---|---|
| VH | EVQLVESGGGLVQPGGSLRL |
| | SCAASGFTFSSYDMHWVRQA |
| | PGKGLEWVAVMSYDGSNKYY |
| | ADSVKGRFTISRDNSKNALY |
| | LQMNSLRAEDTAVYYCATEI |
| | AAKGDYWGQGTLVTVSS |
| | (SEQ ID NO: 62) |
| VL | IQMTQSPSSLSASVGDRVTI |
| | TCRASQGLSNYLAWYQQKPG |
| | KAPKLLIYAASTLQSGVPSR |
| | FSGSGSGTDFTLTISSLQPE |
| | DFATYYCQQSYSTPHFGQGT |
| | RLEIK |
| | (SEQ ID NO: 61) |
| Heavy Chain | EVQLVESGGGLVQPGGSLRL |
| | SCAASGFTFSSYDMHWVRQA |
| | PGKGLEWVAVMSYDGSNKYY |
| | ADSVKGRFTISRDNSKNALY |
| | LQMNSLRAEDTAVYYCATEI |
| | AAKGDYWGQGTLVTVSSAST |
| | KGPSVFPLAPSSKSTSG |
| | GTAALGCLVKDYFPEPVTVS |
| | WNSGALTSGVHTFPAVLQSS |
| | GLYSLSSVVTVPSSSLGTQT |
| | YICNVNHKPSNTKVDKRVEP |
| | KSCDKTHTCPPCPAPELLGG |
| | PSVFLFPPKPKDTLMISRTP |
| | EVTCVVVDVSHEDPEVKFNW |
| | YVDGVEVHNAKTKPREEQYA |
| | STYRVVSVLTVLHQDWLNGK |
| | EYKCKVSNKALPAPIEKTIS |
| | KAKGQPREPQVYTLPPSREE |
| | MTKNQVSLTCLVKGFYPSDI |
| | AVEWESNGQPENNYKTTPPV |
| | LDSDGSFFLYSKLTVDKSRW |
| | QQGNVFSCSVMHEALHNHYT |
| | QKSLSLSPG |
| | (SEQ ID NO: 30) |
| Light Chain | IQMTQSPSSLSASVGDRVTI |
| | TCRASQGISNYLAWYQQKPG |
| | KAPKLLIYAASTLQSGVPSR |
| | FSGSGSGTDFTLTISSLQPE |
| | DFATYYCQQSYSTPHFGQGT |
| | RLEIKRTVAAPSVFIFPPSD |
| | EQLKSGTASVVCLLNNFYPR |
| | EAKVQWKVDNALQSGNSQES |
| | VTEQDSKDSTYSLSSTLTLS |
| | KADYEKHKVYACEVTHQGLS |
| | SPVTKSFNRGEC |
| | (SEQ ID NO: 31) |

TABLE 5
Amino acid sequences of 3-F03_413
CDRs, VH, VL, HC, LC

| | IMGT |
|---|---|
| VH CDR1 | GFTFSSYD |
| | (SEQ ID NO: 34) |
| VH CDR2 | MSYEGSNK |
| | (SEQ ID NO:40) |
| VH CDR3 | ATEIAAKGDY |
| | (SEQ ID NO:36) |
| VL CDR1 | QGISNY |
| | (SEQ ID NO:37) |
| VL CDR2 | AAS |
| | (SEQ ID NO:38) |
| VL CDR3 | QQSYSTPH |
| | (SEQ ID NO:39) |
| VH | EVQLVESGGGLVQPGGSLRL |
| | SCAASGFTFSSYDMHWVRQA |
| | PGKGLEWVAVMSYEGSNKYY |
| | ADSVKGRFTISRDNSKNALY |
| | LQMNSLRAEDTAVYYCATEI |
| | AAKGDYWGQGTLVTVSS |
| | (SEQ ID NO: 63) |
| VL | IQMTQSPSSLSASVGDRVTI |
| | TCRASQGISNYLAWYQQKPG |
| | KAPKLLIYAASTLQSGVPSR |
| | FSGSGSGTDFTLTISSLQPE |
| | DFATYYCQQSYSTPHFGQGT |
| | RLEIK |
| | (SEQ ID NO: 61) |
| HC | EVQLVESGGGLVQPGGSLRL |
| | SCAASGFTFSSYDMHWVRQA |
| | PGKGLEWVAVMSYEGSNKYY |
| | ADSVKGRFTISRDNSKNALY |
| | LQMNSLRAEDTAVYYCATEI |
| | AAKGDYWGQGTLVTVSSAST |
| | KGPSVFPLAPSSKSTSGGTA |
| | ALGCLVKDYFPEPVTVSWNS |
| | GALTSGVHTFPAVLQSSGLY |
| | SLSSVVTVPSSSLGTQTYIC |
| | NVNHKPSNTKVDKRVEPKSC |
| | DKTHTCPPCPAPELLGGPSV |
| | FLFPPKPKDTLMISRTPEVT |
| | CVVVDVSHEDPEVKFNWYVD |
| | GVEVHNAKTICPREEQYAST |
| | YRVVSVLTVLHQDWLNGKEY |
| | KCKVSNKALPAPIEKTISKA |
| | KGQPREPQVYTLPPSREEMT |
| | KNQVSLTCLVKGFYPSDIAV |
| | EWESNGQPENNYKTTPPVLD |
| | SDGSFFLYSKLTVDKSRWQQ |
| | GNVFSCSVMHEALHNHYTQK |
| | SLSLSPG |
| | (SEQ ID NO: 33) |
| LC | IQMTQSPSSLSASVGDRVTI |
| | TCRASQGISNYLAWYQQKPG |
| | KAPKLLIYAASTLQSGVPSR |
| | FSGSGSGTDFTLTISSLQPE |
| | DFATYYCQQSYSTPHFGQGT |
| | RLEIKRTVAAPSVFIFPPSD |
| | EQLKSGTASVVCLLNNFYPR |
| | EAKVQWKVDNALQSGNSQES |
| | VTEQDSKDSTYSLSSTLTLS |
| | KADYEKHKVYACEVTHQGLS |
| | SPVTKSFNRGEC |
| | (SEQ ID NO: 31) |

The anti-CD73 antibodies can encompass the VH CDR1, VH CDR2, and VH CDR3 and the VL CDR1, VL CDR2, and VL CDR3 of 3-F03 or 3-F03_413. In some instances, the anti-CD73 antibody comprises a VH comprising VH CDR1, VH CDR2, and VH CDR3 of 3-F03 (see Table 3). In some instances, the anti-CD73 antibody comprises a VL comprising VL CDR1, VL CDR2, and VL CDR3 of 3-F03 (see Table 3).

In some instances, the anti-CD73 antibody comprises a VH comprising VH CDR1, VH CDR2, and VH CDR3 of 3-F03 (see Table 3) and a VL comprising VL CDR1, VL CDR2, and VL CDR3 of 3-F03 (see Table 3). In some instances, the anti-CD73 antibody comprises a VH comprising VH CDR1, VH CDR2, and VH CDR3 of 3-F03_413 (see Table 5). In some instances, the anti-CD73 antibody comprises a VL comprising VL CDR1, VL CDR2, and VL CDR3 of 3-F03_413 (see Table 5). In some instances, the anti-CD73 antibody comprises a VH comprising VH CDR1, VH CDR2, and VH CDR3 of 3-F03_413 (see Table 5) and a VL comprising VL CDR1, VL CDR2, and VL CDR3 of 3-F03_413 (see Table 5). In some instances, the anti-CD73 antibodies can have, e.g., 1, 2, or 3 substitutions within one or more (i.e., 1, 2, 3, 4, 5, or 6) of the six CDRs of 3-F03 or 3-F03_413. In some instances, these antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control); and/or (iii) bind human, cynomolgus monkey, or murine CD73 in the open conformation with high affinity (e.g., K_{D}≤2 nM); and/or (iv) do not bind human, cynomolgus monkey, or murine CD73 in the closed conformation; and/or (v) bind to an epitope within amino acids 386-399 of SEQ ID NO:70 (i.e., within AAVLPFGGTFDLVQ (SEQ ID NO:78) amino acids 470-489 of SEQ ID NO:70 (i.e., within ILPNFLANGGDGFQMIKDEL (SEQ ID NO:79)); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control).

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 or 3-F03_413 (i.e., the amino acid sequence set forth in SEQ ID NO:62 or 63, respectively). In certain embodiments, the anti-CD73 antibodies comprise a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 (i.e., the amino acid sequence set forth in SEQ ID NO:62). In certain embodiments, the anti-CD73 antibodies comprise a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 (i.e., the amino acid sequence set forth in SEQ ID NO:63). In some embodiments, the anti-CD73 antibodies comprise a VH comprising the amino acid sequence set forth in SEQ ID NO:62. In some embodiments, the anti-CD73 antibodies comprise a VH comprising the amino acid sequence set forth in SEQ ID NO:63. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 or 3-F03_F13 (i.e., the amino acid sequence set forth in SEQ ID NO:30 or 33, respectively). In some embodiments, the anti-CD73 antibodies comprise a heavy chain comprising a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 (i.e., the amino acid sequence set forth in SEQ ID NO:30). In some embodiments, the anti-CD73 antibodies comprise a heavy chain comprising a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_413 (i.e., the amino acid sequence set forth in SEQ ID NO:33). In some embodiments, the anti-CD73 antibodies comprise a heavy chain comprising the amino acid sequence set forth in SEQ ID NO:30. In some embodiments, the anti-CD73 antibodies comprise a heavy chain comprising the amino acid sequence set forth in SEQ ID NO:33. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_411 or 3-F03_413 (i.e., the amino acid sequence set forth in SEQ ID NO:61). In certain embodiments, the anti-CD73 antibodies comprise a VL comprising the VL CDR1, VL CDR2, and VL CDR3 of 3-F03_411 or 3-F03_413 (see Table 3, e.g., according to the IMGT definition, i.e., the amino acid sequences set forth in SEQ ID NOs: 37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_411 or 3-F03_413 (i.e., the amino acid sequence set forth in SEQ ID NO:61). In some embodiments, the anti-CD73 antibodies comprise a VL comprising the amino acid sequence set forth in SEQ ID NO:61. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_411 or 3-F03_413 (i.e., the amino acid sequence set forth in SEQ ID NO:31). In some embodiments, the anti-CD73 antibodies comprise a light chain comprising a VL comprising the VL CDR1, VL CDR2, and VL CDR3 of 3-F03_411 or 3-F03_413 (see Table 5, e.g., according to the IMGT definition, i.e., the amino acid sequences set forth in SEQ ID NOs: 37-39, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_411 or 3-F03_413 (i.e., the amino acid sequence set forth in SEQ ID NO:31). In some embodiments, the anti-CD73 antibodies comprise a light chain comprising the amino acid sequence set forth in SEQ ID NO:31.

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 or 3-F03_413 (i.e., the amino acid sequence set forth in SEQ ID NO:62 or 63, respectively) and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_411 or 3-F03_413 (i.e., the amino acid sequence set forth in SEQ ID NO:61). In certain embodiments, the anti-CD73 antibodies comprise: (i) a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03 (i.e., the amino acid sequence set forth in SEQ ID NO:62), and (ii) a VL comprising the VL CDR1, VL CDR2, and VL CDR3 of 3-F03_411 (see Table 3, e.g., according to the IMGT definition, i.e., the amino acid sequences set forth in SEQ ID NOs: 37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03 (i.e., the amino acid sequence set forth in SEQ ID NO:61). In certain embodiments, the anti-CD73 antibodies comprise: (i) a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_413 (i.e., the amino acid sequence set forth in SEQ ID NO:63), and (ii) a VL comprising the VL CDR1, VL CDR2, and VL CDR3 of 3-F03_413 (see Table 5, e.g., according to the IMGT definition, i.e., the amino acid sequences set forth in SEQ ID NOs: 37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_413 (i.e., the amino acid sequence set forth in SEQ ID NO:61). In some embodiments, the anti-CD73 antibody comprises: (i) a VH comprising the amino acid sequence set forth in SEQ ID NO:62; and (ii) a VL comprising the amino acid sequence set forth in SEQ ID NO:61. In some embodiments, the anti-CD73 antibody comprises: (i) a VH comprising the amino acid sequence set forth in SEQ ID NO:63; and (ii) a VL comprising the amino acid sequence set forth in SEQ ID NO:61. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 or 3-F03_413 (i.e., the amino acid sequence set forth in SEQ ID NO:30 or 33) and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_411 or 3-F03_413 (i.e., the amino acid sequence set forth in SEQ ID NO:31). In some embodiments, the anti-CD73 antibodies comprise: (i) a heavy chain comprising the a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 (i.e., the amino acid sequence set forth in SEQ ID NO:30), and (ii) a light chain comprising a VL comprising the VL CDR1, VL CDR2, and VL CDR3 of 3-F03_411 (see Table 3, e.g., according to the IMGT definition, i.e., the amino acid sequences set forth in SEQ ID NOs: 37-39, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03 (i.e., the amino acid sequence set forth in SEQ ID NO:31). In some embodiments, the anti-CD73 antibodies comprise: (i) a heavy chain comprising the a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03 (i.e., the amino acid sequence set forth in SEQ ID NO:33), and (ii) a light chain comprising a VL comprising the VL CDR1, VL CDR2, and VL CDR3 of 3-F03_413 (see Table 5, e.g., according to the IMGT definition, i.e., the amino acid sequences set forth in SEQ ID NOs: 37-39, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_413 (i.e., the amino acid sequence set forth in SEQ ID NO:31). In some embodiments, the anti-CD73 antibody comprises: (i) a heavy chain comprising the amino acid sequence set forth in SEQ ID NO:30; and (ii) a light chain comprising the amino acid sequence set forth in SEQ ID NO:31. In some embodiments, the anti-CD73 antibody comprises: (i) a heavy chain comprising the amino acid sequence set forth in SEQ ID NO:33; and (ii) a light chain comprising the amino acid sequence set forth in SEQ ID NO:31.

The CD73-binding epitope of 3-F03 (and variants thereof, e.g., 3-F03_411 and 3-F03_413) contains AAVLPFGGTFDLVQ (SEQ ID NO:78) (i.e., amino acids 386-399 of the amino acid sequence set forth in SEQ ID NO:70) and ILPNFLANGGDGFQMIKDEL (SEQ ID NO:79) (i.e., amino acids 470-489 of the amino acid sequence set forth in SEQ ID NO:70). This disclosure features antibodies that bind to CD73 an epitope within AAVLPFGGTFDLVQ (SEQ ID NO:78) and ILPNFLANGGDGFQMIKDEL (SEQ ID NO:79). This disclosure features antibodies that bind to the same epitope as 3-F03 (or a variant thereof, e.g., 3-F03_411 or 3-F03_413). This disclosure also features antibodies that competitively inhibit binding of 3-F03 (or a variant thereof, e.g., 3-F03_411 or 3-F03_413) to human CD73.

In some embodiments, the VH of 3-F03 (or a variant thereof, e.g., 3-F03_411 or 3-F03_413) is linked to a heavy chain constant region comprising a CH1 domain and a hinge region. In some embodiments, the VH of 3-F03 (or a variant thereof, e.g., 3-F03_411 or 3-F03_413) is linked to a heavy chain constant region comprising a CH3 domain. In some embodiments, the CH3 domain lacks the C-terminal lysine (K) amino acid residue. In some embodiments, the CH3 domain contains the C-terminal lysine (K) amino acid residue. In certain embodiments, the VH of 3-F03 (or a variant thereof, e.g., 3-F03_411 or 3-F03_413) is linked to a heavy chain constant region comprising a CH1 domain, hinge region, CH2 domain, and CH3 domain from human IgG1. In some embodiments, the CH3 domain from human IgG1 lacks the C-terminal lysine (K) amino acid residue. In some embodiments, the CH3 domain from human IgG1 contains the C-terminal lysine (K) amino acid residue. In certain embodiments such an antibody contains one or more additional mutations in the heavy chain constant region that increase the stability of the antibody. In certain embodiments, the heavy chain constant region includes substitutions that modify the properties of the antibody (e.g., decrease Fc receptor binding, increase or decrease antibody glycosylation, decrease binding to Clq).

In certain embodiments, the heavy chain constant region includes an alanine (A) at position Asparagine-297 (N297, according to EU numbering) of the heavy chain constant region to reduce effector function.

In certain embodiments, the anti-CD73 antibody is an IgG antibody. In one embodiment, the antibody is an IgG1 antibody. In one embodiment, the antibody is an IgG4 antibody. In another embodiment, the antibody is an IgG2 antibody. In certain embodiments, the anti-CD73 antibody comprises a heavy chain constant region lacking one or more lysine (K) amino acid residues relative to a wild type heavy chain constant region. For example, in certain embodiments, the antibody comprises heavy chain constant region lacking the C-terminal lysine (K) amino acid residue of the CH3 domain of the heavy chain constant region.

Additional anti-CD73 Antibodies and Inhibitors

This disclosure provides additional anti-CD73 antibodies and CD73 inhibitors that are useful in combination with an A2A and/or A2B adenosine receptor inhibitor in treating diseases, e.g., cancer. This disclosure further provides additional anti-CD73 antibodies and CD73 inhibitors that are useful in combination with an A2A and/or A2B adenosine receptor inhibitor and/or in combination with an inhibitor of PD-1/PD-L1 in treating diseases, e.g., cancer. This disclosure further provides additional anti-CD73 antibodies and CD73 inhibitors that are useful in combination with an inhibitor of PD-1/PD-L1 in treating diseases, e.g., cancer.

Other anti-CD73 antibodies useful in combination with an inhibitor of A2A and/or A2B adenosine receptor in the methods described herein are known in the art. Other anti-CD73 antibodies useful in combination with an inhibitor of A2A and/or A2B adenosine receptor and/or in combination with an inhibitor of PD-1/PD-L1 in treating diseases in the methods described herein are known in the art. Other anti-CD73 antibodies useful in combination with an inhibitor of PD-1/PD-L1 in treating diseases in the methods described herein are known in the art. See, e.g., U.S. Pat. Nos. 9,090,697, 9,388,249, 9,605,080, 9,938,356, 10,100,129, and 10,287,362, US Patent Application Publication Nos. 2004/0142342, 2007/0009518, 2011/0300136, 2018/0009899, 2018/0030144, 2018/0237536, 2018/0264107, 2019/0031766, 2019/0225703, 2019/0077873, and 2019/0256598, and international patent application publication nos. WO 2004/079013, WO 2011/089004, WO 2014/153424, WO 2017/100670, WO 2001/080884, WO 2018/110555, WO 2018/137598, WO 2018/187512, WO 2018/215535, WO 2018/237173, WO 2019/170131, WO 2019/173692, and WO 2019/173291, each of which is incorporated by reference herein in its entirety.

In some instances, the anti-CD73 antibody comprises a VH comprising a VH CDR1, a VH CDR2, and a VH CDR3 of a VH comprising the amino acid sequence EIQLQQSGPELVKPGASVKVSCKASGYAFTSYNMYWVKQSHGKSLEWIGYIDPY NGGTSYNQKFKGKATLTVDKSSSTAYMHLNSLTSEDSAVYYCARGYGNYKAW FAYWGQGTLVTVSA (SEQ ID NO:100), and a VL comprising a VL CDR1, a VL CDR2, and a VL CDR3 of a VL comprising the amino acid sequence DAVMTQTPKFLLVSAGDRVTITCKASQSVTNDVAWYQQKPGQSPKLLIYYASNR YTGVPDRFTGSGYGTDFTFTISTVQAEDLAVYFCQQDYSSLTFGAGTKLELK (SEQ ID NO:101). In some instances, the anti-CD73 antibody comprises a VH comprising the amino acid sequence set forth in SEQ ID NO:100 and a VL comprising the amino acid sequence set forth in SEQ ID NO:101. In some instances, the anti-CD73 antibody is 11E1 (see US patent application publication no. 2018/0237536, which is incorporated by reference herein in its entirety). In some instances, the anti-CD73 antibody comprises a heavy chain comprising the amino acid sequence set forth in SEQ ID NO:102. In some instances, the anti-CD73 antibody comprises a light chain comprising the amino acid sequence set forth in SEQ ID NO:103. In some instances, the anti-CD73 antibody comprises a heavy chain comprising the amino acid sequence set forth in SEQ ID NO:102 and a light chain comprising the amino acid sequence set forth in SEQ ID NO:103.

In some instances, the anti-CD73 antibody comprises a VH comprising a VH CDR1, a VH CDR2, and a VH CDR3 of a VH comprising the amino acid sequence EVQLLESGGGLVQPGGSLRLSCAASGFTFSSYAYSWVRQAPGKGLEWVSAISGS GGRTYYADSVKGRFTISRDNSKNTLYLQMNSLRAEDTAVYYCARLGYGRVDEW GRGTLVTVSS (SEQ ID NO:96), and a VL comprising a VL CDR1, a VL CDR2, and a VL CDR3 of a VL comprising the amino acid sequence QSVLTQPPSASGTPGQRVTISCSGSLSNIGRNPVNWYQQLPGTAPKLLIYLDNLRL SGVPDRFSGSKSGTSASLAISGLQSEDEADYYCATWDDSSHPGWTFGGGTKLTVL (SEQ ID NO:97). In some instances, the anti-CD73 antibody comprises a VH comprising the amino acid sequence set forth in SEQ ID NO:96 and a VL comprising the amino acid sequence set forth in SEQ ID NO:97. In some instances, the anti-CD73 antibody is Medi9447 (see U.S. Pat. No. 10,287,362, which is incorporated by reference herein in its entirety). In some instances, the anti-CD73 antibody comprises a heavy chain comprising the amino acid sequence set forth in SEQ ID NO:98. In some instances, the anti-CD73 antibody comprises a light chain comprising the amino acid sequence set forth in SEQ ID NO:99. In some instances, the anti-CD73 antibody comprises a heavy chain comprising the amino acid sequence set forth in SEQ ID NO:98 and a light chain comprising the amino acid sequence set forth in SEQ ID NO:99.

In some instances, the CD73 inhibitor is CPI-006 (Corvus; see US Patent Application Publication No. US 2018/0009899 A1 and international patent application publication no. WO 2017/100670 A1, each of which is incorporated by reference herein in its entirety).

In some instances, the CD73 inhibitor is CB-708 SM (Calithera).

In some instances, the CD73 inhibitor is AB680 (Arcus).

In some instances, the CD73 inhibitor is BMS-986179 (BMS).

### Antibody Fragments

In some instances, the anti-CD73 antibody is an antibody fragment. Fragments of the antibodies described herein (e.g., Fab, Fab′, F(ab′)₂, Facb, and Fv) may be prepared by proteolytic digestion of intact antibodies. For example, antibody fragments can be obtained by treating the whole antibody with an enzyme such as papain, pepsin, or plasmin. Papain digestion of whole antibodies produces F(ab)₂ or Fab fragments; pepsin digestion of whole antibodies yields F(ab′)₂ or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Plueckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology, 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046.

### Minibodies

In some instances, the anti-CD73 antibody is a minibody. Minibodies of anti-CD73 antibodies include diabodies, single chain (scFv), and single-chain (Fv)₂ (sc(Fv)₂). Minibodies of anti-PD-1 antibodies include diabodies, single chain (scFv), and single-chain (Fv)₂ (sc(Fv)₂).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see, e.g., Huston et al., Proc. Natl. Acad. Sci. U.S.A, 85:5879-5883 (1988); and Plickthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The heavy chain variable domain and light chain variable domain in an scFv may be derived from any anti-CD73 antibody described herein. The H chain V region and L chain V region in an scFv may be derived from any anti-PD-1 antibody or antigen-binding fragment thereof described herein.

An sc(Fv)₂ is a minibody in which two VHs and two VLs are linked by a linker to form a single chain (Hudson, et al., J Immunol. Methods, (1999) 231: 177-189 (1999)). An sc(Fv)₂ can be prepared, for example, by connecting scFvs with a linker. The sc(Fv)₂ of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order.

### Bispecific Antibodies

In some instances, the anti-CD73 antibody is a bispecific antibody. Bispecific antibodies are antibodies that have binding specificities for at least two different epitopes. Exemplary bispecific antibodies may bind to two different epitopes of the CD73 protein. Other such antibodies may combine a CD73 binding site with a binding site for another protein. Exemplary bispecific antibodies may bind to two different epitopes of the PD-1 protein. Other such antibodies may combine a PD-1 binding site with a binding site for another protein. Bispecific antibodies can be prepared as full length antibodies or low molecular weight forms thereof (e.g., F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain.

Accordingly, the VH and VL domains of one fragment are forced to pair with the complementary VL and VH domains of another fragment, thereby forming two antigen-binding sites.

### Multivalent Antibodies

In some instances, the anti-CD73 antibody is a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites.

An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

In some instances, the anti-CD73 antibody is a conjugated antibody. The antibodies disclosed herein may be conjugated antibodies, which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. ⁹⁰Y, ¹³¹I), fluorescent substances, luminescent substances, haptens, enzymes, metal chelates, drugs, and toxins (e.g., calcheamicin, Pseudomonas exotoxin A, ricin (e.g. deglycosylated ricin A chain)).

In one embodiment, to improve the cytotoxic actions of anti-CD73 antibodies and consequently their therapeutic effectiveness, the antibodies are conjugated with highly toxic substances, including radioisotopes and cytotoxic agents. In one embodiment, to improve the cytotoxic actions of anti-PD-1 antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-CD73 antibody is modified with a moiety that improves its stabilization and/or retention in circulation, e.g., in blood, serum, or other tissues, e.g., by at least 1.5, 2, 5, 10, or 50 fold. For example, the anti-CD73 antibody can be associated with (e.g., conjugated to) a polymer, e.g., a substantially non-antigenic polymer, such as a polyalkylene oxide or a polyethylene oxide. In certain embodiments, an anti-PD-1 antibody or antigen-binding fragment thereof are modified with a moiety that improves its stabilization and/or retention in circulation, e.g., in blood, serum, or other tissues, e.g., by at least 1.5, 2, 5, 10, or 50 fold. For example, the anti-PD-1 antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-CD73 antibody, the anti-PD-1 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies, respectively, or the lower molecular weight forms thereof described herein. Methods for modifying antibodies are well known in the art (e.g., U.S. Pat. Nos. 5,057,313 and 5,156,840).

### Polynucleotides, Expression Vectors, and Cells

The disclosure also provides polynucleotides and vectors encoding an anti-CD73 antibody or portion thereof (e.g., VH, VL, HC, or LC) described herein. The polynucleotides of the disclosure can be in the form of RNA or in the form of DNA. In some instances, the polynucleotide is DNA. In some instances, the polynucleotide is complementary DNA (cDNA). In some instances, the polynucleotide is RNA.

In some instances, the polynucleotide encodes a VH comprising the VH CDR1, VH CDR2, and VH CDR3 of any antibody described herein (see, e.g., Tables 3, 4, and 6). In some instances, the polynucleotide encodes a VL comprising the VL CDR1, VL CDR2, and VL CDR3 of any antibody described herein (see, e.g., Tables 3, 4, and 6). In some instances, the polynucleotide encodes a heavy chain comprising a VH comprising the VH CDR1, VH CDR2, and VH CDR3 of any antibody described herein (see, e.g., Tables 3, 4, and 6). In some instances, the polynucleotide encodes a light chain comprising a VL comprising the VL CDR1, VL CDR2, and VL CDR3 of any antibody described herein (see, e.g., Tables 3, 4, and 6). In some instances, the polynucleotide is operably linked to a promoter.

In some instances, the polynucleotide comprises: (i) a first nucleic acid sequence encoding a first polypeptide, wherein the first polypeptide comprises a VH comprising the VH CDR1, VH CDR2, and VH CDR3 of any antibody described herein (see, e.g., Tables 3, 4, and 6); and (ii) a second nucleic acid sequence encoding a second polypeptide, wherein the second polypeptide comprises a VL comprising the VL CDR1, VL CDR2, and VL CDR3 of any antibody described herein (see, e.g., Tables 3, 4, and 6).

In some instances, the polynucleotide comprises: (i) a first nucleic acid sequence encoding a first polypeptide, wherein the first polypeptide comprises a heavy chain comprising a VH comprising the VH CDR1, VH CDR2, and VH CDR3 of any antibody described herein (see, e.g., Tables 3, 4, and 6); and (ii) a second nucleic acid sequence encoding a second polypeptide, wherein the second polypeptide comprises a light chain comprising a VL comprising the VL CDR1, VL CDR2, and VL CDR3 of any antibody described herein (see, e.g., Tables 3, 4, and 6). In some instances, the first nucleic acid is operably linked to a first promoter and the second nucleic acid is operably linked to a second promoter.

In some instances, the polynucleotide encodes the VH of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:22. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84. In some instances, the polynucleotide encodes a polypeptide comprising the amino acid sequence set forth in SEQ ID NO:22. In some instances, the polynucleotide is operably linked to a promoter.

In some instances, the polynucleotide encodes the VL of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:23. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in SEQ ID NO:23. In some instances, the polynucleotide encodes a polypeptide comprising the amino acid sequence set forth in SEQ ID NO:23. In some instances, the polynucleotide is operably linked to a promoter.

In some instances, the polynucleotide encodes the VH of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:62 or 63. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in SEQ ID NO:62 or 63. In some instances, the polynucleotide encodes a polypeptide comprising the amino acid sequence set forth in SEQ ID NO:62. In some instances, the polynucleotide encodes a polypeptide comprising the amino acid sequence set forth in SEQ ID NO:63. In some instances, the polynucleotide is operably linked to a promoter.

In some instances, the polynucleotide encodes the VL of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:61. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in SEQ ID NO:61. In some instances, the polynucleotide encodes a polypeptide comprising the amino acid sequence set forth in SEQ ID NO:61. In some instances, the polynucleotide is operably linked to a promoter.

In some embodiments, a polynucleotide described herein is isolated.

Also provided herein are expression vectors encoding the anti-CD73 antibodies or portions thereof (e.g., VH, VL, HC, and/or LC) described herein. Also provided herein are expression vectors comprising one or more polynucleotides described herein. Various types of expression vectors are known in the art and described herein (e.g., see the section “Methods of Producing Antibodies” herein).

Also provided herein are cells comprising the anti-CD73 antibodies described herein. Also provided herein are cells comprising one or more polynucleotides described herein. Also provided herein are cells comprising one or more expression vectors described herein. Various types of cells are known in the art and described herein (e.g., see the section “Methods of Producing Antibodies” herein).

Anti-CD73 Antibodies with Altered Glycosylation

Different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C₁, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the microheterogeneity of human IgG oligosaccharides can affect biological functions such as CDC and ADCC, binding to various Fc receptors, and binding to Clq protein (Wright A. & Morrison S L. TIBTECH 1997, 15 26-32; Shields et al. J Biol Chem. 2001 276(9):6591-604; Shields et al. J Biol Chem. 2002; 277(30):26733-40; Shinkawa et al. J Biol Chem. 2003 278(5):3466-73; Umana et al. Nat Biotechnol. 1999 February; 17(2): 176-80). For example, the ability of IgG to bind Clq and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995). Thus, in some instances, the anti-CD73 antibody contains an Asn297Ala substitution relative to a wild type constant region.

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of aglycosyl antibodies. For example, an aglycosyl anti-CD8 antibody is incapable of depleting CD8-bearing cells in mice (Isaacs, 1992 J. Immunol. 148: 3062) and an aglycosyl anti-CD3 antibody does not induce cytokine release syndrome in mice or humans (Boyd, 1995 supra; Friend, 1999 Transplantation 68:1632). Aglycosylated forms of the anti-CD73 antibody also have reduced effector function.

Importantly, while removal of the glycans in the CH2 domain appears to have a significant effect on effector function, other functional and physical properties of the antibody remain unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

The anti-CD73 antibodies of the present invention may be modified or altered to elicit increased or decreased effector function(s) (compared to a second CD73-specific antibody). Methods for altering glycosylation sites of antibodies are described, e.g., in U.S. Pat. Nos. 6,350,861 and 5,714,350, WO 05/18572 and WO 05/03175; these methods can be used to produce anti-CD73 antibodies of the present invention with altered, reduced, or no glycosylation.

### Methods of Producing Antibodies

### Anti-CD73 Antibodies

Antibodies may be produced in bacterial or eukaryotic cells. Some antibodies, e.g., Fabs, can be produced in bacterial cells, e.g., E. coli cells. Antibodies can also be produced in eukaryotic cells such as transformed cell lines (e.g., CHO, 293E, COS). In addition, antibodies (e.g., scFvs) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-CD73 antibody (e.g., CL25, HzCL25, 3-F03, 3-F03_411, or 3-F03_413) is introduced into dhfr CHO cells by calcium phosphate-mediated transfection. In an exemplary system for antibody expression, a recombinant expression vector encoding both the antibody heavy chain and the antibody light chain of an anti-PD-1 antibody (e.g., retifanlimab) is introduced into dhfr CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Anti-PD-1 Antibodies

Anti-PD-1 antibodies, such as retifanlimab, can be made, for example, by preparing and expressing synthetic genes that encode the recited amino acid sequences or by mutating human germline genes to provide a gene that encodes the recited amino acid sequences. Moreover, this antibody and other anti-PD-1 antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237 (based on Kabat numbering). Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies may also have mutations that stabilize the disulfide bond between the two heavy chains of an immunoglobulin, such as mutations in the hinge region of IgG4, as disclosed in the art (e.g., Angal et al. (1993)Mol. Immunol. 30:105-08). See also, e.g., U.S. 2005-0037000.

The anti-PD-1 antibodies can be in the form of full length antibodies, or in the form of low molecular weight forms (e.g., biologically active antibody fragments or minibodies) of the anti-PD-1 antibodies, e.g., Fab, Fab′, F(ab′)2, Fv, Fd, dAb, scFv, and sc(Fv)₂. Other anti-PD-1 antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-PD-1 antibody or antigen-binding fragment thereof and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-PD-1 antibody or antigen-binding fragment thereof comprising at least one deamidation site, wherein the pH of the composition is from about 5.0 to about 6.5, such that, e.g., at least about 90% of the anti-PD-1 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0.

In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g. as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e. the neutral amide side chain has been converted to a residue with an overall acidic character.

The term “mixture” as used herein in reference to a composition comprising an anti-PD-1 antibody or antigen-binding fragment thereof, means the presence of both the desired anti-PD-1 antibody or antigen-binding fragment thereof and one or more acidic variants thereof. The acidic variants may comprise predominantly deamidated anti-PD-1 antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

### Dosing and Administration

The anti-CD73 antibodies, the inhibitor of A2A and/or A2B adenosine receptor, and the inhibitor of PD-1/PD-L1 described herein can be administered to a subject, e.g., a subject in need thereof, for example, a human subject, by a variety of methods. In some instances, the anti-CD73 antibodies, the inhibitor of A2A and/or A2B adenosine receptor, and the inhibitor of PD-1/PD-L1 are administered to the subject by the same route. In some instances, the anti-CD73 antibodies and the inhibitor of PD-1/PD-L1 are administered to the subject by the same route. In some instances, the anti-CD73 antibodies, the inhibitor of A2A and/or A2B adenosine receptor, and the inhibitor of PD-1/PD-L1 are administered to the subject by different routes. In some instances, the anti-CD73 antibodies and the inhibitor of PD-1/PD-L1 are administered to the subject by different routes. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the anti-CD73 antibodies, the inhibitor of A2A and/or A2B adenosine receptor, and the inhibitor of PD-1/PD-L1 can also be tailored for the individual case, e.g., by monitoring the subject, e.g., using tomographic imaging, e.g., to visualize a tumor. The route and/or mode of administration of the anti-CD73 antibodies and the inhibitor of PD-1/PD-L1 can also be tailored for the individual case, e.g., by monitoring the subject, e.g., using tomographic imaging, e.g., to visualize a tumor.

Each of the anti-CD73 antibodies, the inhibitor of A2A and/or A2B adenosine receptor, and the inhibitor of PD-1/PD-L1 can be administered as a fixed dose, or in a mg/kg patient weight dose. For example, in a dual combination treatment, each of the anti-CD73 antibodies and the inhibitor of PD-1/PD-L1 can be administered as a fixed dose, or in a mg/kg patient weight dose. The dose can also be chosen to reduce or avoid production of antibodies against the anti-CD73 antibodies, the inhibitor of A2A and/or A2B adenosine receptor, and/or the inhibitor of PD-1/PD-L1. Dosage regimens are adjusted to provide the desired response, e.g., a therapeutic response or a combinatorial therapeutic effect. Generally, doses of the anti-CD73 antibodies, the inhibitor of A2A and/or A2B adenosine receptor, and the inhibitor of PD-1/PD-L1 can be used in order to provide a subject with the agents in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used.

In some embodiments, the anti-CD73 antibodies, the inhibitor of A2A and/or A2B adenosine receptor, and/or the inhibitor of PD-1/PD-L1 are administered simultaneously. In some embodiments, the anti-CD73 antibodies and the inhibitor of PD-1/PD-L1 are administered simultaneously.

In some embodiments, the anti-CD73 antibodies, the inhibitor of A2A and/or A2B adenosine receptor, and/or the inhibitor of PD-1/PD-L1 are administered sequentially. In some embodiments, the anti-CD73 antibodies and the inhibitor of PD-1/PD-L1 are administered sequentially.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibodies and/or inhibitors may be administered via continuous infusion. Exemplary fixed doses include 375 mg, 500 mg, and 750 mg.

### A2A A2B Adenosine Receptor Inhibitors

In some embodiments, the inhibitor of A2A/A2B, or a pharmaceutically acceptable salt thereof, is administered to the subject in a dosage of from about 0.1 mg to about 1000 mg on a free base basis. In some embodiments, the inhibitor of A2A/A2B, or a pharmaceutically acceptable salt thereof, is administered to the subject in a dosage of from about 1 mg to about 500 mg on a free base basis. In some embodiments, the inhibitor of A2A/A2B, or a pharmaceutically acceptable salt thereof, is administered to the subject in a dosage of from about 5 mg to about 250 mg on a free base basis. In some embodiments, the inhibitor of A2A/A2B, or a pharmaceutically acceptable salt thereof, is administered to the subject in a dosage of from about 10 mg to about 100 mg on a free base basis.

In some embodiments, the inhibitor of A2A/A2B, or a pharmaceutically acceptable salt thereof, is administered to the subject in a dosage selected from about 0.5 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, and about 1000 mg on a free base basis. In some embodiments, the inhibitor of A2A/A2B, or a pharmaceutically acceptable salt thereof, is administered to the subject in a dosage ranging from about 0.1 mg to about 500 mg on a free base basis, or any dosage value there between. In some embodiments, the inhibitor of A2A/A2B, or a pharmaceutically acceptable salt thereof, is administered to the subject in a dosage ranging from about 1 mg to about 100 mg on a free base basis, or any dosage value there between. In some embodiments, the inhibitor of A2A/A2B is administered to the subject in a dosage of from about 0.1 mg to about 500 mg on a free base basis, wherein the inhibitor of A2A/A2B is administered once-daily or every other day.

In some embodiments, the inhibitor of A2A/A2B, or a pharmaceutically acceptable salt thereof, is administered to the subject once-daily, every other day, once-weekly or any time intervals between. In some embodiments, the inhibitor of A2A/A2B, or a pharmaceutically acceptable salt thereof, is administered to the subject once-daily. In some embodiments, the inhibitor of A2A/A2B, or a pharmaceutically acceptable salt thereof, is administered to the subject every other day. In some embodiments, the inhibitor of A2A/A2B, or a pharmaceutically acceptable salt thereof, is administered to the subject once-weekly.

In some embodiments, the inhibitor of A2A/A2B, or a pharmaceutically acceptable salt thereof, is administered to the subject in a dosage of from about 1 mg to about 50 mg QD.

In some embodiments, the inhibitor of A2A/A2B, or a pharmaceutically acceptable salt thereof, is administered to the subject in a dosage of from about 1 mg to about 50 mg BID.

In some embodiments, the inhibitor of A2A/A2B, or a pharmaceutically acceptable salt thereof, is administered to the subject in a dosage of about 10 mg QD.

In some embodiments, the inhibitor of A2A/A2B, or a pharmaceutically acceptable salt thereof, is administered to the subject in a dosage of about 10 mg BID.

In some embodiments, the inhibitor of A2A/A2B, or a pharmaceutically acceptable salt thereof, is administered to the subject in a dosage of about 40 mg QD.

In some embodiments, the inhibitor of A2A/A2B, or a pharmaceutically acceptable salt thereof, is administered to the subject in a dosage of about 40 mg BID.

In some embodiments, each of the dosages is administered as a single, once daily dosage. In some embodiments, each of the dosages is administered as a single, once daily oral dosage.

In some embodiments, the methods provided comprise administration of a first dosage of the inhibitor of A2A/A2B, or a pharmaceutically acceptable salt thereof, as defined herein, and a second dosage of the inhibitor of A2A/A2B, or a pharmaceutically acceptable salt thereof, wherein the second dosage is greater than the first dosage (i.e., the method comprises a dose escalation of the inhibitor of A2A/A2B, or a pharmaceutically acceptable salt thereof, such as Compound 9).

In some embodiments, the method comprises a dose escalation of the inhibitor of A2A/A2B, or a pharmaceutically acceptable salt thereof, when the inhibitor of A2A/A2B, or a pharmaceutically acceptable salt thereof, is administered to a subject in combination with an inhibitor of PD-1/PD-L1 and an anti-CD73 antibody, or an antigen-binding fragment thereof.

In some embodiments, the method comprises a dose escalation of Compound 9, when said Compound 9 is administered to a subject in combination with an inhibitor of PD-1/PD-L1 (e.g., retifanlimab) and in combination with an anti-CD73 antibody, or an antigen-binding fragment thereof (e.g., ANTIBODY Y).

### Inhibitor of PD-1 PD-L1

In some embodiments, the inhibitor of PD-1/PD-L1, or a pharmaceutically acceptable salt thereof, is administered to the subject in a dosage of from about 0.1 mg to about 1000 mg on a free base basis. In some embodiments, the inhibitor of PD-1/PD-L1, or a pharmaceutically acceptable salt thereof, is administered to the subject in a dosage of from about 1 mg to about 500 mg on a free base basis. In some embodiments, the inhibitor of PD-1/PD-L1, or a pharmaceutically acceptable salt thereof, is administered to the subject in a dosage of from about 5 mg to about 250 mg on a free base basis. In some embodiments, the inhibitor of PD-1/PD-L1, or a pharmaceutically acceptable salt thereof, is administered to the subject in a dosage of from about 10 mg to about 100 mg on a free base basis.

A composition may comprise about 1 mg/mL to 100 mg/mL or about 10 mg/mL to 100 mg/mL or about 50 to 250 mg/mL or about 100 to 150 mg/mL or about 100 to 250 mg/mL of the inhibitor of PD-1/PD-L1 (e.g., an anti-PD-1 antibody or antigen-binding fragment thereof).

An inhibitor of PD-1/PD-L1 (e.g., an anti-PD-1 antibody or antigen-binding fragment thereof) dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

In some embodiments, the inhibitor of PD-1/PD-L1, or a pharmaceutically acceptable salt thereof, is administered to the subject in a dosage selected from about 0.5 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, and about 1000 mg on a free base basis. In some embodiments, the inhibitor of PD-1/PD-L1, or a pharmaceutically acceptable salt thereof, is administered to the subject in a dosage ranging from about 0.1 mg to about 500 mg on a free base basis, or any dosage value there between. In some embodiments, the inhibitor of PD-1/PD-L1, or a pharmaceutically acceptable salt thereof, is administered to the subject in a dosage ranging from about 1 mg to about 100 mg on a free base basis, or any dosage value there between.

In some embodiments, the inhibitor of PD-1/PD-L1 is administered to the subject in a dosage of about 1 mg/kg to about 10 mg/kg. In some embodiments, the inhibitor of PD-1/PD-L1 is administered to the subject in a dosage of about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In some embodiments, the inhibitor of PD-1/PD-L1 is administered to the subject in a dosage of about 200 mg to about 1000 mg. In some embodiments, the inhibitor of PD-1/PD-L1 is administered to the subject in a dosage of about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg or about 1000 mg.

In some embodiments, the inhibitor of PD-1/PD-L1 is administered to the subject once-daily, every other day, once-weekly or any time intervals between. In some embodiments, the inhibitor of PD-1/PD-L1 is administered to the subject once-daily. In some embodiments, the inhibitor of PD-1/PD-L1 is administered to the subject every other day. In some embodiments, the inhibitor of PD-1/PD-L1 is administered to the subject once-weekly.

In some embodiments, each of the dosages is administered as a single, once daily dosage. In some embodiments, each of the dosages is administered as a single, once daily oral dosage.

In some embodiments, the inhibitor of PD-1/PD-L1 is administered to the subject every two weeks, every three weeks or every four weeks. In some embodiments, the inhibitor of PD-1/PD-L1 is administered to the subject monthly or quarterly. In some embodiments, the inhibitor of PD-1/PD-L1 is administered to the subject by intravenous administration.

In some embodiments, the inhibitor of PD-1/PD-L1 is administered to the subject at a dosage of 1 mg/kg Q2W.

In some embodiments, the inhibitor of PD-1/PD-L1 is administered to the subject at a dosage of 3 mg/kg Q2W.

In some embodiments, the inhibitor of PD-1/PD-L1 is administered to the subject at a dosage of 3 mg/kg Q4W.

In some embodiments, the inhibitor of PD-1/PD-L1 is administered to the subject at a dosage of 10 mg/kg Q2W.

In some embodiments, the inhibitor of PD-1/PD-L1 is administered to the subject at a dosage of 10 mg/kg Q4W.

In some embodiments, the inhibitor of PD-1/PD-L1 is administered to the subject at a dosage of 200 mg Q3W.

In some embodiments, the inhibitor of PD-1/PD-L1 is administered to the subject at a dosage of 250 mg Q3W.

In some embodiments, the inhibitor of PD-1/PD-L1 is administered to the subject at a dosage of 375 mg Q3W.

In some embodiments, the inhibitor of PD-1/PD-L1 is administered to the subject at a dosage of 500 mg Q4W.

In some embodiments, the inhibitor of PD-1/PD-L1 is administered to the subject at a dosage of 750 mg Q4W.

In some embodiments, the inhibitor of PD-1/PD-L1 is retifanlimab. In some embodiments, the retifanlimab is administered to the subject is a dosage of from about 250 mg to about to about 850 mg. In some embodiments, the retifanlimab is administered to the subject is a dosage of from about 375 mg to about to about 850 mg. In some embodiments, the retifanlimab is administered to the subject is a dosage of from about 450 mg to about to about 850 mg. In some embodiments, the retifanlimab is administered to the subject is a dosage of from about 500 mg to about to about 750 mg. In some embodiments, the retifanlimab is administered to the subject is a dosage of about 500 mg.

In some embodiments, the retifanlimab is administered to the subject is a dosage of about 750 mg. In some embodiments, the retifanlimab is administered to the subject every four weeks. In some embodiments, the retifanlimab is administered to the subject by intravenous administration.

In some embodiments, the retifanlimab is administered to the subject at a dosage of 1 mg/kg Q2W.

In some embodiments, the retifanlimab is administered to the subject at a dosage of 3 mg/kg Q2W.

In some embodiments, the retifanlimab is administered to the subject at a dosage of 3 mg/kg Q4W.

In some embodiments, the retifanlimab is administered to the subject at a dosage of 10 mg/kg Q2W.

In some embodiments, the retifanlimab is administered to the subject at a dosage of 10 mg/kg Q4W.

In some embodiments, the retifanlimab is administered to the subject at a dosage of 200 mg Q3W.

In some embodiments, the retifanlimab is administered to the subject at a dosage of 250 mg Q3W.

In some embodiments, the retifanlimab is administered to the subject at a dosage of 375 mg Q3W.

In some embodiments, the retifanlimab is administered to the subject at a dosage of 500 mg Q4W.

In some embodiments, the retifanlimab is administered to the subject at a dosage of 750 mg Q4W.

In some embodiments, the retifanlimab is administered to the subject in a dosage of about 100 mg to about 1000 mg Q4W.

In some embodiments, the methods provided comprise administration of a first dosage of the inhibitor of PD-1/PD-L1, as defined herein, and a second dosage of the inhibitor of PD-1/PD-L1, wherein the second dosage is greater than the first dosage (i.e., the method comprises a dose escalation of the inhibitor of PD-1/PD-L1, such as retifanlimab).

In some embodiments, the method comprises a dose escalation of the inhibitor of PD-1/PD-L1, when the inhibitor of PD-1/PD-L1 is administered to a subject in combination with an inhibitor of A2A and/or A2B, and/or in combination with an anti-CD73 antibody, or an antigen-binding fragment thereof.

In some embodiments, the method comprises a dose escalation of the inhibitor of PD-1/PD-L1, when the inhibitor of PD-1/PD-L1 is administered to a subject in combination with an anti-CD73 antibody, or an antigen-binding fragment thereof.

In some embodiments, the method comprises a dose escalation of retifanlimab, when said retifanlimab is administered to a subject in combination with an inhibitor of A2A and/or A2B, and/or in combination with an anti-CD73 antibody, or an antigen-binding fragment thereof.

In some embodiments, the method comprises a dose escalation of retifanlimab, when said retifanlimab is administered to a subject in combination with an anti-CD73 antibody, or an antigen-binding fragment thereof (e.g., ANTIBODY Y).

### Anti-CD73 Antibodies

In some embodiments, the anti-CD73 antibody, or antigen-binding fragment thereof, is administered to the subject in a dosage of from about 0.1 mg to about 1000 mg. In some embodiments, the anti-CD73 antibody, or antigen-binding fragment thereof, is administered to the subject in a dosage of from about 0.1 mg to about 500 mg. In some embodiments, the anti-CD73 antibody, or antigen-binding fragment thereof, is administered to the subject in a dosage of from about 0.1 mg to about 100 mg. In some embodiments, the anti-CD73 antibody, or antigen-binding fragment thereof, is administered to the subject in a dosage of from about 1 mg to about 100 mg. In some embodiments, the anti-CD73 antibody, or antigen-binding fragment thereof, is administered to the subject in a dosage of from about 50 mg to about 100 mg.

A composition may comprise about 1 mg/mL to 100 mg/mL, or about 10 mg/mL to 100 mg/mL, or about 50 to 250 mg/mL, or about 100 to 150 mg/mL, or about 100 to 250 mg/mL of the anti-CD73 antibody (e.g., the anti-CD73 antibody or antigen-binding fragment thereof). In some embodiments, the composition comprises 50 mg/mL of the anti-CD73 antibody (e.g., the anti-CD73 antibody or antigen-binding fragment thereof).

An anti-CD73 antibody dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between about 2 to 8 weeks, more preferably between about 3 to 7 weeks, and even more preferably for about 4, 5, or 6 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

In some embodiments, the inhibitor of PD-1/PD-L1, or a pharmaceutically acceptable salt thereof, is administered to the subject in a dosage selected from about 0.5 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, about 1000 mg, about 1100 mg, about 1200 mg, about 1300 mg, about 1400 mg, or about 1500 mg.

In some embodiments, the anti-CD73 antibody, or an antigen-binding fragment thereof, is administered to the subject once-daily, every other day, once-weekly, or any time intervals between. In some embodiments, the anti-CD73 antibody, or an antigen-binding fragment thereof, is administered to the subject once-daily. In some embodiments, the anti-CD73 antibody, or an antigen-binding fragment thereof, is administered to the subject every other day. In some embodiments, the anti-CD73 antibody, or an antigen-binding fragment thereof, is administered to the subject once-weekly.

In some embodiments, the anti-CD73 antibody, or an antigen-binding fragment thereof, is administered to the subject every two weeks, every three weeks, or every four weeks. In some embodiments, the anti-CD73 antibody, or an antigen-binding fragment thereof, is administered to the subject monthly or quarterly. In some embodiments, the anti-CD73 antibody, or an antigen-binding fragment thereof, is administered to the subject by intravenous administration.

In some embodiments, each of the dosages is administered as a single, once daily dosage. In some embodiments, each of the dosages is administered as a single, once daily intravenous dosage.

In some embodiments, the anti-CD73 antibody, or an antigen-binding fragment thereof, is administered to the subject in a dosage of 70 mg Q2W.

In some embodiments, the anti-CD73 antibody, or an antigen-binding fragment thereof, is administered to the subject in a dosage of 100 mg Q2W.

In some embodiments, the anti-CD73 antibody, or an antigen-binding fragment thereof, is administered to the subject in a dosage of 250 mg Q2W.

In some embodiments, the anti-CD73 antibody, or an antigen-binding fragment thereof, is administered to the subject in a dosage of 500 mg Q2W.

In some embodiments, the anti-CD73 antibody, or an antigen-binding fragment thereof, is administered to the subject in a dosage of 750 mg Q2W.

In some embodiments, the anti-CD73 antibody, or an antigen-binding fragment thereof, is administered to the subject in a dosage of 1500 mg Q2W.

In some embodiments, the anti-CD73 antibody, or an antigen-binding fragment thereof, is administered to the subject in a dosage of 70 mg Q4W.

In some embodiments, the anti-CD73 antibody, or an antigen-binding fragment thereof, is administered to the subject in a dosage of 100 mg Q4W.

In some embodiments, the anti-CD73 antibody, or an antigen-binding fragment thereof, is administered to the subject in a dosage of 250 mg Q4W.

In some embodiments, the anti-CD73 antibody, or an antigen-binding fragment thereof, is administered to the subject in a dosage of 500 mg Q4W.

In some embodiments, the anti-CD73 antibody, or an antigen-binding fragment thereof, is administered to the subject in a dosage of 750 mg Q4W.

In some embodiments, the anti-CD73 antibody, or an antigen-binding fragment thereof, is administered to the subject in a dosage of 1500 mg Q4W.

In some embodiments, the methods provided comprise administration of a first dosage of the anti-CD73 antibody, or an antigen-binding fragment thereof, as defined herein, and a second dosage of the anti-CD73 antibody, or an antigen-binding fragment thereof, wherein the second dosage is greater than the first dosage (i.e., the method comprises a dose escalation of the anti-CD73 antibody, or an antigen-binding fragment thereof, such as ANTIBODY Y).

In some embodiments, the method comprises a dose escalation of the anti-CD73 antibody, or an antigen-binding fragment thereof, when the anti-CD73 antibody, or an antigen-binding fragment thereof, is administered to a subject in combination with an inhibitor of A2A and/or A2B and/or in combination with an inhibitor of PD-1/PD-L1.

In some embodiments, the method comprises a dose escalation of the anti-CD73 antibody, or an antigen-binding fragment thereof, when the anti-CD73 antibody, or an antigen-binding fragment thereof, is administered to a subject in combination with an inhibitor of PD-1/PD-L1.

In some embodiments, the method comprises a dose escalation of ANTIBODY Y, when said ANTIBODY Y is administered to a subject in combination with an inhibitor of A2A and/or A2B and/or in combination with an inhibitor of PD-1/PD-L1.

In some embodiments, the method comprises a dose escalation of ANTIBODY Y, when said ANTIBODY Y is administered to a subject in combination with an inhibitor of PD-1/PD-L1.

### Methods of Use

The anti-CD73 antibodies of the present disclosure can modulate the activity of CD73. Accordingly, the anti-CD73 antibodies described herein can be used in methods of inhibiting CD73 by contacting CD73 with any one or more of the antibodies or compositions thereof described herein. The A2A and/or A2B inhibitors of the present disclosure can modulate the activity of A2A and/or A2B adenosine receptor. Accordingly, the A2A and/or A2B adenosine receptor inhibitors, salts or stereoisomers described herein can be used in methods of inhibiting A2A and/or A2B adenosine receptor by contacting A2A and/or A2B adenosine receptor, respectively with any one or more of the A2A and/or A2B adenosine receptor inhibitors or compositions thereof described herein. Likewise, the inhibitors of PD-1/PD-L1 of the present disclosure can modulate the activity of PD-1/PD-L1. Accordingly, the inhibitors of PD-1/PD-L1, salts or stereoisomers described herein can be used in methods of inhibiting PD-1/PD-L1 by contacting PD-1/PD-L1, respectively with any one or more of the inhibitors of PD-1/PD-L1 or compositions thereof described herein. In some embodiments, the contacting is in vivo. In some embodiments, the contacting is ex vivo or in vitro.

Another aspect of the present disclosure pertains to methods of treating a CD73-, A2A and/or A2B adenosine receptor-, and/or PD-1/PD-L1 associated disease or disorder in an individual (e.g., patient) by administering to the individual in need of such treatment a therapeutically effective amount or dose of the one or more anti-CD73 antibodies of the present disclosure, or a pharmaceutical composition thereof, a therapeutically effective amount or dose of one or more inhibitors of A2A and/or A2B adenosine receptor of the present disclosure, or a pharmaceutical composition thereof, and a therapeutically effective amount of one or more of the inhibitors of PD-1/PD-L1 of the present disclosure, or a pharmaceutical composition thereof.

Another aspect of the present disclosure pertains to methods of treating a CD73- and/or PD-1/PD-L1 associated disease or disorder in an individual (e.g., patient) by administering to the individual in need of such treatment a therapeutically effective amount or dose of the one or more anti-CD73 antibodies of the present disclosure or a pharmaceutical composition thereof, and a therapeutically effective amount of one or more of the inhibitors of PD-1/PD-L1 of the present disclosure, or a pharmaceutical composition thereof.

A CD73-associated disease or disorder can include any disease, disorder or condition that is directly or indirectly linked to expression or activity of CD73, including overexpression and/or abnormal activity levels. An A2A and/or A2B adenosine receptor-associated disease or disorder can include any disease, disorder or condition that is directly or indirectly linked to expression or activity of A2A and/or A2B adenosine receptor, including overexpression and/or abnormal activity levels. A PD-1/PD-L1-associated disease or disorder can include any disease, disorder or condition that is directly or indirectly linked to expression or activity of PD-1/PD-L1-, including overexpression and/or abnormal activity levels.

A CD73- and/or A2A and/or A2B adenosine receptor-, and/or PD-1/PD-L1 associated disease or disorder can include any disease, disorder or condition that is directly or indirectly linked to expression or activity of CD73 and/or A2A and/or A2B adenosine receptor, and/or PD-1 and/or PD-L1, including overexpression and/or abnormal activity levels of CD73 and/or A2A and/or A2B adenosine receptor, and/or PD-1, and/or PD-L1.

Another aspect of the present disclosure pertains to methods of treating a disease or disorder (e.g., cancer) in an individual (e.g., patient) by administering to the individual in need of such treatment a therapeutically effective amount or dose of the one or more anti-CD73 antibodies of the present disclosure or a pharmaceutical composition thereof and a therapeutically effective amount or dose of one or more inhibitors of A2A and/or A2B adenosine receptor of the present disclosure or a pharmaceutical composition thereof, and a therapeutically effective amount of the one or more of the inhibitors of PD-1/PD-L1 of the present disclosure, or a pharmaceutical composition thereof, wherein the disease or disorder has a high adenosine signature. Methods of determining that a disease or disorder has a high adenosine signature are known in the art. For instance, gene expression analysis of tumor tissue may be performed using a defined panel of adenosine-responsive genes.

The anti-CD73 antibodies, the inhibitors of A2A and/or A2B adenosine receptor, and the inhibitors of PD-1/PD-L1 of the present disclosure can function synergistically, e.g., to treat a disease or disorder, e.g., cancer. For example, in a dual combination treatment, the anti-CD73 antibodies and the inhibitors of PD-1/PD-L1 of the present disclosure can function synergistically, e.g., to treat a disease or disorder, e.g., cancer. Accordingly, the anti-CD73 antibodies, the inhibitors of A2A and/or A2B adenosine receptor, and the inhibitors of PD-1/PD-L1 described herein can be used in combination in methods of inhibiting CD73, A2A and/or A2B adenosine receptor, and/or PD-1/PD-L1 by contacting the CD73 with any one or more of the anti-CD73 antibodies or compositions thereof described herein, contacting A2A and/or A2B adenosine receptor with any one or more of the inhibitors of A2A and/or A2B adenosine receptor or compositions thereof described herein, and contacting the PD-1/PD-L1 with any one or more of the inhibitors of PD-1/PD-L1, or compositions thereof described herein. In some embodiments, the anti-CD73 antibodies and the inhibitors of PD-1/PD-L1 described herein can be used in combination in methods of inhibiting CD73 and/or PD-1/PD-L1 by contacting the CD73 with any one or more of the anti-CD73 antibodies or compositions thereof described herein and contacting the PD-1/PD-L1 with any one or more of the inhibitors of PD-1/PD-L1, or compositions thereof described herein.

The anti-CD73 antibodies, the inhibitors of A2A and/or A2B adenosine receptor, and the inhibitors of PD-1/PD-L1 of the present disclosure are useful in combination in the treatment of diseases related to the activity of CD73 and/or A2A and/or A2B adenosine receptor, and/or PD-1/PD-L1 including, for example, cancer, inflammatory diseases, cardiovascular diseases, neurodegenerative diseases, immunomodulatory disorders, central nerve system diseases, and diabetes. The anti-CD73 antibodies and the inhibitors of PD-1/PD-L1 of the present disclosure are useful in combination in the treatment of diseases related to the activity of CD73 and/or PD-1/PD-L1 including, for example, cancer, inflammatory diseases, cardiovascular diseases, neurodegenerative diseases, immunomodulatory disorders, central nerve system diseases, and diabetes.

Based on the compelling roles of CD73, A2A and/or A2B adenosine receptor, and/or PD-1/PD-L1 in multiple immunosuppressive mechanisms, combination therapy can boost the immune system to suppress tumor progression. Anti-CD73 antibodies, inhibitors of A2A and/or A2B adenosine receptor, and inhibitors of PD-1/PD-L1 can be used in combination to treat, optionally in further combination with other therapies, bladder cancer, lung cancer (e.g., non-small cell lung cancer (NSCLC), lung metastasis), melanoma (e.g., metastatic melanoma), breast cancer (e.g., breast adenocarcinoma), cervical cancer, ovarian cancer, colorectal cancer, pancreatic cancer, esophageal cancer, prostate cancer, kidney cancer, skin cancer, thyroid cancer, liver cancer (e.g., hepatocellular carcinoma), uterine cancer, head and neck cancer (e.g., head and neck squamous cell carcinoma), and renal cell carcinoma.

Examples of cancers that are treatable using the treatment methods and regimens of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The methods of the present disclosure are also useful for the treatment of metastatic cancers, especially metastatic cancers that express PD-L1.

In some embodiments, cancers treatable with methods of the present disclosure include melanoma (e.g., metastatic malignant melanoma), renal cancer (e.g. clear cell carcinoma), prostate cancer (e.g. hormone refractory prostate adenocarcinoma), breast cancer (e.g., breast adenocarcinoma), colon cancer, lung cancer (e.g. non-small cell lung cancer and small cell lung cancer), squamous cell head and neck cancer, urothelial cancer (e.g. bladder) and cancers with high microsatellite instability (MSIhigh). Additionally, the disclosure includes refractory or recurrent malignancies whose growth may be inhibited using the methods of the disclosure.

In some embodiments, cancers that are treatable using the methods of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer (e.g., breast adenocarcinoma), lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the methods of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the cancer is selected from lung cancer (e.g., non-small cell lung cancer), melanoma, pancreatic cancer, breast cancer (e.g., breast adenocarcinoma), prostate cancer, liver cancer, colon cancer, endometrial cancer, bladder cancer, skin cancer, cancer of the uterus, ovarian cancer, cancer of the head or neck, thyroid cancer, renal cancer, gastric cancer, and sarcoma. In some embodiments, the cancer is selected from acute lymphoblastic leukemia, acute myelogenous leukemia, chronic lymphocytic leukemia, chronic myelogenous leukemia, diffuse large-B cell lymphoma, mantle cell lymphoma, non-Hodgkin lymphoma, Hodgkin lymphoma, multiple myeloma, polycythemia vera, essential thrombocythemia, chronic myelogenous leukemia, myelofibrosis, primary myelofibrosis, post-polycythemia vera/essential thrombocythemia myelofibrosis, post-essential thrombocythemia myelofibrosis and post-polycythemia vera myelofibrosis. In some embodiments, the cancer is selected from melanoma, endometrial cancer, lung cancer, renal cell carcinoma, urothelial carcinoma, bladder cancer, breast cancer (e.g., breast adenocarcinoma), and pancreatic cancer.

In some embodiments, the cancer is selected from bladder cancer, lung cancer (e.g., non-small cell lung cancer (NSCLC), small cell lung cancer, or lung metastasis), melanoma (e.g., metastatic melanoma), breast cancer (e.g., breast adenocarcinoma), cervical cancer, ovarian cancer, colon cancer, rectal cancer, colorectal cancer, pancreatic cancer, esophageal cancer, prostate cancer, kidney cancer, skin cancer, thyroid cancer, liver cancer, uterine cancer, head and neck cancer, renal cell carcinoma, endometrial cancer, anal cancer, cholangiocarcinoma, oral cancer, non-melanoma skin cancer, and Merkel call carcinoma.

In some embodiments, the prostate cancer is metastatic castrate-resistant prostate carcinoma (mCRPC).

In some embodiments, the colorectal cancer is colorectal carcinoma (CRC).

In some embodiments, the disease or disorder is lung cancer (e.g., non-small cell lung cancer), melanoma, pancreatic cancer, breast cancer (e.g., breast adenocarcinoma), head and neck squamous cell carcinoma, prostate cancer, liver cancer, color cancer, endometrial cancer, bladder cancer, skin cancer, cancer of the uterus, renal cancer, gastric cancer, or sarcoma. In some embodiments, the sarcoma is Askin's tumor, sarcoma botryoides, chondrosarcoma, Ewing's sarcoma, malignant hemangioendothelioma, malignant schwannoma, osteosarcoma, alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, rhabdomyosarcoma, synovial sarcoma, or undifferentiated pleomorphic sarcoma.

In some embodiments, the disease or disorder is head and neck cancer (e.g., head and neck squamous cell carcinoma), colorectal cancer, lung cancer (e.g., non-small cell lung cancer (NSCLC)), melanoma, ovarian, bladder, liver cancer (e.g., hepatocellular carcinoma), or renal cell carcinoma.

In some embodiments, the cancer is mesothelioma or adrenocarcinoma. In some embodiments, the disease or disorder is mesothelioma. In some embodiments, the cancer is adrenocarcinoma.

MDSC (myeloid-derived suppressor cells) are a heterogenous group of immune cells from the myeloid lineage (a family of cells that originate from bone marrow stem cells). MDSCs strongly expand in pathological situations such as chronic infections and cancer, as a result of an altered haematopoiesis. MDSCs are discriminated from other myeloid cell types in which they possess strong immunosuppressive activities rather than immunostimulatory properties. Similar to other myeloid cells, MDSCs interact with other immune cell types including T cells, dendritic cells, macrophages and natural killer cells to regulate their functions. In some embodiments, the compounds, etc. described herein can be used in methods related to cancer tissue (e.g., tumors) with high infiltration of MDSCs, including solid tumors with high basal level of macrophage and/or MDSC infiltration. In some embodiments, the combination therapy described herein can be used in methods related to cancer tissue (e.g., tumors) with tumor or tumor infiltrating lymphocytes (TILs) that express PD-1 or PD-L1.

In some embodiments, the cancer is neck and head cancer, lung cancer, ovarian cancer, prostate cancer, breast cancer, bladder cancer, colorectal cancer, gastric cancer, gastroesophageal cancer (e.g., gastroesophageal junction cancer), anal cancer, liver cancer, or pancreatic cancer.

In some embodiments, the cancer is neck and head cancer, lung cancer, ovarian cancer, prostate cancer, breast cancer, bladder cancer, colorectal cancer, or pancreatic cancer.

In some embodiments, the cancer is squamous cell carcinoma of the neck and head (SCCNH), non-small cell lung cancer (NSCLC), ovarian cancer, castration-resistant prostate cancer (CRPC), triple-negative breast cancer (TNBC), bladder cancer, metastatic colorectal cancer (mCRC), pancreatic ductal adenocarcinoma (PDAC), gastric/gastroesophageal junction (GEJ) cancer, hepatocellular carcinoma (HCC), or squamous carcinoma of the anal canal (SCAC).

In some embodiments, the cancer is squamous cell carcinoma of the neck and head (SCCNH), non-small cell lung cancer (NSCLC), ovarian cancer, castration-resistant prostate cancer (CRPC), triple-negative breast cancer (TNBC), bladder cancer, metastatic colorectal cancer (mCRC), or pancreatic cancer.

In some embodiments, the cancer is head and neck squamous cell carcinoma (HNSCC), non-small cell lung cancer (NSCLC), colorectal cancer (e.g., colon cancer), melanoma, ovarian cancer, bladder cancer, renal cell carcinoma, liver cancer, or hepatocellular carcinoma.

In some embodiments, the cancer is neck and head cancer.

In some embodiments, the cancer is squamous cell carcinoma of the neck and head (SCCNH).

In some embodiments, the cancer is lung cancer.

In some embodiments, the cancer is non-small cell lung cancer (NSCLC).

In some embodiments, the cancer is ovarian cancer.

In some embodiments, the cancer is prostate cancer.

In some embodiments, the cancer is castration-resistant prostate cancer (CRPC).

In some embodiments, the cancer is breast cancer.

In some embodiments, the cancer is triple-negative breast cancer (TNBC).

In some embodiments, the cancer is bladder cancer.

In some embodiments, the cancer is colorectal cancer.

In some embodiments, the cancer is metastatic colorectal cancer (mCRC).

In some embodiments, the cancer is pancreatic cancer.

In some embodiments, the cancer is gastric cancer.

In some embodiments, the cancer is gastroesophageal cancer.

In some embodiments, the cancer is gastric gastroesophageal junction (GEJ) cancer.

In some embodiments, the cancer is hepatocellular carcinoma (HCC).

In some embodiments, the cancer is pancreatic ductal adenocarcinoma (PDAC).

In some embodiments, the cancer is squamous carcinoma of the anal canal (SCAC).

In some embodiments, the cancer is selected from bladder cancer, breast cancer (e.g., breast adenocarcinoma), cervical cancer, colon cancer, rectal cancer, colorectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, renal cell carcinoma, and Merkel cell carcinoma.

In some embodiments, the cancer is selected from bladder cancer, breast cancer (e.g., breast adenocarcinoma), cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma.

In some embodiments, the cancer is selected from melanoma, endometrial cancer, lung cancer, kidney cancer, bladder cancer, breast cancer (e.g., breast adenocarcinoma), pancreatic cancer, colon cancer, head and neck cancer, colorectal cancer, ovarian, liver cancer, or renal cell carcinoma.

In some embodiments, the cancer is selected from the cancer is selected from melanoma, endometrial cancer, lung cancer, kidney cancer, bladder cancer, breast cancer (e.g., breast adenocarcinoma), pancreatic cancer, and colon cancer.

In some embodiments, the cancer is selected from endometrial cancer, anal cancer, and cholangiocarcinoma.

In some embodiments, the cancer is a tumor that displays high adenosine levels in the tumor microenvironment. These tumors may be enriched by a gene expression signature, or enriched by high expression levels of CD73 and/or other alkaline phosphatases, including tissue nonspecific alkaline phosphatase (i.e., TNAP and PAP).

In some embodiments, the cancer is colon cancer. In some embodiments, the cancer is melanoma. In some embodiments, the cancer is endometrial cancer. In some embodiments, the endometrial cancer is endometrioid adenocarcinoma. In some embodiments, the cancer is lung cancer. In some embodiments, the lung cancer is selected from non-small cell lung cancer and small cell lung cancer. In some embodiments, the cancer is renal cell carcinoma. In some embodiments, the cancer is urothelial carcinoma. In some embodiments, the cancer is bladder cancer. In some embodiments, the cancer is breast cancer. In some embodiments, the breast cancer is breast adenocarcinoma. In some embodiments, the breast cancer is triple-negative breast cancer. In some embodiments, the cancer is pancreatic cancer. In some embodiments, the pancreatic cancer is pancreatic ductal adenocarcinoma. In some embodiments, the cancer is a sarcoma. In some embodiments, the sarcoma is selected from Askin's tumor, sarcoma botryoides, chondrosarcoma, Ewing's sarcoma, malignant hemangioendothelioma, malignant schwannoma, osteosarcoma, alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, rhabdomyosarcoma, synovial sarcoma, and undifferentiated pleomorphic sarcoma.

In some embodiments, the anti-CD73 antibodies, the inhibitors of A2A and/or A2B adenosine receptor, and the inhibitors of PD-1/PD-L1 of the disclosure can be used in combination in treating pulmonary inflammation, including bleomycin-induced pulmonary fibrosis and injury related to adenosine deaminase deficiency. In some embodiments, the anti-CD73 antibodies and the inhibitors of PD-1/PD-L1 of the disclosure can be used in combination in treating pulmonary inflammation, including bleomycin-induced pulmonary fibrosis and injury related to adenosine deaminase deficiency.

In some embodiments, anti-CD73 antibodies, the inhibitors of A2A and/or A2B adenosine receptor, and the inhibitors of PD-1/PD-L1 of the disclosure can be used in combination as a treatment for inflammatory disease such as allergic reactions (e.g., CD73- and/or A2A and/or A2B adenosine receptor-, and/or PD-1/PD-L1 dependent allergic reactions) and other CD73- and/or A2A and/or A2B adenosine receptor, and/or PD-1/PD-L1-immune reactions. Further inflammatory diseases that can be treated by combination of the anti-CD73 antibodies, the inhibitors of A2A and/or A2B adenosine receptor, and the inhibitors of PD-1/PD-L1 inhibitors of the disclosure include respiratory disorders, sepsis, reperfusion injury, and thrombosis.

In some embodiments, anti-CD73 antibodies and the inhibitors of PD-1/PD-L1 of the disclosure can be used in combination as a treatment for inflammatory disease such as allergic reactions (e.g., CD73- and/or PD-1/PD-L1 dependent allergic reactions) and other CD73- and/or PD-1/PD-L1-immune reactions. Further inflammatory diseases that can be treated by combination of the anti-CD73 antibodies and the inhibitors of PD-1/PD-L1 inhibitors of the disclosure include respiratory disorders, sepsis, reperfusion injury, and thrombosis.

In some embodiments, the anti-CD73 antibodies, the inhibitors of A2A and/or A2B adenosine receptor, and the inhibitors of PD-1/PD-L1 of the disclosure can be used in combination as a treatment for cardiovascular disease such as coronary artery disease (myocardial infarction, angina pectoris, heart failure), cerebrovascular disease (stroke, transient ischemic attack), peripheral artery disease, and aortic atherosclerosis and aneurysm. Atherosclerosis is an underlying etiologic factor in many types of cardiovascular disease. Atherosclerosis begins in adolescence with fatty streaks, which progress to plaques in adulthood and finally results in thrombotic events that cause occlusion of vessels leading to clinically significant morbidity and mortality.

In some embodiments, the anti-CD73 antibodies and the inhibitors of PD-1/PD-L1 of the disclosure can be used in combination as a treatment for cardiovascular disease such as coronary artery disease (myocardial infarction, angina pectoris, heart failure), cerebrovascular disease (stroke, transient ischemic attack), peripheral artery disease, and aortic atherosclerosis and aneurysm. Atherosclerosis is an underlying etiologic factor in many types of cardiovascular disease. Atherosclerosis begins in adolescence with fatty streaks, which progress to plaques in adulthood and finally results in thrombotic events that cause occlusion of vessels leading to clinically significant morbidity and mortality.

In some embodiments, the anti-CD73 antibodies, the inhibitors of A2A and/or A2B adenosine receptor, and the inhibitors of PD-1/PD-L1 of the disclosure can be used in combination as a treatment for disorders in motor activity; deficiency caused by degeneration of the striatonigral dopamine system; Parkinson's disease; and some of the motivational symptoms of depression.

In some embodiments, the anti-CD73 antibodies and the inhibitors of PD-1/PD-L1 of the disclosure can be used in combination as a treatment for disorders in motor activity; deficiency caused by degeneration of the striatonigral dopamine system; Parkinson's disease; and some of the motivational symptoms of depression.

In some embodiments, the anti-CD73 antibodies, the inhibitors of A2A and/or A2B adenosine receptor, and the inhibitors of PD-1/PD-L1 of the disclosure can be used in combination as a treatment for diabetes and related disorders, such as insulin resistance. Diabetes affects the production of adenosine and the expression of A2B adenosine receptors (A2BRs) that stimulate IL-6 and CRP production, insulin resistance, and the association between A2BR gene single-nucleotide polymorphisms (ADORA2B SNPs) and inflammatory markers. The increased A2BR signaling in diabetes may increase insulin resistance in part by elevating pro-inflammatory mediators. Selective CD73 inhibitors may be useful to treat insulin resistance.

In some embodiments, the anti-CD73 antibodies and the inhibitors of PD-1/PD-L1 of the disclosure can be used in combination as a treatment for diabetes and related disorders, such as insulin resistance.

In some embodiments, the present application further provides a method of treating a cancer selected from bladder cancer, breast cancer (e.g., breast adenocarcinoma tumor), cervical cancer, colon cancer, rectal cancer, colorectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, renal cell carcinoma, and Merkel cell carcinoma in a subject, comprising administering to the subject:

(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof;

(ii) an inhibitor of PD-1/PD-L1, which is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:

the VH CDR1 comprises the amino acid sequence SYWMN (SEQ ID NO:6);

the VH CDR2 comprises the amino acid sequence VIHPSDSETWLDQKFKD (SEQ ID NO:7); and

the VH CDR3 comprises the amino acid sequence EHYGTSPFAY (SEQ ID NO:8); and

wherein the antibody comprises a variable light (VL) domain comprising VL CDR1, VL CDR2, and VL CDR3, wherein:

the VL CDR1 comprises the amino acid sequence RASESVDNYGMSFMNW (SEQ ID NO:9);

the VL CDR2 comprises the amino acid sequence AASNQGS (SEQ ID NO:10); and

the VL CDR3 comprises the amino acid sequence QQSKEVPYT (SEQ ID NO:11); and

(iii) an antibody that binds to human CD73, wherein the antibody that binds to human CD73:

(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO:16);
the VH CDR2 comprises the amino acid sequence IYPGSGNT (SEQ ID NO:17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the present application provides a method of treating breast cancer (e.g., breast adenocarcinoma tumor) in a subject, comprising administering to the subject:

(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof;

(ii) an inhibitor of PD-1/PD-L1, which is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:

the VH CDR1 comprises the amino acid sequence SYWMN (SEQ ID NO:6);

the VH CDR2 comprises the amino acid sequence VIHPSDSETWLDQKFKD (SEQ ID NO:7); and

the VH CDR3 comprises the amino acid sequence EHYGTSPFAY (SEQ ID NO:8); and

wherein the antibody comprises a variable light (VL) domain comprising VL CDR1, VL CDR2, and VL CDR3, wherein:

the VL CDR1 comprises the amino acid sequence RASESVDNYGMSFMNW (SEQ ID NO:9);

the VL CDR2 comprises the amino acid sequence AASNQGS (SEQ ID NO:10); and

the VL CDR3 comprises the amino acid sequence QQSKEVPYT (SEQ ID NO:11); and

(iii) an antibody that binds to human CD73, wherein the antibody that binds to human CD73:

(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO:17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the present application further provides a method of treating a cancer selected from bladder cancer, breast cancer (e.g., breast adenocarcinoma tumor), cervical cancer, colon cancer, rectal cancer, colorectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, renal cell carcinoma, and Merkel cell carcinoma in a subject, comprising administering to the subject:

(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof;

(ii) an inhibitor of PD-1/PD-L1, which is retifanlimab; and

(iii) an antibody that binds to human CD73, which is ANTIBODY Y.

In some embodiments, the present application provides a method of treating breast cancer (e.g., breast adenocarcinoma tumor) in a subject, comprising administering to the subject:

(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof;

(ii) an inhibitor of PD-1/PD-L1, which is retifanlimab; and

(iii) an antibody that binds to human CD73, which is ANTIBODY Y.

In some embodiments, the present application further provides a method of treating a cancer selected from bladder cancer, breast cancer (e.g., breast adenocarcinoma tumor), cervical cancer, colon cancer, rectal cancer, colorectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, renal cell carcinoma, and Merkel cell carcinoma in a subject, comprising administering to the subject:

(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof;

(ii) an inhibitor of PD-1/PD-L1, which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof; and

(iii) an antibody that binds to human CD73, wherein the antibody that binds to human CD73:

(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO: 17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the present application provides a method of treating breast cancer (e.g., breast adenocarcinoma tumor) in a subject, comprising administering to the subject:

(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof;

(ii) an inhibitor of PD-1/PD-L1, which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof; and

(iii) an antibody that binds to human CD73, wherein the antibody that binds to human CD73:

(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO:17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the present application further provides a method of treating a cancer selected from bladder cancer, breast cancer (e.g., breast adenocarcinoma tumor), cervical cancer, colon cancer, rectal cancer, colorectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, renal cell carcinoma, and Merkel cell carcinoma in a subject, comprising administering to the subject:

(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof;

(ii) an inhibitor of PD-1/PD-L1, which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof; and

(iii) an antibody that binds to human CD73, which is ANTIBODY Y.

In some embodiments, the present application provides a method of treating breast cancer (e.g., breast adenocarcinoma tumor) in a subject, comprising administering to the subject:

(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof;

(ii) an inhibitor of PD-1/PD-L1, which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof; and

(iii) an antibody that binds to human CD73, which is ANTIBODY Y.

In some embodiments, the present application further provides a method of treating a cancer selected from neck and head cancer, lung cancer, ovarian cancer, prostate cancer, breast cancer, bladder cancer, colorectal cancer, gastric cancer, gastroesophageal junction cancer, anal cancer, liver cancer, or pancreatic cancer in a subject, comprising administering to the subject:

(i) an inhibitor of PD-1/PD-L1, which is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:

the VH CDR1 comprises the amino acid sequence SYWMN (SEQ ID NO:6);

the VH CDR2 comprises the amino acid sequence VIHPSDSETWLDQKFKD (SEQ ID NO:7); and

the VH CDR3 comprises the amino acid sequence EHYGTSPFAY (SEQ ID NO:8); and

wherein the antibody comprises a variable light (VL) domain comprising VL CDR1, VL CDR2, and VL CDR3, wherein:

the VL CDR1 comprises the amino acid sequence RASESVDNYGMSFMNW (SEQ ID NO:9);

the VL CDR2 comprises the amino acid sequence AASNQGS (SEQ ID NO:10); and

the VL CDR3 comprises the amino acid sequence QQSKEVPYT (SEQ ID NO:11); and

(ii) an antibody that binds to human CD73, wherein the antibody that binds to human CD73:

(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO:17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the present application provides a method of treating a cancer selected from squamous cell carcinoma of the neck and head (SCCNH), non-small cell lung cancer (NSCLC), ovarian cancer, castration-resistant prostate cancer (CRPC), triple-negative breast cancer (TNBC), bladder cancer, metastatic colorectal cancer (mCRC), pancreatic ductal adenocarcinoma (PDAC), gastric/gastroesophageal junction (GEJ) cancer, hepatocellular carcinoma (HCC), and squamous carcinoma of the anal canal (SCAC) in a subject, comprising administering to the subject:

(i) an inhibitor of PD-1/PD-L1, which is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:

the VH CDR1 comprises the amino acid sequence SYWMN (SEQ ID NO:6);

the VH CDR2 comprises the amino acid sequence VIHPSDSETWLDQKFKD (SEQ ID NO:7); and

the VH CDR3 comprises the amino acid sequence EHYGTSPFAY (SEQ ID NO:8); and

wherein the antibody comprises a variable light (VL) domain comprising VL CDR1, VL CDR2, and VL CDR3, wherein:

the VL CDR1 comprises the amino acid sequence RASESVDNYGMSFMNW (SEQ ID NO:9);

the VL CDR2 comprises the amino acid sequence AASNQGS (SEQ ID NO:10); and

the VL CDR3 comprises the amino acid sequence QQSKEVPYT (SEQ ID NO:11); and

(ii) an antibody that binds to human CD73, wherein the antibody that binds to human CD73:

(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO:17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the present application further provides a method of treating a cancer selected from neck and head cancer, lung cancer, ovarian cancer, prostate cancer, breast cancer, bladder cancer, colorectal cancer, gastric cancer, gastroesophageal junction cancer, anal cancer, liver cancer, and pancreatic cancer in a subject, comprising administering to the subject:

(i) an inhibitor of PD-1/PD-L1, which is retifanlimab; and

(ii) an antibody that binds to human CD73, which is ANTIBODY Y.

In some embodiments, the present application provides a method of treating a cancer selected from squamous cell carcinoma of the neck and head (SCCNH), non-small cell lung cancer (NSCLC), ovarian cancer, castration-resistant prostate cancer (CRPC), triple-negative breast cancer (TNBC), bladder cancer, metastatic colorectal cancer (mCRC), and pancreatic cancer in a subject, comprising administering to the subject:

(i) an inhibitor of PD-1/PD-L1, which is retifanlimab; and

(ii) an antibody that binds to human CD73, which is ANTIBODY Y.

In some embodiments, the present application further provides a method of treating a cancer selected from neck and head cancer, lung cancer, ovarian cancer, prostate cancer, breast cancer, bladder cancer, colorectal cancer, and pancreatic cancer in a subject, comprising administering to the subject:

(i) an inhibitor of PD-1/PD-L1, which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof; and

(ii) an antibody that binds to human CD73, wherein the antibody that binds to human CD73:

(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO:16);
the VH CDR2 comprises the amino acid sequence IYPGSGNT (SEQ ID NO:17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the present application provides a method of treating a cancer selected from squamous cell carcinoma of the neck and head (SCCNH), non-small cell lung cancer (NSCLC), ovarian cancer, castration-resistant prostate cancer (CRPC), triple-negative breast cancer (TNBC), bladder cancer, metastatic colorectal cancer (mCRC), pancreatic ductal adenocarcinoma (PDAC), gastric/gastroesophageal junction (GEJ) cancer, hepatocellular carcinoma (HCC), and squamous carcinoma of the anal canal (SCAC) in a subject, comprising administering to the subject:

(i) an inhibitor of PD-1/PD-L1, which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof; and

(ii) an antibody that binds to human CD73, wherein the antibody that binds to human CD73:

(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO: 17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the present application further provides a method of treating a cancer selected from neck and head cancer, lung cancer, ovarian cancer, prostate cancer, breast cancer, bladder cancer, colorectal cancer, gastric cancer, gastroesophageal junction cancer, anal cancer, liver cancer, and pancreatic cancer in a subject, comprising administering to the subject:

(i) an inhibitor of PD-1/PD-L1, which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof; and

(ii) an antibody that binds to human CD73, which is ANTIBODY Y.

In some embodiments, the present application provides a method of treating a cancer selected from squamous cell carcinoma of the neck and head (SCCNH), non-small cell lung cancer (NSCLC), ovarian cancer, castration-resistant prostate cancer (CRPC), triple-negative breast cancer (TNBC), bladder cancer, metastatic colorectal cancer (mCRC), pancreatic ductal adenocarcinoma (PDAC), gastric/gastroesophageal junction (GEJ) cancer, hepatocellular carcinoma (HCC), and squamous carcinoma of the anal canal (SCAC) in a subject, comprising administering to the subject:

(i) an inhibitor of PD-1/PD-L1, which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof; and

(ii) an antibody that binds to human CD73, which is ANTIBODY Y.

In some embodiments of administering the inhibitor of PD-1/PD-L1 and antibody that binds to human CD73, the cancer is neck and head cancer, lung cancer, ovarian cancer, prostate cancer, breast cancer, bladder cancer, colorectal cancer, or pancreatic cancer.

In some embodiments of administering the inhibitor of PD-1/PD-L1 and antibody that binds to human CD73, the cancer is head and neck squamous cell carcinoma (HNSCC), non-small cell lung cancer (NSCLC), colorectal cancer, melanoma, ovarian cancer, bladder cancer, renal cell carcinoma, or hepatocellular carcinoma.

In some embodiments of administering the inhibitor of PD-1/PD-L1 and antibody that binds to human CD73, the cancer is neck and head cancer.

In some embodiments of administering the inhibitor of PD-1/PD-L1 and antibody that binds to human CD73, the cancer is squamous cell carcinoma of the neck and head (SCCNH).

In some embodiments of administering the inhibitor of PD-1/PD-L1 and antibody that binds to human CD73, the cancer is lung cancer.

In some embodiments of administering the inhibitor of PD-1/PD-L1 and antibody that binds to human CD73, the cancer is non-small cell lung cancer (NSCLC).

In some embodiments of administering the inhibitor of PD-1/PD-L1 and antibody that binds to human CD73, the cancer is ovarian cancer.

In some embodiments of administering the inhibitor of PD-1/PD-L1 and antibody that binds to human CD73, the cancer is prostate cancer.

In some embodiments of administering the inhibitor of PD-1/PD-L1 and antibody that binds to human CD73, the cancer is castration-resistant prostate cancer (CRPC).

In some embodiments of administering the inhibitor of PD-1/PD-L1 and antibody that binds to human CD73, the cancer is breast cancer.

In some embodiments of administering the inhibitor of PD-1/PD-L1 and antibody that binds to human CD73, the cancer is triple-negative breast cancer (TNBC).

In some embodiments of administering the inhibitor of PD-1/PD-L1 and antibody that binds to human CD73, the cancer is bladder cancer.

In some embodiments of administering the inhibitor of PD-1/PD-L1 and antibody that binds to human CD73, the cancer is colorectal cancer.

In some embodiments of administering the inhibitor of PD-1/PD-L1 and antibody that binds to human CD73, the cancer is metastatic colorectal cancer (mCRC).

In some embodiments of administering the inhibitor of PD-1/PD-L1 and antibody that binds to human CD73, the cancer is pancreatic cancer.

In some embodiments of administering the inhibitor of PD-1/PD-L1 and antibody that binds to human CD73, the cancer is gastric cancer.

In some embodiments of administering the inhibitor of PD-1/PD-L1 and antibody that binds to human CD73, the cancer is gastroesophageal cancer.

In some embodiments of administering the inhibitor of PD-1/PD-L1 and antibody that binds to human CD73, the cancer is gastric/gastroesophageal junction (GEJ) cancer.

In some embodiments of administering the inhibitor of PD-1/PD-L1 and antibody that binds to human CD73, the cancer is hepatocellular carcinoma (HCC).

In some embodiments of administering the inhibitor of PD-1/PD-L1 and antibody that binds to human CD73, the cancer is pancreatic ductal adenocarcinoma (PDAC).

In some embodiments of administering the inhibitor of PD-1/PD-L1 and antibody that binds to human CD73, the cancer is squamous carcinoma of the anal canal (SCAC).

As used herein, the term “contacting” refers to the bringing together of indicated moieties in an in vitro system or an in vivo system. For example, “contacting” A2A/A2B with a compound described herein includes the administration of a compound of the present invention to an individual or patient, such as a human, having an A2A/A2B, as well as, for example, introducing a compound described herein into a sample containing a cellular or purified preparation containing the A2A/A2B.

The terms “individual” or “patient” or “subject”, used interchangeably, refer to any animal, including mammals, preferably mice, rats, other rodents, rabbits, dogs, cats, swine, cattle, sheep, horses, or primates, and most preferably humans (i.e., a human subject).

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, “QD” is taken to mean a dosage administered to the subject once-daily. “QOD” is taken to mean a dosage administered to the subject once, every other day. “QW” is taken to mean a dosage administered to the subject once-weekly. “Q2W” is taken to mean a dosage administered to the subject once, every other week. “Q3W” is taken to mean a dosage administered to the subject once, every three weeks. “Q4W” is taken to mean a dosage administered to the subject once, every four weeks.

In some embodiments, the anti-CD73 antibodies, the inhibitors of A2A and/or A2B adenosine receptor, and the inhibitors of PD-1/PD-L1 of the disclosure are useful in combination in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Pharmaceutical Composition and Formulations

The anti-CD73 antibodies, the inhibitors of A2A and/or A2B adenosine receptor, and the inhibitors of PD-1/PD-L1 described herein can be formulated as pharmaceutical compositions for administration to a subject, e.g., to treat a disorder described herein. In some instances, the pharmaceutical composition comprises an anti-CD73 antibody as a single agent. In some instances, the pharmaceutical composition comprises an inhibitor of A2A and/or A2B adenosine receptor as a single agent.

In some instances, the pharmaceutical composition comprises an inhibitor of PD-1/PD-L1 as a single agent. In some instances, the pharmaceutical composition comprises one or more of the anti-CD73 antibodies, the inhibitors of A2A and/or A2B adenosine receptor, and the inhibitors of PD-1/PD-L1 described herein. In some instances, the pharmaceutical composition comprises one or more of the anti-CD73 antibodies and the inhibitors of PD-1/PD-L1 described herein.

When employed as pharmaceuticals, the compounds of the disclosure can be administered in the form of pharmaceutical compositions. These compositions can be prepared in a manner well known in the pharmaceutical art and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

A pharmaceutical composition may include a “therapeutically effective amount” of an agent described herein. Such effective amounts can be determined based on the effect of the administered agent or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the one or more active ingredients are typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the one or more active ingredients can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration.

The compositions of the disclosure can further include one or more additional pharmaceutical agents such as a chemotherapeutic, steroid, anti-inflammatory compound, or immunosuppressant, examples of which are listed herein.

In certain embodiments, the anti-CD73 antibodies, the inhibitor of A2A and/or A2B adenosine receptor, and/or the inhibitor of PD-1/PD-L1 may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

### Labeled Compounds and Assay Methods

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of an alkyl group of a compound described herein can be optionally substituted with deuterium atoms, such as —CD₃ being substituted for —CH₃).

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of the compounds described herein, are optionally replaced by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro A2A/A2B labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled agent of the disclosure can be used in a screening assay to identify/evaluate agents. For example, a newly synthesized or identified agent (i.e., test agent) which is labeled can be evaluated for its ability to bind an adenosine receptor, CD73, or PD-1/PD-L1 by monitoring its concentration variation when contacting with the adenosine receptor, CD73, or PD-1/PD-L1, respectively, through tracking of the labeling. For example, a test agent (labeled) can be evaluated for its ability to reduce binding of another agent which is known to bind to an adenosine receptor, CD73, or PD-1/PD-L1 (i.e., standard agent). Accordingly, the ability of a test agent to compete with the standard agent for binding to the adenosine receptor, CD73, or PD-1/PD-L1 directly correlates to its binding affinity. Conversely, in some other screening assays, the standard agent is labeled and test agents are unlabeled. Accordingly, the concentration of the labeled standard agent is monitored in order to evaluate the competition between the standard agent and the test agent, and the relative binding affinity of the test agent is thus ascertained.

Accordingly, another aspect of the present disclosure relates to labeled agents (i.e., labeled anti-CD73 antibodies, inhibitors of A2A and/or A2B adenosine receptor, and inhibitors of PD-1/PD-L1) of the disclosure (radio-labeled, fluorescent-labeled, etc.) that would be useful not only in imaging techniques but also in assays, both in vitro and in vivo, for localizing and quantitating CD73, A2A and/or A2B, and/or PD-1/PD-L1 receptors in tissue samples, including human, and for identifying CD73, A2A and/or A2B, and/or PD-1/PD-L1 antagonists by inhibition binding of a labeled compound. Substitution of one or more of the atoms of the compounds of the present disclosure can also be useful in generating differentiated ADME (Adsorption, Distribution, Metabolism and Excretion.) Accordingly, the present disclosure includes adenosine receptor (e.g., A2A and/or A2B) assays that contain such labeled or substituted compounds.

### Kits

The present disclosure also includes pharmaceutical kits useful, for example, in the treatment or prevention of the diseases or disorders described herein, which include one or more containers containing a pharmaceutical composition comprising a therapeutically effective amount of one or more compounds/antibodies of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

Various modifications of the invention, in addition to those described herein, will be apparent to those skilled in the art from the foregoing description. Such modifications are also intended to fall within the scope of the appended claims. Each reference cited in the present disclosure, including all patent, patent applications, and publications, is incorporated herein by reference in its entirety.

### EXAMPLES

The following are examples of the practice of the invention. They are not to be construed as limiting the scope of the invention in any way.

### Example 1. Anti-Tumor Efficacy of ANTIBODY Y in Combination with Retifanlimab and Compound 9

Anti-tumor efficacy of the anti-CD73 antibody, ANTIBODY Y, as a single agent and in combination with A2A/A2B small molecule receptor antagonist, Compound 9 (3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, see Table 1), and/or the anti-PD-1 antibody, retifanlimab, was analyzed in a humanized murine host, carrying the human breast adenocarcinoma tumor, MDA-MB-231, a high expressor of CD73, and established responder to PD-1/PD-L1 blockade. Female human CD34+ reconstituted mice (29 weeks of age; The Jackson Laboratory, Bar Harbor, Me.) were inoculated subcutaneously with 3×10⁶ MDA-MB-231 cells (ATCC #HTB-26), suspended in matrigel (Corning Life Sciences) on their shaved, left flank. On day 7, and every 3 to 4 days subsequently, tumors were measured by Vernier caliper and tumor volume was calculated by the formula, Volume=[L (long dimension)×W² (short dimension)]/2. Based on these measurements, mice were randomized into 8 treatment groups of 10 mice each with an average starting tumor volume of 180 mm³. The studied agents were formulated and administered as follows: ANTIBODY Y was diluted to a final concentration of 1 mg/mL in phosphate-buffered saline and administered by intraperitoneal (i.p.) injection at 10 mL/kg to mice for a dose of 10 mg/kg, every 5 days. Retifanlimab (Macrogenics) was diluted to 1 mg/mL and dosed i.p., every 5 days. For combination treatments, the two antibodies were co-formulated to a concentration of 1 mg/mL, each. The oral vehicle was 5% N,N-dimethylacetamide in 0.5% methyl-cellulose in 50 mM citrate buffer, pH 3.0 (all reagents obtained from Sigma) and was administered by oral gavage (p.o.) twice daily (b.i.d.). Compound 9 (Incyte Corporation) was formulated in the latter vehicle to a concentration of 1 mg/mL, and dosed p.o., b.i.d., daily at 10 mg/mL, for an effective dose of 10 mg/kg. The following treatments and combinations were tested:
1) Vehicle and IgG isotype control;
2) retifanlimab;
3) ANTIBODY Y;
4) Compound 9;
5) retifanlimab+ANTIBODY Y;
6) retifanlimab+Compound 9;
7) ANTIBODY Y+Compound 9; and
8) retifanlimab+ANTIBODY Y+Compound 9.

Dosing began at day 7, and ran for 28 days, through day 35. Animals continued to be tracked individually following the end of dosing to the study's humane endpoint which was achieved when tumor volume was greater than or equal to 10% of the mouse's body weight.

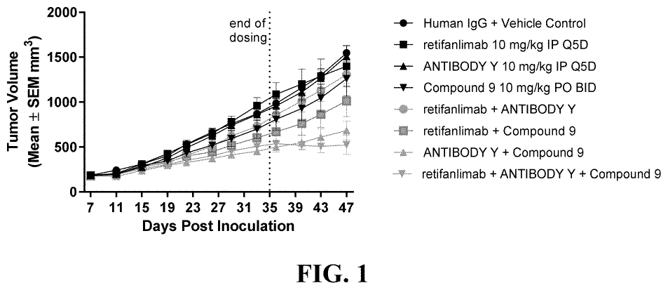

By day 35 when dosing ceased, all the combinations had inhibited tumor growth superior to their component single agents and vehicle. Tumor Growth Inhibition (TGI), defined as (1-treatment group volume)/control group volume)×100 was analyzed for the whole study on day 47, the last day prior to some animals leaving the study at their endpoint. Significance was determined with a non-parametric post test (Kruskal-Wallis) Data is summarized in Table A and FIG. 1.

TABLE A
Day 47 Tumor Growth Inhibition

| Treatment | | |
|---|---|---|
| Vehicle + IgG isotype | Percent TGI | p value |
| retifanlimab | 9.57 | 0.28 |
| ANTIBODY Y | 2.20 | 0.81 |
| Compound 9 | 18.42 | 0.11 |
| retifanlimab + ANTIBODY Y | 15.64 | 0.18 |
| retifanlimab + Compound 9 | 34.58 | 0.02 |
| ANTIBODY Y + Compound 9 | 55.89 | <0.0001 |
| retifanlimab + ANTIBODY Y + | 66.28 | <0.0001 |
| Compound 9 | | |

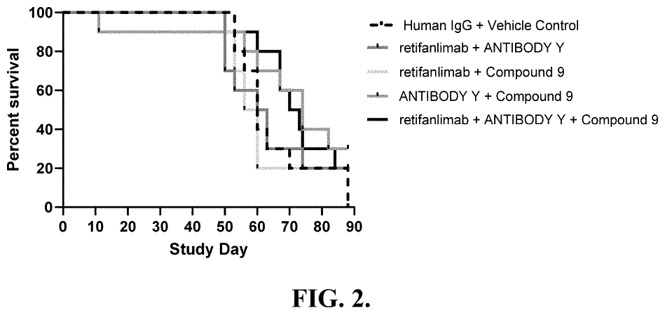

Mice were tracked to their endpoints through day 90, for survival analysis. All combinations with ANTIBODY Y promoted survival greater than that of the vehicle, with median survival of 62 days for the combination with retifanlimab, 74 days with Compound 9, and 72 days for the combination of retifanlimab, Compound 9, and ANTIBODY Y, versus the control group with a median survival of 60 days, as shown in FIG. 2. This data demonstrates that blockade of both CD73 with ANTIBODY Y and A2A/A2B receptors with Compound 9 provides improved disease control and mortality compared to treatment with single agents. Additionally, the best control of tumor growth occurred with the triple combination of ANTIBODY Y, Compound 9, and retifanlimab.

### Example 2. A Phase 1, Open-Label, Multicenter Study of ANTIBODY Y as Monotherapy or in Combination with Immunotherapy in Participants with Advanced Solid Tumors

### I. Objective

This is an open-label, nonrandomized, multicenter, dose escalation, and dose expansion first in human (FIH), Phase 1 study to determine the safety, tolerability, pharmacokinetics (PK), pharmacodynamics, and preliminary efficacy of ANTIBODY Y when given alone or in combination with Compound 9 and/or retifanlimab in participants with specific advanced solid tumors including squamous cell carcinoma of the head and neck (SCCHN) and specified gastrointestinal (GI) malignancies. Participants with CD8 T-cell-positive tumors will be selected as these tumors are more likely to respond to immunotherapy.

### II. Overall Design

Phase 1a will consist of a dose escalation for each treatment group using a hybrid design. This will allow evaluation of the safety and tolerability of the following study treatments in participants with advanced solid tumors (limited to CD8 T-cell-positive advanced SCCHN or specified GI malignancies, defined herein as colorectal cancer (CRC), gastric/gastroesophageal junction (GEJ) cancer, hepatocellular carcinoma (HCC), pancreatic ductal adenocarcinoma (PDAC), or squamous carcinoma of the anal canal (SCAC), after initial dose escalation cohorts):
Treatment Group A (TGA): ANTIBODY Y as monotherapy
Treatment Group B1 (TGB1): ANTIBODY Y in combination with retifanlimab
Treatment Group B2 (TGB2): ANTIBODY Y in combination with Compound 9
Treatment Group C (TGC): ANTIBODY Y in combination with retifanlimab and Compound 9

Following initial dose escalation cohorts in each of the treatment groups, in which participants with advanced solid tumors will be enrolled, the enrollment will be restricted into the subsequent dose escalation cohorts to participants with CD8 T-cell-positive advanced SCCHN or specified GI malignancies (i.e., the same inclusion criteria apply as for Phase 1b) and pre- and on-treatment biopsies will become mandatory. This may occur before opening enrollment into the second dose level or anytime thereafter and will be based on emerging PK data (i.e., saturation of the target mediated drug disposition (TMDD)).

Phase 1b is a dose expansion to better characterize the safety, tolerability, PK, pharmacodynamic effects, and preliminary tumor activity of ANTIBODY Y as monotherapy or in combination with retifanlimab and/or Compound 9 at the recommended dose for expansion (RDE) for the monotherapy and each of the combination therapies in a total of approximately 120 evaluable participants. Participants in Phase 1b will be limited to those with selected CD8 T-cell-positive advanced or metastatic SCCHN or specified GI malignancies (defined herein as colorectal cancer (CRC), gastric/gastroesophageal junction (GEJ) cancer, hepatocellular carcinoma (HCC), pancreatic ductal adenocarcinoma (PDAC), or squamous carcinoma of the anal canal (SCAC)).
TGA (ANTIBODY Y monotherapy)
   SCCHN: 10 participants
   Specified GI malignancies: 10 participants
TGB1 (ANTIBODY Y+retifanlimab)
   SCCHN: 10 participants
   Specified GI malignancies: 10 participants
TGB2 (ANTIBODY Y+Compound 9)
   SCCHN: 20 participants
   Specified GI malignancies: 20 participants
TGC (ANTIBODY Y+retifanlimab+Compound 9)
   SCCHN: 20 participants
   Specified GI malignancies: 20 participants

The study will include a 28-day screening period to determine eligibility, a treatment period of up to 2 years, an end of treatment (EOT) visit, and 30-day and 90-day safety follow-up visits. Participants who discontinue study treatment for a reason other than disease progression will continue to be assessed for their disease status during the follow-up phase and should continue to have tumor assessments every 8 weeks for the first 12 months and then every 12 weeks thereafter until a new anticancer therapy is started, disease progression, death, withdrawal of consent, or the end of the study, whichever occurs first.

Tumor assessments will be performed at baseline and subsequently every 8 weeks for the first year of treatment and every 12 weeks thereafter by site investigator review according to Response Evaluation Criteria in Solid Tumors (RECIST) v1.1. Guidance per immune Response Evaluation Criteria in Solid Tumors (iRECIST) may be used for decisions to discontinue study treatment due to radiologic progressive disease (PD).

Safety will be evaluated from the time the participant signs informed consent until the 90-day safety follow-up visit. Safety data will also be reviewed periodically by a Safety Review Committee.

Mandatory pretreatment and on-treatment biopsies will be collected from all participants with the exception of early dose levels in each of the treatment groups in the dose-escalation Phase 1a as described above. If an adequate on-treatment tumor tissue cannot be obtained, the participant will be allowed to continue in the study.
In Phase 1a, up to 6 additional participants may be enrolled in a dose level in the event that insufficient evaluable paired biopsy specimens have been obtained.
In Phase 1b, the aim is to obtain at least 10 evaluable paired biopsy specimens within each cohort. Up to 6 additional participants may be enrolled in a cohort (i.e., SCCHN or specified GI malignancies) in case 10 evaluable paired specimens have not been obtained following initial enrollment of participants.

CD8 T-cell-positive tumors are required for entry into the study for all participants (with the exception of those in the early dose levels in each of the treatment groups in the dose-escalation Phase 1a). The mandatory pretreatment biopsies collected from all participants will be analyzed for the presence of CD8+ T-cell lymphocytes as part of prescreening. Prescreening allows preselection of participants with CD8 T-cell-positive tumors to be performed outside of the 28-day screening period and before signing the main informed consent form (ICF) for the study. Participants will be required to sign a specific prescreening consent form; however, no other Protocol assessments will be performed under the prescreening consent.

Participants in TGA of Phase 1a and Phase 1b with available archival tissue may submit the archival tissue for this prescreening analysis; however, eligible participants will be required to undergo a fresh biopsy during the screening period for biomarker analyses. These fresh biopsies during screening are required to obtain frozen tissue samples necessary for evaluation of CD73 enzymatic activity.

The paired tumor biopsies collected in the study will be used to demonstrate pharmacodynamic activity of ANTIBODY Y (on fresh paired biopsy specimens), evaluate changes in the tumor and tumor microenvironment (TME), identify potential biomarkers, and develop and evaluate an adenosine-regulated gene expression signature.

III. Phase 1a—Dose Escalation

An open-label hybrid design will be used to assess the safety and tolerability and to identify the RDE for TGA and the combination treatment groups TGB1, TGB2 and TGC in participants with advanced solid tumors (limited to CD8 T-cell positive advanced SCCHN or specified GI malignancies (CRC, GEJ cancer, HCC, PDAC, or SCAC) after initial dose escalation cohorts). Details for each of the treatment groups are found in the relevant subsequent subsections.

Dose escalation will begin with TGA. The decision to open enrollment to dose escalation cohorts for TGB1, TGB2, and TGC will be based on the observed safety, tolerability, clinical activity, PK, and pharmacodynamics of ANTIBODY Y.

### Hybrid Statistical Design to Guide Dose Escalation

The hybrid design is a hybrid of the modified toxicity probability interval design and a dose-toxicity model, and it has 3 steps.

Step 1. A modified toxicity probability interval (mTPI) design (see e.g., Ji et al Clin. Trials. 2010; 7:653-663) with a target dose-limiting toxicity (DLT) rate p_{T} of 28% is first modified to control the overdosing toxicity using the posterior probability of DLT rate in the overdosing interval (p_{T}+ε₂,1) to be less than 0.8. With this rule, if 3 DLTs are observed out of 6 participants which is a DLT rate of about 50%, then the modified mTPI will guarantee a dose de-escalation instead of staying at the current dose level when the observed toxicity rate is high. Table B shows the dose-escalation rules based on the number of DLT observed in a dose level cohort, where E=escalate to the next higher dose; D=de-escalate to the next lower dose; DU=the current dose is unacceptably toxic; S=stay at the current dose. Target toxicity rate p_{T}: 28%. Flat noninformative prior Beta(1,1) is used as a prior and ε₁=ε₂=0.05 (see e.g., Ji et al, Clin. Trials 2010, 7:653-663; and Ji et al, J. Clin. Oncol. 2013, 31:1785-1791). Posterior toxicity probability cut: 0.8.

TABLE B

| Participants | | | | | | | |
|---|---|---|---|---|---|---|---|
| With at | Number of Participants Evaluable for DLT | | | | | | |
| Least 1 DLT | 3 | 4 | 5 | 6 | 7 | 8 | 9 |
| 0 | E | E | E | E | E | E | E |
| 1 | S | S | S | S | E | E | E |
| 2 | D | S | S | S | S | S | S |
| 3 | DU | DU | D | D | S | S | S |
| 4 | | DU | DU | DU | DU | D | S |
| 5 | | | DU | DU | DU | DU | DU |
| 6 | | | | DU | DU | DU | DU |
| 7 | | | | | DU | DU | DU |
| 8 | | | | | | DU | DU |
| 9 | | | | | | | DU |

Step 2. The second step of the hybrid design is to use a dose-toxicity model by pooling all observed safety information from all previous doses to estimate the DLT rate for the current dose level and predict the DLT rate for the next dose level in the provisional dose list. The estimated DLT rate at the current dose level is used together with the decision rules from the modified mTPI in Table B to make a decision jointly about dose escalation. If the dose-toxicity model is not feasible (e.g., no DLT observed in any tested doses) then no action is needed at this step.

Step 3. If the decision in Table B is to have a dose-escalation (E) to the next dose level in the provisional dose list, then the predicted DLT rate using the dose-toxicity model from Step 2 is used to judge whether the next dose level is feasible or not by checking whether the predicted DLT rate at the next dose level is over the prespecified targeted DLT rate. If the predicted DLT rate is over the targeted DLT rate, then the next dose level in the provisional dose list cannot be used. Instead, an intermediate dose from the dose-toxicity model will be calibrated so that the DLT rate is below the targeted DLT rate. If the decision in Table B is to have a dose de-escalation (D) to a lower dose level in the provisional dose list, an intermediate dose from the earlier used dose-toxicity model will be calibrated so that the DLT rate is below the targeted DLT rate. Note that choosing the intermediate dose level will take into consideration what is clinically and operationally feasible (eg, based on exposure interparticipant variability). If the decision in Table B is to stay (S) at the current dose, then the estimated DLT rate at the current dose using the dose-toxicity model from Step 2 is used to make a decision. If the estimated DLT at the current dose is over the prespecified targeted DLT rate, then the decision is to dose de-escalate (D); otherwise, it is a stay (S).

A minimum of 3 evaluable participants are required at each dose level. However, depending on the accrual rate, 3, 4, 5, or 6 participants may be enrolled. In each treatment group, approximately 30 evaluable participants may be treated in the dose-escalation stages, and the dose-escalation procedure may be stopped if the number of evaluable participants treated at any dose level is ≥9. If emerging data supports de-escalation of an unacceptable dose (D or DU) at the lowest dose level, the study will evaluate the data to determine whether a lower dose (or alternative schedule) should be considered.

When adding participants to a dose level in response to a “Stay (S)” decision, the number of additional participants to be enrolled is capped to minimize the exposure to a dose that may have unacceptable toxicity (denoted as “Dose Unacceptable (DU)” in Table B). Secondly, to determine how many more participants can be enrolled at the dose level, one can count steps in a diagonal direction (down and to the right) from the current cell to the first cell marked DU. For example, if 1 of 3 participants have experienced a DLT at a given dose level, no more than 3 additional participants should be enrolled at this dose level until additional DLT data are available. This is because that dose level would be considered unacceptably toxic if all 3 of the additional participants experience a DLT (i.e., 4 of 6 participants with a DLT in Table B).

If no DLT is observed in all proposed doses and there is no clear efficacy related signal in the highest dose level, then the dose-escalation procedure may be continued with additional participants enrolled at a higher dose level.

At the end of the dose-escalation procedure, the DLT rates at all tested dose levels will be estimated based on the aforementioned dose-toxicity model if it is feasible or the pool-adjacent-violators algorithm if the parametric dose-toxicity model is not feasible. The dose with an estimated DLT rate closest to 28% will be treated as a MTD. However, the totality of the available data such as the emerging safety, PK, progressive disease (PD), and other biomarker information will be considered before deciding on the dose(s) to carry forward to Phase 1b.

### Treatment Group A—ANTIBODY Y Monotherapy

Enrollment for dose escalation will start with ANTIBODY Y monotherapy at a dose of 70 mg every two weeks (Q2W) administered intravenous (IV) on Day 1 and 15 of each 28-day cycle. The DLT evaluation period is 28 days long, and safety and tolerability will be reviewed when evaluable participants in a dose level cohort pass the 28-day DLT period before opening the next dose level cohort. The proposed dosage (70 mg Q2W) attempts to minimize the exposure of late-stage cancer patients to subtherapeutic dose levels of ANTIBODY Y while balancing the safety risk associated with the nonclinical pharmacologic and toxicological profiles. This dose was determined from the weight-of-evidence (WOE) of all nonclinical data and is considered to provide an acceptable risk-benefit profile.

Planned dose levels of ANTIBODY Y to be explored in this study may include 70 mg, 250 mg, 750 mg, and 1500 mg, but doses will be selected based on emerging data. Doses above 250 mg will not increase by more than three-fold. Intermediate dose levels may be explored if supported by safety, PK, or pharmacodynamic data.

Participants must have received 2 doses of ANTIBODY Y at the level assigned for Q2W dosing or 1 dose of ANTIBODY Y at the level assigned for Q4W dosing during the 28-day DLT observation period or have had a DLT to be evaluable for dose tolerability. Participants who are considered not evaluable for reasons other than toxicity may be replaced. In addition, participants with late-onset safety events meeting the definition of a DLT or those who had intolerable, lower-grade persistent toxicity determined to be attributable to study drug (e.g., Grade 2 peripheral neuropathy) will be considered in the selection of the RDE.

Dose interruptions and/or modifications may be implemented based on toxicity. Dose modifications should not be made during the DLT observation period without discussion with the medical monitor. If a dose level is deemed unacceptably toxic, all participants enrolled into that dose level may decrease their dose to the last dose level determined to be tolerable.

Up to a total of 6 additional participants may be enrolled at any tolerable dose level to further investigate safety, PK, and/or pharmacodynamic biomarkers. Participants enrolled for the purpose of assessing pharmacodynamic biomarkers will also be required to provide pre- and on-treatment tumor biopsies and to have SCCHN or a specified GI malignancy.

The administration schedule of ANTIBODY Y may be changed from Q2W to every four weeks (Q4W) based on emerging PK and pharmacodynamic data. Q4W dosing is more convenient for participants and aligns well with Q4W dosing of retifanlimab for participants receiving a combination therapy including retifanlimab (TGB1 and TGC). At each dose level, 1 participant will be treated first with a waiting period of ≥24 hours later before treatment start of the remaining participants.

### Treatment Group B1—ANTIBODY Y+Retifanlimab

Retifanlimab will be administered at 500 mg IV Q4W in all dose levels. The selection of the retifanlimab 500 mg Q4W dose was based on modeling of clinical PK data from the first-in-human monotherapy study (see e.g., clinicaltrials.gov, NCT03059823), that evaluated both weight-based dosing at doses ranging from 1 to 10 mg/kg Q2W or Q4W and flat dosing at doses of 375 mg Q3W, 500 mg Q4W, and 750 mg Q4W in 219 participants.

Enrollment in TGB1 may start once at least 2 dose levels in TGA have been declared tolerable or the RDE has been selected. In addition, available PK and pharmacodynamic data from TGA of the study will be used to help guide initiation of TGB1. The decision to open TGB1 will be made by agreement between the medical monitor and the study investigators. To ensure safety of the combination treatment, the ANTIBODY Y starting dose in TGB1 will be 1 dose level below or at least 50% less (whichever is higher) than the highest tested tolerated dose of ANTIBODY Y in TGA at the time of opening TGB1.

ANTIBODY Y may be administered Q2W or Q4W in combination with retifanlimab. The dose escalation criteria for ANTIBODY Y in TGB1 will be the same as that used for the ANTIBODY Y monotherapy dose escalation in TGA; that is, doses above 250 mg will not increase by more than 3-fold, and intermediate dose levels (from planned dose levels) may be explored. At each dose level, 1 participant will be treated first with a waiting period of ≥24 hours before treatment start of the remaining participants.

TGB1 dose escalation will follow the same hybrid design as outlined for TGA. Participants must have received 2 doses of ANTIBODY Y at the level assigned for Q2W dosing or 1 dose of ANTIBODY Y at the level assigned for Q4W dosing and 1 dose of retifanlimab during the 28-day DLT observation period or have had a DLT to be evaluable for dose tolerability. In addition, participants with late-onset safety events meeting the definition of a DLT or those who had intolerable, lower-grade persistent toxicity determined to be attributable to study drug (e.g., Grade 2 peripheral neuropathy) will be considered in the selection of the RDE.

Dose interruptions and/or modifications for ANTIBODY Y may be implemented based on toxicity. Dose modifications should not be made during the DLT observation period without discussion with the medical monitor. If a dose level is deemed unacceptably toxic, all participants enrolled into that dose level may decrease their dose to the last dose level determined to be tolerable.

At the discretion of the sponsor, up to a total of 6 additional participants may be enrolled at any tolerable dose level to further investigate safety, PK and/or pharmacodynamic biomarkers. Participants enrolled for the purpose of assessing pharmacodynamic biomarkers will also be required to provide pre- and on-treatment tumor biopsies and to have SCCHN or a specified GI malignancy.

### Treatment Group B2—ANTIBODY Y+Compound 9

Enrollment in TGB2 may start once at least 2 dose levels in TGA have been declared tolerable or the RDE has been selected. In addition, available PK and pharmacodynamic data from TGA of the study will be used to help guide initiation of TGB2. The final decision to open TGB2 will be made by agreement between the medical monitor and the study investigators. To ensure safety of the combination treatment, the following will apply:
ANTIBODY Y starting dose in TGB2 will be 1 dose level below or at least 50% less (whichever is higher) than the highest tested tolerated dose of ANTIBODY Y in TGA at the time of opening TGB2.
Compound 9 starting dose in TGB2 will be 1 dose level below or at least 50% less (whichever is higher) than the highest tested tolerated dose or RDE of Compound 9 as monotherapy.

ANTIBODY Y may be administered Q2W or Q4W in combination with Compound 9. The dose escalation criteria for ANTIBODY Y in TGB2 will be the same as that used for the ANTIBODY Y monotherapy dose escalation in TGA; that is, ANTIBODY Y doses above 250 mg will not increase by more than 3-fold, and intermediate dose levels (from planned dose levels) may be explored. Compound 9 may be administered QD or twice daily (BID) in combination with ANTIBODY Y. Dose increases of Compound 9 will never exceed 100% (i.e., 2-fold increase). Following observation of ≥Grade 2 toxicities that have a reasonable possibility of being related to study treatment are observed in at least 2 participants at the previous Compound 9 dose level, subsequent increases in Compound 9 will be limited to no more than 50% in successive Compound 9 dose levels. At each dose level, 1 participant will be treated first with a waiting period of ≥24 hours before treatment start of the remaining participants.

In TGB2, parallel dose levels may be opened where ANTIBODY Y is escalated in one dose level cohort and Compound 9 is escalated in the other dose level cohort. Only 1 study drug will be escalated in a dose level. Therefore, TGB2 dose escalation for either ANTIBODY Y or Compound 9 will follow the same hybrid design as outlined TGA.

Participants must have received 2 doses of ANTIBODY Y at the level assigned for Q2W dosing or 1 dose of ANTIBODY Y at the level assigned for Q4W dosing and at least 75% of doses of Compound 9 (i.e., 21 of 28 doses for QD dosing [42 of 56 doses in case of BID dosing]) at the level assigned during the 28-day DLT observation period or have had a DLT to be evaluable for dose tolerability. In addition, participants with late-onset safety events meeting the definition of a DLT or those who had intolerable, lower-grade persistent toxicity determined to be attributable to study drug (e.g., Grade 2 peripheral neuropathy) will be considered in the selection of the RDE.

Dose interruptions and/or modifications may be implemented based on toxicity. Dose modifications should not be made during the DLT observation period without discussion with the medical monitor. If a dose level is deemed unacceptably toxic, all participants enrolled into that dose level may decrease their dose to the last dose level determined to be tolerable.

At the discretion of the sponsor, up to a total of 6 additional participants may be enrolled at any tolerable dose level to further investigate safety, PK, and/or pharmacodynamic biomarkers. Participants enrolled for the purpose of assessing pharmacodynamic biomarkers will also be required to provide pre- and on-treatment tumor biopsies and to have SCCHN or a specified GI malignancy.

### Treatment Group C—ANTIBODY Y+Retifanlimab+Compound 9

Initiation of enrollment for the triplet combination treatment of ANTIBODY Y+Compound 9+retifanlimab in the dose-escalation portion of the study may occur under one of the following conditions:
After at least 2 dose levels of one of TGB1 or TGB2 have been declared as tolerable or the RDE has been selected; or
After at least 2 dose levels of ANTIBODY Y in TGA in this study and at least 2 dose levels of Compound 9+retifanlimab have been declared as tolerable or the RDE has been selected.

Available PK and pharmacodynamic data from prior cohorts and treatment groups will be used to help guide initiation of TGC. To ensure safety of the triplet combination, the following will apply:
The starting dose of ANTIBODY Y will be defined as 1 dose level below or at least 50% less (whichever is higher) than the highest tested tolerated dose of ANTIBODY Y when given as monotherapy on in combination with retifanlimab or Compound 9 (if the tolerated dose of ANTIBODY Y is different in TGB1 and TGB2, the starting dose for the triplet will be 1 dose level below or at least 50% less than the lower of the tolerated doses from the doublet combinations).
The starting dose of Compound 9 will be defined as 1 dose level below or at least 50% less (whichever is higher) than the highest tolerated dose of Compound 9 when given in combination with ANTIBODY Y in TGB2 of this study or the highest tolerated dose of Compound 9 with retifanlimab.

Retifanlimab will be administered at 500 mg IV Q4W in all dose levels. ANTIBODY Y may be administered Q2W or Q4W in combination with Compound 9 and retifanlimab. The dose of ANTIBODY Y and Compound 9 may be escalated in this treatment group. Only 1 study drug will be escalated within a cohort, although parallel cohorts may be enrolled. The dose escalation criteria for ANTIBODY Y in TGC will be the same as that used for the ANTIBODY Y monotherapy dose escalation in TGA; that is, ANTIBODY Y doses above 250 mg will not increase by more than 3-fold, and intermediate dose levels (from planned dose levels) may be explored. Compound 9 may be administered QD or BID in combination with ANTIBODY Y and retifanlimab. The dose escalation criteria for Compound 9 in TGC will be the same as that described for Compound 9 in TGB2; that is, dose increases in successive Compound 9 dose levels will be up to 2-fold until treatment-related ≥Grade 2 toxicities are observed in at least 2 participants at the previous Compound 9 dose level. Following observation of such a toxicity, subsequent dose increases will be limited to no more than 50% at the successive Compound 9 dose level. At each dose level, 1 participant will be treated first with a waiting period of ≥24 hours before treatment start of the remaining participants.

Parallel dose levels may be opened where ANTIBODY Y is escalated in one dose level cohort and Compound 9 is escalated in the other dose level cohort as outlined for TGB2. As with TGB2, only 1 of the study drugs ANTIBODY Y or Compound 9 will be escalated in a dose level. Therefore, TGC dose escalation for either ANTIBODY Y or Compound 9 will follow the same hybrid design as outlined for TGA.

Participants must have received (a) 2 doses of ANTIBODY Y at the level assigned for Q2W dosing or 1 dose of ANTIBODY Y at the level assigned for Q4W dosing; (b) 1 dose of retifanlimab; and (c) at least 75% of doses of Compound 9 (i.e., 21 of 28 doses for QD dosing [42 of 56 doses in case of BID dosing]) at the level assigned during the 28-day DLT observation period or have had a DLT to be evaluable for dose tolerability. In addition, participants with late-onset safety events meeting the definition of a DLT or those who had intolerable, lower-grade persistent toxicity determined to be attributable to study drug (eg, Grade 2 peripheral neuropathy) will be considered in the selection of the RDE.

Dose interruptions and/or modifications may be implemented based on toxicity. Dose modifications should not be made during the DLT observation period without discussion with the medical monitor. If a dose level is deemed unacceptably toxic, all participants enrolled into that dose level may decrease their dose to the last dose level determined to be tolerable.

Up to a total of 6 additional participants may be enrolled at any tolerable dose level to further investigate safety, PK, and/or pharmacodynamic biomarkers. Participants enrolled for the purpose of assessing pharmacodynamic biomarkers will also be required to provide pre- and on-treatment tumor biopsies and to have SCCHN or a specified GI malignancy.

### Definition of the Recommended Dose for Expansion (RDE)

The RDE for ANTIBODY Y as monotherapy and each of the combination treatments (TGB1, TGB2, and TGC) will be determined by evaluation of all available data, including safety as well as PK and pharmacodynamic data, from the dose-escalation portion of the study within each dose level cohort for further investigation in the dose expansion part (Phase 1b) of the study. The individual drug dose level of ANTIBODY Y and Compound 9 in the combination treatment groups should not exceed, but may be equal to the RDE for each individual drug as monotherapy.

IV. Phase 1b—Dose Expansion

An expansion is included to further explore safety, tolerability, pharmacokinetics, pharmacodynamic effects, and preliminary antitumor activity of TGA or in combination groups TGB1, TGB2, and TGC at the RDE for the monotherapy and each of the combination therapies identified in Phase 1a.

Phase 1b will focus primarily on participants with CD8 T-cell positive SCCHN and specified GI tumors: CRC, GEJ cancer, HCC, PDAC, or SCAC in order to obtain additional data on the study treatments at the RDE in these selected tumor types. There is a high unmet medical need for participants in these population in later lines of therapy when SoC options have been exhausted.

After enrollment in the dose-expansion cohorts has begun, further enrollment of participants within a specific cohort (i.e., SCCHN or specified GI malignancies) in one of the treatment groups will be suspended if (1) >1 participant in the first 5 participants enrolled in that cohort have an adverse event (AE)≥Grade 3 that is attributable to the study treatment, or (2) >40% of 5 or more participants enrolled in that cohort have an AE ≥Grade 3 that is attributable to the study treatment.

Enrollment of participants within a specific cohort in one of the treatment groups will be suspended until the sponsor, investigators, and regulatory authorities (if applicable) have determined the appropriate course of action.

### Treatment Group A—ANTIBODY Y Monotherapy

TGA will include up to 20 participants across 2 tumor-specific cohorts:
SCCHN: 10 participants
Specified GI malignancies: 10 participants
   Additional (tumor-specific) cohorts may be added, by protocol amendment, based on emerging data.

### Treatment Group B1—ANTIBODY Y+Retifanlimab

TGB1 will include up to 20 participants across 2 tumor-specific cohorts:
SCCHN: 10 participants
Specified GI malignancies: 10 participants
   Additional (tumor-specific) cohorts may be added, by protocol amendment, based on emerging data.

### Treatment Group B2—ANTIBODY Y+Compound 9

TGB2 will include up to 40 participants across 2 tumor-specific cohorts:
SCCHN: 20 participants
Specified GI malignancies: 20 participants
   Additional (tumor-specific) cohorts may be added, by protocol amendment, based on emerging data.

### Treatment Group C—ANTIBODY Y+Retifanlimab+Compound 9

TGC will include up to 40 participants across 2 tumor-specific cohorts:
SCCHN: 20 participants
Specified GI malignancies: 20 participants
   Additional (tumor-specific) cohorts may be added, by protocol amendment, based on emerging data.

### Add-On Therapy

Participants in the dose escalation (Phase 1a) and dose expansion (Phase 1b) will have the potential to receive add-on treatment with retifanlimab or Compound 9 as follows:
Participants enrolled into TGA may receive add-on treatment with either Compound 9 or retifanlimab
Participants enrolled into TGB1 may receive add-on treatment with Compound 9
Participants enrolled into TGB2 may receive add-on treatment with retifanlimab

Participants will be allowed to receive the add-on therapy after at least 2 cycles of study treatment in the respective treatment group and in the absence of an objective response (i.e., partial response (PR) or complete response (CR)) or clinical benefit (i.e., stable disease (SD) (e.g., tumor shrinkage not meeting the criteria for objective response, and no worsening of clinical symptoms)), or following disease progression.

In Phase 1a, add-on therapy in TGA may only be given if 2 dose levels in TGA have been declared tolerable and the dose escalation for the combination at the corresponding dose of ANTIBODY Y has been declared tolerable (e.g., a participant receiving ANTIBODY Y 250 mg Q2W can receive ANTIBODY Y 250 mg Q2W+retifanlimab only after the ANTIBODY Y 250 mg Q2W dose level in TGB1 has been declared tolerable). Similarly, participants in TGB1 or TGB2 in Phase 1a may also receive add-on treatment with the third agent to receive the triplet therapy following the same instructions as described above for TGA.

Participants in both Phase 1a and 1b who begin on monotherapy will be allowed to receive only a single add-on treatment (i.e., they cannot receive a second add-on therapy to receive the triplet therapy). Participants will be analyzed for safety and efficacy within the originally assigned treatment group until initiation of the add-on therapy. After start of the add-on therapy, they will be analyzed as a separate group.

### V. Study Treatments

TABLE C-1

| | |
|---|---|
| Study | ANTIBODY Y |
| treatment | |
| name: | |
| Mechanism | CD73 inhibitor |
| of action: | |
| Dosage | Solution for infusion |
| formulation: | |
| Unit dose | 50 mg/mL |
| strength(s)/ | |
| dosage | |
| level(s): | |
| Administration | Administered IV over 30 minutes (−5/+15 min) using a |
| instructions: | filter. |
| | On clinic visits when both ANTIBODY Y and |
| | retifanlimab are to be administered, ANTIBODY Y |
| | should be infused first, followed by a 30-minute |
| | wait before beginning the retifanlimab infusion. |
| Administration | |
| instructions | |
| (continued): | |
| Packaging and | ANTIBODY Y will be provided in a glass vial |
| labeling: | (50 mg/mL) for single use. Each vial will be labeled |
| | as required per country requirement. |
| Storage: | Must be refrigerated upright and protected from light. |
| | Store at 2° C.-8° C. (36° F.-46° F.). |

TABLE C-2

| | |
|---|---|
| Study | Retifanlimab |
| treatment | |
| name: | |
| Mechanism | PD-1 inhibitor |
| of action: | |
| Dosage | Liquid formulation |
| formulation: | |
| Unit dose | 25 mg/mL |
| strength(s)/ | |
| dosage | |
| level(s): | |
| Administration | Administered IV over 30 minutes (−5/+15 min) using a |
| instructions: | filter |
| Administration | |
| instructions | |
| (continued): | |
| Packaging and | Retifanlimab will be provided in a glass vial |
| labeling: | (25 mg/mL) for single use. |
| | Each vial will be labeled as required per country |
| | requirement. |
| Storage: | Must be refrigerated upright and protected from light. |
| | Store at 2° C.-8° C. (36° F.-46° F.). |

TABLE C-3

| | |
|---|---|
| Study | Compound 9 |
| treatment | |
| name: | |
| Mechanism | Adenosine A2A/A2B receptor inhibitor |
| of action: | |
| Dosage | Immediate release tablets |
| formulation: | |
| Unit dose | 10 mg or 40 mg |
| strength(s)/ | |
| dosage | |
| level(s): | |
| Administration | Administered orally (PO) in 10 mg and/or 40 mg tablet QD in the |
| instructions: | morning on each day of each 28-day cycle. In case of BID dosing |
| | evaluation, BID rning and evening, approximately 12 hours apart, |
| | on each day of the 28-day cycle. |
| | Compound 9 should be administered with water and may be taken |
| | with or without food, except on days when predose PK samples for |
| | Compound 9 (TGB2 and TGC) are collected. |
| | If a QD dose is missed by more than 12 hours, then that dose should |
| | be skipped, and the next scheduled dose should be taken at the usual |
| | time. |
| | (in case of BID dosing evaluation, if the morning or evening dose is |
| | missed by more than 4 hours, then that dose should be skipped, and |
| | the next scheduled dose should be taken at the usual time) |
| | On days when PK samples are collected for Compound 9 (TGB2 |
| | and TGC), participants should fast for at least 2 hours before and |
| | remain fasting from food for at least 1 hour postdose, after which a |
| | meal or snack may be consumed. |
| Administration | On days when Compound 9 samples are collected for PK (TGB2 |
| instructions | and TGC), anti-drug antibody (ADA), whole blood for receptor |
| (continued): | occupancy assessment and/or plasma for correlative studies, |
| | Compound 9 should be taken in the clinic after the respective |
| | predose blood/plasma samples have been collected. |
| | On days when ANTIBODY Y and retifanlimab (if applicable) will |
| | be administered, Compound 9 should be taken before other study |
| | treatments |
| Packaging and | Compound 9 tablets will be provided in bottles. |
| labeling: | Each bottle will be labeled as required per country requirement. |
| Storage: | Store at room temperature (15° C.-30° C. [59° F.-86° F.]). |

### VL. Efficacy Assessment

Objective assessment of disease status is required, using the evaluations by RECIST v1.1 (e.g., Eisenhauer et al. Eur. J. Cancer, 2009, 45:228-247). Efficacy baseline assessments will be performed at screening, and further efficacy assessments will be performed throughout the study.

### Tumor Imaging by RECIST v1.1

The same imaging technique should be used for a participant throughout the study. The baseline scan must be a contrast computed tomography (CT) or magnetic resonance imaging (MRI), except in circumstances where there is a contrast allergy or with medical monitor approval. When the CT component of a positron emission tomography/CT scan uses higher energy and thinner slices, it may be acceptable with medical monitor approval. Images of the chest, abdomen, and pelvis are required for all participants. Additional imaging of anatomical sites (e.g., head, neck, brain), should be performed as applicable for the malignancy under study.

A CT or MRI scan of the brain will be performed at screening if there are signs or symptoms suggesting that the participant has disease involvement in the CNS.

### Baseline Assessment During Screening

Initial tumor imaging must be performed within 28 days before the first dose of study treatment. The site study team must review prestudy reports and images to confirm that the participant has measurable disease per RECIST v1.1. Tumor lesions that are located in a previously irradiated area or in an area subjected to other locoregional therapy should not be selected as target lesions. Participants with a single target lesion that has been previously irradiated or subjected to other local-regional therapy may be enrolled if the target lesion is considered measureable per RECIST v1.1 and has demonstrated at least a 10 mm increase in the shortest diameter of the lesion. Additionally, it is recommended that tumor lesions selected for biopsy not be selected as target lesions.

Scans performed as part of routine clinical management are acceptable for use as the screening scan if they are of diagnostic quality and performed within 28 days before the first dose of study treatment.

### Assessment of Disease Response During Treatment

The first imaging assessment should be performed 8 weeks after the first dose of study treatment and then every 8 weeks (±7 days) for the first 12 months. After 12 months of study treatment, imaging frequency may be reduced to every 12 weeks (±14 days). Imaging assessments may be performed more frequently if clinically indicated. Imaging should follow calendar days and should not be delayed for delays in cycle starts. Response (CR or PR) should be confirmed by imaging at least 4 weeks after initial documentation of response.

Disease progression should be confirmed at least 4 weeks but no more than 8 weeks after the first scan indicating disease progression in clinically stable participants as per iRECIST guidelines Participants who have unconfirmed disease progression may continue on treatment until progression is confirmed.

### Assessment of Disease Response After Treatment

If the participant discontinues study treatment for reasons other than disease progression, imaging assessments should continue at the Protocol-specified interval of approximately every 8 weeks (±7 days) for the first 12 months and then every 12 weeks (±14 days) thereafter until documented disease progression, the start of new anticancer treatment, withdrawal of consent, death, or the end of the study, whichever occurs first (for up to a maximum of 2 years from end of treatment (EOT).

### VII. Pharmacokinetic Assessments

### Blood Sample Collection

Blood will be collected for the determination of ANTIBODY Y serum concentrations, retifanlimab serum concentrations, and Compound 9 plasma concentrations.

All samples will be analyzed using validated methods. Blood samples will be collected from the arm contralateral to the site of IV infusion. If an indwelling catheter is used, the fluid in the catheter will be removed and discarded before the collection of blood sample for PK assessment.

Timing of blood collection for PK assessments is outlined in Table D for TGA and TGB1 and Table E for TGB2 and TGC. After the preinfusion/predose PK sample is drawn, participants will begin the study treatment. Predose is defined as within 30 minutes before administration of study treatment.

For participants enrolled into TGB2 or TGC, on PK assessment visits during which predose Compound 9 samples are collected (as per Table E), participants must refrain from taking Compound 9 before arriving for the visit and should not have consumed any food within 2 hours before arriving at the site. Following predose PK sampling and subsequent Compound 9 administration, food should be withheld until 1 hour after Compound 9 administration.

The exact date and time of the PK blood draws will be recorded along with the date and time of the last dose of Compound 9 study drug preceding the blood draw (if applicable) and the time of the most recent meal. Participants in TGB2 and TGC will be instructed, and reminded, to hold the dose of Compound 9 and consumption of food on the day of a visit during which predose Compound 9 PK samples will be collected. Participants will be instructed, and reminded, to provide the date and time of their prior dose of Compound 9 study drug and date and time of the most recent meal or snack consumed.

Adjustments to the timing of blood sampling may be made based on emerging PK data. Additional PK samples may be collected and evaluated during the study if warranted (e.g., in case a participant receives restricted medication or in case of any safety concerns or overdose arising during the study).

TABLE D

| Study Visit | Timing of Sample |
|---|---|
| Cycle 1 Day 1 | Preinfusion |
| | Immediately after ANTIBODY Y infusion |
| | (≤10 min)^{a} |
| | Immediately after retifanlimab infusion (≤10 min), if |
| | applicable |
| | 6 hours postinfusion (±1 h) of ANTIBODY Y |
| Cycle 1 Day 2 | Anytime |
| Cycle 1 Day 8 | Anytime |
| Cycle 1 Day 15 if on a Q2W | Preinfusion |
| schedule of ANTIBODY Y | Immediately after ANTIBODY Y infusion |
| | (≤10 min) |
| Cycle 1 Day 15 if on a Q4W | Anytime |
| schedule of ANTIBODY Y | |
| Cycle 1 Day 22 | Anytime |
| Cycle 2 Day 1 | Preinfusion |
| | Immediately after ANTIBODY Y infusion |
| | (≤10 min)^{a} |
| | Immediately after retifanlimab infusion (≤10 min), if |
| | applicable |
| Cycle 2 Day 8 | Anytime |
| Day 1 of every other cycle | Preinfusion |
| starting at Cycle 4 Day 1 (ie, | |
| C4D1, C6D1, C8D1, etc) | |
| 30-day safety follow-up visit | Anytime |

^{a}Sample is to be collected before starting retifanlimab infusion if applicable

TABLE E

| Study Visit | Timing of Sample |
|---|---|
| Cycle 1 Day 1 | Preinfusion of ANTIBODY Y and predose of |
| | Compound 9 |
| | Immediately after ANTIBODY Y infusion |
| | (≤10 min)^{a} |
| | Immediately after retifanlimab infusion (≤10 min), if |
| | applicable |
| | 1 hour post- Compound 9 dosing (±15 min), only |
| | Compound 9 will be measured |
| | 2 hours post- Compound 9 dosing (±15 min), only |
| | Compound 9 will be measured |
| | 4 hours post- Compound 9 dosing (±30 min), only |
| | Compound 9 will be measured |
| | 6 hours post-infusion (±1 h) of ANTIBODY Y |
| Cycle 1 Day 2 | Predose for Compound 9 |
| Cycle 1 Day 8 | Predose for Compound 9 |
| Cycle 1 Day 15 if on a Q2W | Preinfusion and predose of Compound 9 |
| schedule of ANTIBODY Y | Immediately after ANTIBODY Y infusion |
| | (≤10 min) |
| Cycle 1 Day 15 if on a Q4W | Predose for Compound 9 |
| schedule of ANTIBODY Y | |
| Cycle 1 Day 22 | Anytime |
| Cycle 2 Day 1 | Preinfusion and predose of Compound 9 |
| | Immediately after ANTIBODY Y infusion |
| | (≤10 min)^{a} |
| | Immediately after retifanlimab infusion (≤10 min), if |
| | applicable |
| | 1 hour post- Compound 9 dosing (±15 min), only |
| | Compound 9 will be measured |
| | 2 hours post- Compound 9 dosing (±15 min), only |
| | Compound 9 will be measured |
| | 4 hours post- Compound 9 dosing (±30 min), only |
| | Compound 9 will be measured |
| | 6 hours post-infusion (±1 h) of ANTIBODY Y |
| Cycle 2 Day 8 | Anytime |
| Cycle 4 Day 1 | Preinfusion and predose of Compound 9 |
| | Immediately after ANTIBODY Y infusion |
| | (≤10 min)^{a} |
| | Immediately after retifanlimab infusion (≤10 min), if |
| | applicable |
| Day 1 of every other cycle | Preinfusion (only ANTIBODY Y and |
| starting at Cycle 6 Day 1 | retifanlimab PK samples will be collected at these |
| (i.e., C6D1, C8D1, C10D1, | timepoints) |
| etc) | |
| 30-day safety follow-up visit | Anytime |

^{a}Sample is to be collected before starting retifanlimab infusion if applicable.

### Antidrug Antibodies

Blood will be collected for the detection of serum anti-drug antibodies (ADAs) to 5 ANTIBODY Y or retifanlimab (if applicable) at timepoints outlined in Table F. Blood samples will be collected from the arm contralateral to the site of IV infusion. If an indwelling catheter is used, the fluid in the catheter will be removed and discarded before the collection of blood sample for ADA assessment. ADAs will be detected using a validated assay. Serum samples will be screened for antibodies binding to ANTIBODY Y 10 or retifanlimab (if applicable), and the titer of confirmed positive samples will be reported. Other analyses may be performed to verify the stability of antibodies and/or further characterize the immunogenicity.

TABLE F

| Study Visit | Timing of Sample |
|---|---|
| Cycle 1 Day 1 | Preinfusion of ANTIBODY Y and |
| | retifanlimab (if applicable) |
| Cycle 1 Day 8 | Anytime |
| Cycle 1 Day 15 if on a Q2W | Preinfusion of ANTIBODY Y |
| schedule of ANTIBODY Y | |
| Cycle 1 Day 15 if on a Q4W | Anytime |
| schedule of ANTIBODY Y | |
| Day 1 of every other cycle | Preinfusion of ANTIBODY Y and |
| starting on Cycle 2 Day 1 (ie, | retifanlimab (if applicable) |
| C2D1, C4D1, C6D1, etc) | |
| 30-day safety follow-up visit | Anytime |

### VIII. Inclusion Criteria

Participants are eligible to be included in the study only if all of the following criteria apply:
1. Ability to comprehend and willingness to sign a written ICF for the study.
2. Male or female participant aged 18 years or older inclusive at the time of signing the ICF.
3. Must be willing and able to conform to and comply with all Protocol requirements, including all scheduled visits and Protocol procedures.
4. Willingness to undergo pre- and on-treatment tumor biopsy (core or excisional).
5. For participants in TGA of Phase 1a and Phase 1b: Fresh pretreatment biopsy is required. Archival formalin-fixed paraffin embedded (FFPE) tissue (preferably at least 1 tissue block or a minimum of 6 slides) is acceptable for use in prescreening to determine CD8+ T-lymphocyte status as long as the sample is ≤12 months old. Fine-needle aspirates are not acceptable. If the participant qualifies on the basis of CD8+ T-lymphocytes in an archival biopsy tissue sample, they will still be required to undergo fresh biopsy during the screening process.
6. For participants in TGB1, TGB2 and TGC of Phase 1a and Phase 1b: Fresh pretreatment biopsy is preferred. However, archival FFPE tissue (preferably at least 1 tissue block or 20 slides, or a minimum of 15 slides) is acceptable as long as the sample is ≤12 months old. Fine needle aspirates are not acceptable.
7. Pretreatment tumor biopsy will be collected as part of prescreening.
8. Biopsies will not be required for participants in early dose escalation cohorts in each of the treatment groups in the dose-escalation Phase 1a.
9. Have CD8 T-cell-positive tumors based on evaluation of CD8+ T-lymphocyte presence by immunohistochemistry (IHC) performed on pretreatment tumor biopsy tissue. Preselection for CD8 T-cell-positive tumors, as defined by the sponsor, will be performed as part of prescreening. CD8 T-cell-positive tumors are not required for participants in early dose escalation cohorts in each of the treatment groups in the dose escalation Phase 1a.
10. Eastern Cooperative Oncology Group (ECOG) performance status 0 or 1.
11. Measurable disease according to RECIST v1.1. Tumor lesions that are located in a previously irradiated area or in an area subjected to other locoregional therapy should only be selected as target lesions if progression has been demonstrated in such lesions. It is recommended that tumor lesions selected for biopsy not be selected as target lesions.
12. Phase 1a early dose level cohorts in each of the treatment groups only: Participants with advanced or metastatic solid tumors experiencing disease progression after treatment with available therapies, including anti-PD-(L)1 therapy (if applicable), that are known to confer clinical benefit, or who are intolerant to, or ineligible for standard treatment. Prior anti-PD-(L)1 therapy should not have been discontinued because of intolerance. Locally advanced disease must not be amenable to resection with curative intent or other curative treatments or procedures.
13. Participants with SCCHN:
   a. Participants with histologically or cytologically confirmed squamous cell carcinoma of the oral cavity, oropharynx, hypopharynx, or larynx not amenable to local therapy with curative intent (surgery or radiation with or without chemotherapy).
      Note: Carcinoma of the nasopharynx, salivary gland, or nonsquamous histologies are excluded.
   b. Participants should have disease progression after treatment with available therapies, including anti-PD-(L)1 therapy (alone or as part of a combination), that are known to confer clinical benefit or who are intolerant to or ineligible for standard treatment. Prior anti-PD-(L)1 therapy should not have been discontinued because of intolerance.
14. Participants with specified GI malignancies: Histologically or cytologically confirmed advanced or metastatic CRC, GEJ cancer, HCC, PDAC, or SCAC.
   a. Participants should have disease progression after treatment with available therapies, including anti-PD-(L)1 therapy (if applicable), that are known to confer clinical benefit or who are intolerant to or ineligible for standard treatment. Prior anti-PD-(L)1 therapy should not have been discontinued because of intolerance.
15. For participants to be enrolled in cohorts including Compound 9: The ability to swallow oral medication.
16. Willingness to avoid pregnancy or fathering children based on the criteria below:
   a. Male participants with reproductive potential must agree to take appropriate precautions to avoid fathering children (with at least 99% certainty) and refrain from donating sperm from screening through 190 days after the last dose of study treatment. Permitted methods that are at least 99% effective in preventing pregnancy should be communicated to the participants and their understanding confirmed.
   b. Female participants who are woman of childbearing potential (WOCBP) must have a negative pregnancy test at screening (serum test) and before the first dose of Day 1 (urine test) and must agree to take appropriate precautions to avoid pregnancy (with at least 99% certainty) and refrain from donating oocytes from screening through 190 days after the last dose of study treatment. Permitted methods that are at least 99% effective in preventing pregnancy should be communicated to the participants and their understanding confirmed.
   c. Female participants not considered to be of childbearing potential are eligible.

### IX. Exclusion Criteria

Participants are excluded from the study if any of the following criteria apply:
1. Clinically significant cardiac disease, unstable angina, acute myocardial infarction within 6 months of Cycle 1 Day 1, and New York Heart Association Class III or IV congestive heart failure.
2. History or presence of an electrocardiogram (ECG) abnormality that, in the investigator's opinion, is clinically meaningful. For participants to be enrolled in cohorts including Compound 9, screening QT interval corrected by Fridericia (QTcF) interval >450 milliseconds (ms) is excluded; in the event that a single QT interval corrected (QTc) is >450 ms, the participant may enroll if the average QTc for the 3 ECGs is <450 ms.
3. Known active central nervous system (CNS) metastases and/or carcinomatous meningitis. Participants who have previously treated and clinically stable brain or CNS metastases (without evidence of progression by imaging for at least 4 weeks before the first dose of study treatment and any neurologic symptoms have returned to baseline), have no evidence of new or enlarging brain metastasis or CNS edema, and have not required steroids for at least 7 days before study treatment are eligible.
4. Participants who have active or inactive autoimmune disease or syndrome (e.g., rheumatoid arthritis, moderate or severe psoriasis, multiple sclerosis, inflammatory bowel disease) that has required systemic treatment in the past 2 years or who are receiving systemic therapy for an autoimmune or inflammatory disease (i.e., with use of disease modifying agents, corticosteroids, or immunosuppressive drugs). Participants with vitiligo or resolved childhood asthma/atopy, hypothyroidism stable on hormone replacement, controlled asthma, Type I diabetes, Graves' disease, or Hashimoto's disease, or with medical monitor approval, will be eligible if they meet all other eligibility criteria. Replacement therapy (e.g., thyroxine, insulin, physiologic corticosteroid replacement therapy for adrenal or pituitary insufficiency) is not considered a form of systemic treatment and is allowed.
5. Diagnosis of immunodeficiency or is receiving chronic systemic steroid therapy (doses >10 mg daily of prednisone or equivalent) or any other form of immunosuppressive therapy within 7 days before the first dose of study treatment. Use of short courses of steroids for procedure prophylaxis, inhaled or topical steroids, or systemic corticosteroids ≤10 mg/day is permitted.
6. Known additional malignancy that is progressing or requires active treatment, or history of other malignancy within 2 years of the first dose of study treatment with the exception of cured basal cell or squamous cell carcinoma of the skin, superficial bladder cancer, prostate intraepithelial neoplasm, carcinoma in situ of the cervix, or other noninvasive or indolent malignancy, or cancers from which the participant has been disease free >1 year after treatment with curative intent.
7. Participants with laboratory values at screening defined in Table G.

TABLE G

| | |
|---|---|
| Laboratory | |
| Parameter | Exclusion Criterion |

Hematology

| | |
|---|---|
| Platelets | <100 × 10⁹/L |
| Hemoglobin | <9 g/L or <5.6 mmol/L |
| | Note: Transfusion is acceptable to meet |
| | this criteria. |
| Absolute | <1 × 10⁹/L |
| neutrophil | |
| count (ANC) | |

Hepatic

| | |
|---|---|
| Alanine | ≥2.5 × upper limit of normal (ULN) |
| aminotransferase | or ≥5 × ULN for participants with liver |
| (ALT) | metastases or HCC |
| Aspartate | ≥2.5 × ULN or ≥5 × ULN for participants |
| aminotransferase | with liver metastases or HCC |
| (AST) | |
| Total bilirubin | ≥1.5 × ULN, unless conjugated bilirubin |
| | is ≤ULN (conjugated bilirubin only needs |
| | to be tested if total bilirubin exceeds the |
| | ULN). If there is no institutional ULN, |
| | then direct bilirubin must be <40% of |
| | total bilirubin. |
| Alkaline | ≥2.5 × ULN |
| phosphatase | Participants with 1) bone metastases and |
| | 2) no hepatic parenchymal metastases on |
| | screening radiographic examinations may |
| | enroll if the alkaline phosphatase |
| | is <5 × ULN. |
| | Participants with 1) bone metastases and |
| | 2) hepatic parenchymal metastases on |
| | screening radiographic examinations may |
| | enroll if the alkaline phosphatase |
| | is <5 × ULN only with medical monitor |
| | approval. |
| Albumin | <3 g/dL |

Renal

| | |
|---|---|
| Serum creatinine | <60 mL/minute based on Cockcroft- |
| clearance | Gault formula. |
| Coagulation | |
| International | >1.5 × ULN, unless on therapeutic |
| normalized | anticoagulants and stable. |
| ratio (INR) or | |
| prothrombin | |
| time (PT) | |
| Activated partial | >1.5 × ULN |
| thromboplastin | |
| time (aPTT) | |

8. Has not recovered to ≤Grade 1 from toxic effects of prior therapy (including prior immunotherapy) and/or complications from prior surgical intervention before starting study treatment. Participants with stable chronic conditions (≤Grade 2) not expected to resolve (such as neuropathy and alopecia) are exceptions and may enroll.
9. Evidence of interstitial lung disease, history of interstitial lung disease, or active noninfectious pneumonitis.
10. Immune-related toxicity during prior immune therapy for which permanent discontinuation of therapy is recommended (per product label or consensus guidelines), or any immune-related toxicity requiring intensive or prolonged immunosuppression to manage with the exception of endocrinopathy that is well-controlled on replacement hormones.
11. Prior treatment with any adenosine pathway targeting drugs (e.g., A2A receptor and/or A2B receptor antagonists, anti-CD38, anti-CD39, anti-CD-73/CD73 antagonists). Participants to be enrolled into the dose escalation (Phase 1a) who were treated with Compound 9 monotherapy may be allowed to enroll following medical monitor approval.
12. Any prior chemotherapy, biological therapy, or targeted therapy to treat the participant's disease within 5 half-lives or 28 days (whichever is shorter) before the first dose of study treatment. Participants receiving denosumab are eligible for enrollment. No wash-out period following anti-PD-(L)1 therapy is required (however, at least 1 dosing cycle of the last prior anti-PD-(L)1 therapy must be completed before the first dose of study treatment). Any prior radiation therapy within 28 days before the first dose of study treatment. Participants who have received radiation therapy must have recovered from all radiation-related toxicities, not require corticosteroids for this purpose, and not have had radiation pneumonitis as a result of treatment.
13. Undergoing treatment with another investigational medication or having been treated with an investigational medication within 5 half-lives or 28 days (whichever is shorter) before the first dose of study treatment. The medical monitor should be contacted in the event a participant received any treatment for signs or symptoms of coronavirus disease 2019 (COVID-19).
14. For participants to be enrolled in cohorts including Compound 9: Concomitant treatment with strong Cytochrome P450 3A4 (CYP3A4) inhibitors or inducers. A washout period of ≥14 days before the first dose of Compound 9 is required for enrollment into the study for prior treatment with strong CYP3A4 inducers. A washout period of ≥5 half-lives before the first dose of Compound 9 is required for enrollment into the study for prior treatment with strong CYP3A4 inhibitors.
15. Receipt of a live virus vaccine within 30 days of the first dose of study treatment. Examples of live vaccines include, but are not limited to, the following: measles, mumps, rubella, chicken pox/zoster, yellow fever, rabies, Bacillus Calmette-Guerin, and typhoid vaccine. Seasonal influenza vaccines for injection are generally killed virus vaccines and are allowed; however, intranasal influenza vaccines are live-attenuated vaccines and are not allowed.
16. Infection requiring parenteral antibiotics, antivirals, or antifungals within 1 week of the first dose of study treatment.
17. Known or suspected severe acute respiratory syndrome-coronavirus-2 (SARS-CoV-2) infection at the time of enrollment. Participants who failed screening due to SARS-CoV-2 infection may be reassessed for eligibility in the study (ie, repeat the screening process) following a negative SARS-CoV-2 test and clinical recovery as per investigator's evaluation.
18. Active hepatitis B virus (HBV) or hepatitis C virus (HCV) infection that requires treatment. HBV-DNA and HCV-RNA must be undetectable. Participants who have cleared a prior HBV infection (defined as hepatitis B surface antigen (HBsAg) negative), HBsAg antibody positive, and anti-hepatitis B core (anti-HBc antibody positive) are eligible for the study. For participants with a cleared prior HBV infection, HBV prophylaxis should be considered per the investigator's discretion. Monitor for HBV reactivation every 3 cycles by performing HBV viral load and HBsAg serology testing. Additional viral serologic testing may be performed at the investigator's discretion. Participants with no prior history of HBV infection who have been vaccinated against HBV and who have a positive antibody against HBsAg as the only evidence of prior exposure may participate in the study. HCV antibody-positive participants who received and completed treatment for hepatitis C that was intended to eradicate the virus may participate if HCV-RNA levels are undetectable. HCV antibody testing is allowed for screening purposes in countries where HCV-RNA is not part of standard of care (SoC). In these cases, HCV antibody-positive participants will be excluded.
19. Known history of HIV (HIV 1/2 antibodies).
20. History of organ transplant, including allogeneic stem-cell transplantation or CAR-T cell therapy.
21. Known hypersensitivity or severe reaction to any component of study drug(s) or formulation components.
22. For participants to be enrolled in cohorts including Compound 9: Inability to swallow food or any concomitant condition of the upper GI tract that precludes administration of oral medications.
23. Is pregnant or breastfeeding.
24. Any condition that would, in the investigator's judgment, interfere with full participation in the study, including administration of study treatment and attending required study visits; pose a significant risk to the participant; or interfere with interpretation of study data.
25. For studies conducted in France: The following participants are excluded in France: vulnerable populations according to article L.1121-6 of the French Public Health Code and adults under legal protection or who are unable to express their consent per article L.1121-8 of the French Public Health Code.

### X. Objectives and Endpoints

TABLE H

| | |
|---|---|
| Objectives | Endpoints |

Primary

| | |
|---|---|
| To evaluate the safety, tolerability, and | DLT |
| DLTs and determine the RDE of | AEs, assessed by physical examinations, |
| ANTIBODY Y as monotherapy and of | evaluating changes in vital signs and |
| combination treatments of ANTIBODY Y | ECGs, and through clinical laboratory |
| with retifanlimab and/or Compound 9 in | blood sample evaluations. |
| participants with advanced solid tumors. | Impact on study treatment, assessed by |
| | treatment interruptions, dose reductions, |
| | and withdrawal of study treatment due |
| | to AEs. |

Secondary

| | |
|---|---|
| To evaluate the pharmacokinetic of | PK parameters for ANTIBODY Y |
| ANTIBODY Y as monotherapy or in | including maximum observed plasma |
| combination with retifanlimab and/or | concentration (Cₘₐₓ), tₘₐₓ, concentration at |
| Compound 9 in participants with | the end of the dosing interval (Cₜₐᵤ), area |
| advanced solid tumors. | under the plasma concentration-time |
| | curve (AUC) total clearance (CL), volume |
| | of distribution (V_{z}), and half-life (t_{1/2}) as |
| | deemed appropriate. |
| To evaluate the intratumoral | Blockade of CD73 enzymatic activity. |
| pharmacodynamic activity of | |
| ANTIBODY Y as monotherapy in | |
| participants with advanced solid tumors. | |
| To determine the preliminary efficacy of | Objective response: CR or PR, as |
| ANTIBODY Y as monotherapy or in | determined by investigator by |
| combination with retifanlimab and/or | radiographic disease assessment |
| Compound 9 in terms of objective | according to RECIST v1.1. |
| response rate (ORR), disease control rate | Disease control: CR or PR, or stable |
| (DCR), and duration of response (DOR) | disease (SD) as determined by |
| in participants with selected advanced | investigator by radiographic disease |
| solid tumors. | assessment according to RECIST v1.1. |
| | Duration of response: time from earliest |
| | date of disease response (CR or PR) |
| | until earliest date of disease progression |
| | as determined by investigator by |
| | radiographic disease assessment |
| | according to RECIST v1.1, or death due |
| | to any cause, if occurring sooner than |
| | progression. |

Exploratory

| | |
|---|---|
| To evaluate the PK of retifanlimab in | PK parameters for retifanlimab including |
| combination with ANTIBODY Y (double | Cₘₐₓ, tₘₐₓ, Cₜₐᵤ, AUC, V_{z}, and t_{1/2} as |
| combination) or ANTIBODY Y and | deemed appropriate. |
| Compound 9 (triple combination) in | |
| participants with advanced solid tumors. | |
| To evaluate the PK of Compound 9 in | PK parameters for Compound 9 including |
| combination with ANTIBODY Y | Cₘₐₓ, tₘₐₓ, Cₜₐᵤ, AUC, apparent total |
| (doublet) or ANTIBODY Y and | clearance (CL/F), apparent volume of |
| retifanlimab (triplet) in participants with | distribution (V_{z}/F), and t_{1/2} as deemed |
| advanced solid tumors. | appropriate. |
| To explore biomarkers that predict | Blood and/or tumor analytes and other |
| pharmacologic activity and/or correlate | relevant biomarkers with respect to safety |
| with clinical safety or efficacy. | and efficacy outcome measures. |
| To assess the immunogenicity of | Immunogenicity, defined as the |
| ANTIBODY Y and retifanlimab in | occurrence of specific ADAs to |
| participants with advanced solid tumors. | ANTIBODY Y or retifanlimab. |

### Example A: Activity of A2A/A2B Inhibitors

### I. A2A Tag-lite® HTRF Assay

Assays were conducted in black low volume 384-well polystyrene plates (Greiner 784076-25) in a final volume of 10 μL. Test compounds were first serially diluted in DMSO and 100 nL added to the plate wells before the addition of other reaction components. The final concentration of DMSO was 1%. Tag-lite® Adenosine A2A labeled cells (CisBio C1TT1A2A) were diluted 1:5 into Tag-lite buffer (CisBio LABMED) and spun 1200 g for 5 mins. The pellet was resuspended at a volume 10.4× the initial cell suspension volume in Tag-lite buffer, and Adenosine A2A Receptor Red antagonist fluorescent ligand (CisBio L0058 RED) added at 12.5 nM final concentration. 10 μL of the cell and ligand mix was added to the assay wells and incubated at room temperature for 45 minutes before reading on a PHERAstar FS plate reader (BMG Labtech) with HTRF 337/620/665 optical module. Percent binding of the fluorescent ligand was calculated; where 100 nM of A2A antagonist control ZM 241385 (Tocris 1036) displaces the ligand 100% and 1% DMSO has 0% displacement. The % binding data versus the log of the inhibitor concentration was fitted to a one-site competitive binding model (GraphPad Prism version 7.02) where the ligand constant=12.5 nM and the ligand Kd=1.85 nM. The Ki data obtained via this method are shown in Table 6.

II Adenosine A2B Receptor cyclic AMP GS Assay

Stably transfected HEK-293 cells expressing the human adenosine A2B receptor (Perkin Elmer) were maintained in MEM culture medium with 10% FBS and 100 μg/mL Geneticin (Life Technologies). 18 to 24 hours prior to assay, geneticin was removed from culture. The cisbio cAMP-GS Dynamic kit utilizing the FRET (Fluorescence Resonance Energy Transfer) technology was used to measure cAMP accumulation in the cells. Compounds of the present disclosure at an appropriate concentration were mixed with 10000 cells/well in white 96 well half area plates (Perkin Elmer) for 30 min at RT gently shaking. Agonist, NECA (R&D Technologies) at 12 nM was added to each well for 60 min at RT gently shaking. Detection reagents, d2-labeled cAMP (acceptor) and anti-cAMP cryptate (donor) were added to each well for 60 min at RT gently shaking. Plates were read on Pherastar (BMG Labtech), fluorescence ratio 665/620 was calculated and EC₅₀ determination was performed by fitting the curve of percent of control versus the log of the compound concentration using GraphPad Prism. The EC₅₀ data obtained via this method are shown in Table 6.

TABLE 6
The A_{2A—}Ki data (Example A(I)) and A_{2B—}cAMP_EC₅₀
data (Example A(II)) are provided below.

| Comp. | A_{2A—}Ki | A_{2B—}cAMP_EC₅₀ |
|---|---|---|
| No. | (nM) | (nM) |
| 1 | † | † |
| 2 | † | † |
| 3 | † | † |
| 4 | † | † |
| 5 | † | † |
| 6 | † | † |
| 7 | † | † |
| 8 | † | †† |
| 9 | † | † |
| 10 | † | † |
| 11 | † | † |
| 12 | † | †† |
| 13 | † | † |
| 14 | † | † |
| 15 | † | † |
| 16 | † | † |
| 17 | † | †† |
| 18 | † | † |
| 19 | † | † |
| 20 | † | † |
| 21 | † | † |

† indicates A_{2A—}Kᵢ or A_{2B—}cAMP_EC₅₀ ≤ 10 nM,
†† indicates A_{2A—}Kᵢ or A_{2B—}cAMP_EC₅₀ > 10 nM but ≤ 100 nM,
††† indicates A_{2A—}Kᵢ or A_{2B—}cAMP_EC₅₀ > 100 nM but ≤ 1 μM,
†††† indicates A_{2A—}Kᵢ or A_{2B—}cAMP_EC₅₀ is greater than 1 μM.

### Example A1: Synthesis of 3-(5-Amino-2-(pyridin-2-ylmethyl)-8-(pyrimidin-4-yl)-[1,2,4]triazolo[1,5-c]pyrimidin-7-yl)benzonitrile

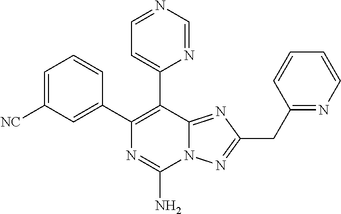 [SMILES: N#Cc1cccc(-c2nc(N)n3nc(Cc4ccccn4)nc3c2-c2ccncn2)c1]

### Step 1: 3-(2-Amino-6-chloropyrimidin-4-yl)benzonitrile

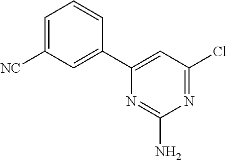 [SMILES: N#Cc1cccc(-c2cc(Cl)nc(N)n2)c1]

A mixture of 4,6-dichloropyrimidin-2-amine (2.5 g, 15.2 mmol), (3-cyanophenyl)boronic acid (2.02 g, 13.7 mmol), tetrakis(triphenylphosphine)palladium(0) (1.06 g, 0.92 mmol) and sodium carbonate (3.23 g, 30.5 mmol) in 1,4-dioxane (60 mL), and water (5 mL) was degassed with nitrogen, then the resulting mixture was heated and stirred at 60° C. for two days. After cooled to room temperature (r.t.), the mixture was concentrated, diluted with water, and extracted with DCM (30 mL×3). The combined organic layers were dried over MgSO₄, filtered, and concentrated. The resulting residue was purified by flash chromatography on a silica gel column eluting with 8% EtOAc in dichloromethane to afford the desired product. LCMS calculated for C₁₁H₈ClN₄ (M+H)⁺: 231.0. Found: 231.0.

### Step 2: 2-(Pyridin-2-yl)acetohydrazide

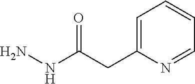 [SMILES: NNC(=O)Cc1ccccn1]

Hydrazine (4.15 mL, 132 mmol) was added to a ethanol (66 mL) solution of methyl 2-(pyridin-2-yl)acetate (10 g, 66.2 mmol) at r.t. The mixture was heated and stirred at 85° C. for 4 h, and then cooled to r.t. White solid was formed upon standing, which was collected via filtration and used in next step without further purification. LCMS calculated for C₇H₁₀N₃O (M+H)⁺: 152.1. Found: 152.0.

### Step 3: 3-(5-Amino-2-(pyridin-2-ylmethyl)-[1,2,4]triazolo[1,5-c]pyrimidin-7-yl)benzonitrile

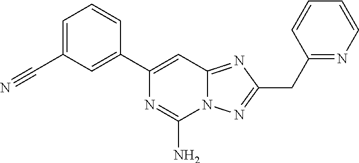 [SMILES: N#Cc1cccc(-c2cc3nc(Cc4ccccn4)nn3c(N)n2)c1]

2-(pyridin-2-yl)acetohydrazide (2.62 g, 17.34 mmol) was added to a ethanol (35 mL) solution of 3-(2-amino-6-chloropyrimidin-4-yl)benzonitrile (4.00 g, 17.34 mmol) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO₃ solution. The resulting precipitate was collected by filtration, and dried to obtain the desired product as a brown solid. LCMS calculated for C₁₈H₁₄N₇ (M+H)⁺: 328.1; found 328.1.

### Step 4: 3-(5-Amino-8-bromo-2-(pyridin-2-ylmethyl)-[1,2,4]triazolo[1,5-c]pyrimidin-7-yl)benzonitrile

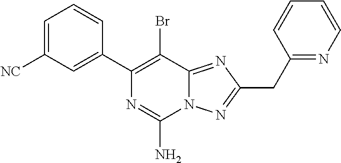 [SMILES: N#Cc1cccc(-c2nc(N)n3nc(Cc4ccccn4)nc3c2Br)c1]

To a mixture of 3-(5-amino-2-(pyridin-2-ylmethyl)-[1,2,4]triazolo[1,5-c]pyrimidin-7-yl)benzonitrile (2 g, 6.11 mmol) in DMF (12 mL) at −30° C. was added NBS (1.09 g, 6.11 mmol) portion-wise. The reaction mixture was allowed to slowly warm to 0° C., resulting a homogenous solution. After stirring at 0° C. for 1 h, the reaction mixture was diluted with saturated NaHCO₃ solution and the resulting solid was collected by filtration. The solid was then purified by flash chromatography on a silica gel column eluting with 0 to 10% MeOH in DCM to afford the desired product. LCMS calculated for C₁₈H₁₃BrN₇ (M+H)⁺: 406.0; found 406.0.

### Step 5: 3-(5-Amino-2-(pyridin-2-ylmethyl)-8-(pyrimidin-4-yl)-[1,2,4]triazolo[1,5-c]pyrimidin-7-yl)benzonitrile

Pd(Ph₃P)₄ (284 mg, 0.246 mmol) was added to a mixture of 4-(tributylstannyl)pyrimidine (1090 mg, 2.95 mmol), 3-(5-amino-8-bromo-2-(pyridin-2-ylmethyl)-[1,2,4]triazolo[1,5-c]pyrimidin-7-yl)benzonitrile (1000 mg, 2.46 mmol), and copper(I) chloride (244 mg, 2.46 mmol) in 1,4-dioxane (12 mL). The reaction mixture was purged with N2 and stirred at 80° C. for 7 h. The resulting mixture was cooled to r.t., concentrated, diluted with DCM (50 mL) and washed with saturated NH₄₀H solution. The organic layer was dried over Na₂SO₄, concentrated, and purified by preparative LCMS (pH 2, acetonitrile/water with TFA) to afford the product as a TFA salt. LCMS calculated for C₂₂H₁₆N₉ (M+H)⁺: 406.2; found 406.2. ¹H NMR (500 MHz, DMSO) δ 8.95 (s, 1H), 8.83 (d, J=5.3 Hz, 1H), 8.59 (d, J=5.1 Hz, 1H), 7.96 (m, 1H), 7.88 (d, J=5.1 Hz, 1H), 7.82 (d, J=7.6 Hz, 1H), 7.76 (s, 1H), 7.60-7.53 (m, 2H), 7.53-7.48 (m, 1H), 7.48-7.42 (m, 1H), 4.49 (s, 2H).

### Example A2: Synthesis of 3-(5-Amino-2-((2,6-difluorophenyl)(hydroxy)methyl)-8-(pyrimidin-4-yl)-[1,2,4]triazolo[1,5-c]pyrimidin-7-yl)benzonitrile

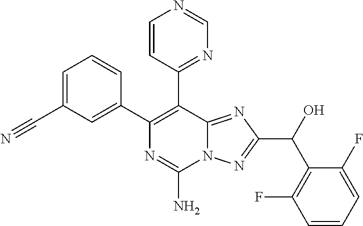 [SMILES: N#Cc1cccc(-c2nc(N)n3nc(C(O)c4c(F)cccc4F)nc3c2-c2ccncn2)c1]

### Step 1: Methyl 2-(2, 6-difluorophenyl)-2-hydroxyacetate

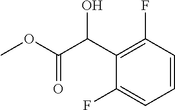

Concentrated sulfuric acid (1.42 mL, 27 mmol) was added to a methanol (45 mL) solution of 2,6-difluoromandelic acid (5 g, 27 mmol) at 0° C. The mixture was stirred at r.t. for 4 h before being concentrated. To the resulting slurry was added saturated NaHCO₃ solution (30 mL). The resulting mixture was extracted with DCM (3×20 mL). The combined organic layers were washed with water, dried over Mg₂SO₄, filtered, and concentrated to afford the crude product, which was used in the next step without further purification. LC-MS calculated for C₁₁H₁₂F₂NO₃ (M+H+MeCN)⁺: m/z=244.1; found 244.2.

### Step 2: 3-(5-Amino-2-((2,6-difluorophenyl)(hydroxy)methyl)-8-(pyrimidin-4-yl)-[1,2,4]triazolo[1,5-c]pyrimidin-7-yl)benzonitrile

This compound was prepared using similar procedures as described for Example A1, with methyl 2-(2,6-difluorophenyl)-2-hydroxyacetate replacing methyl 2-(pyridin-2-yl)acetate in Step 2. The two enantiomers were separated by chiral SFC using a Phenomenex Lux Cellulose-1 column (21.2×250 mm, 5 m particle size) eluting with an isocratic mobile phase 25% MeOH in CO₂ with a flow rate of 80 mL/minute. Peak 1 was isolated, and further purified by prep-LCMS (pH=2, MeCN/water with TFA) to give the desired product as a TFA salt. LC-MS calculated for C₂₃H₁₅F₂N₈O (M+H)⁺: m/z=457.1; found 457.1. ¹H NMR (500 MHz, DMSO) δ 8.94 (d, J=1.3 Hz, 1H), 8.81 (d, J=5.2 Hz, 1H), 7.85 (dd, J=5.3, 1.4 Hz, 1H), 7.81 (dt, J=7.4, 1.5 Hz, 1H), 7.76 (t, J=1.7 Hz, 1H), 7.55 (dt, J=7.8, 1.5 Hz, 1H), 7.49 (t, J=7.8 Hz, 1H), 7.44 (tt, J=8.4, 6.4 Hz, 1H), 7.09 (t, J=8.3 Hz, 2H), 6.27 (s, 1H).

### Example A3: Synthesis of 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile (Compound 3A) and 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile (Compound 3B)

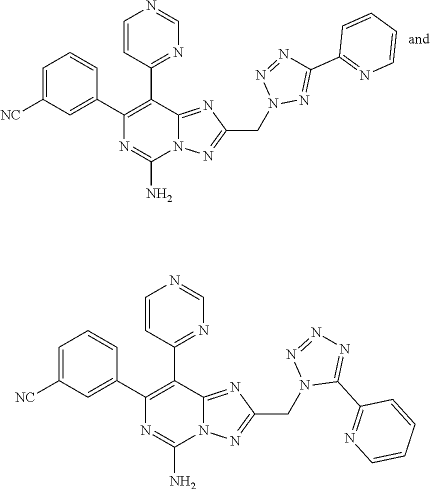 [SMILES: N#Cc1cccc(-c2nc(N)n3nc(Cn4nnc(-c5ccccn5)n4)nc3c2-c2ccncn2)c1 ; N#Cc1cccc(-c2nc(N)n3nc(Cn4nnnc4-c4ccccn4)nc3c2-c2ccncn2)c1]

### Step 1: 3-(5-Amino-2-(hydroxymethyl)-[1,2,4]triazolo[1,5-c]pyrimidin-7-yl)benzonitrile

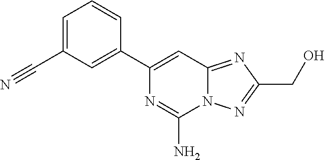 [SMILES: N#Cc1cccc(-c2cc3nc(CO)nn3c(N)n2)c1]

2-Hydroxyacetohydrazide (2.34 g, 26.01 mmol) was added to a ethanol (35 mL) solution of 3-(2-amino-6-chloropyrimidin-4-yl)benzonitrile (4.00 g, 17.34 mmol) (Example A1, Step 1) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO₃ solution. The resulting precipitate was collected by filtration, and dried to obtain the desired product as a brown solid. LCMS calculated for C₁₃H₁₁N₆O (M+H)+: 267.1; found 267.1.

### Step 2: 3-(5-Amino-8-bromo-2-(hydroxymethyl)-[1,2,4]triazolo[1,5-c]pyrimidin-7-yl)benzonitrile

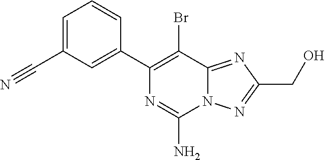 [SMILES: N#Cc1cccc(-c2nc(N)n3nc(CO)nc3c2Br)c1]

To a mixture of 3-(5-amino-2-(hydroxymethyl)-[1,2,4]triazolo[1,5-c]pyrimidin-7-yl)benzonitrile (1.0 g, 3.76 mmol) in DMF (12 mL) at −30° C. was added NBS (0.67 g, 3.76 mmol) portion-wise. The reaction mixture was allowed to slowly warm to 0° C., resulting a homogenous solution. After stirring at 0° C. for 1 h, the reaction mixture was diluted with saturated NaHCO₃ solution and the desired product was collected by filtration and dried. LCMS calculated for C₁₃H₁₀BrN₆O (M+H)+: 345.0; found 345.0.

### Step 3: 3-(5-Amino-2-(hydroxymethyl)-8-(pyrimidin-4-yl)-[1,2,4]triazolo[1,5-C]pyrimidin-7-yl)benzonitrile

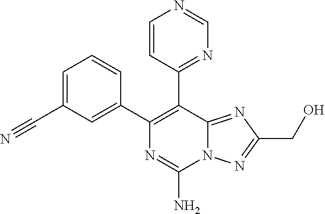 [SMILES: N#Cc1cccc(-c2nc(N)n3nc(CO)nc3c2-c2ccncn2)c1]

Tetrakis(triphenylphosphine)palladium(0) (0.067 g, 0.058 mmol) was added to a mixture of 4-(tributylstannyl)pyrimidine (0.321 g, 0.869 mmol), 3-(5-amino-8-bromo-2-(hydroxymethyl)-[1,2,4]triazolo[1,5-c]pyrimidin-7-yl)benzonitrile (0.20 g, 0.579 mmol), CsF (0.176 g, 1.159 mmol), and copper(I)iodide (0.022 g, 0.116 mmol) in 1,4-dioxane (5.0 mL). The reaction mixture was purged with N₂ and stirred at 80° C. for 7 h. The resulting mixture was cooled to r.t., concentrated and purified by flash column chromatopraphy eluting with 0% to 10% methanol in DCM to afford the product. LC-MS calculated for C₁₇H₁₃N₈O (M+H)+: 345.1; found 345.1.

### Step 4: 3-(5-Amino-2-(chloromethyl)-8-(pyrimidin-4-yl)-[1,2,4]triazolo[1,5-c]pyrimidin-7-yl)benzonitrile

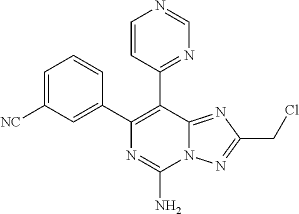 [SMILES: N#Cc1cccc(-c2nc(N)n3nc(CCl)nc3c2-c2ccncn2)c1]

To a mixture of 3-(5-amino-2-(hydroxymethyl)-8-(pyrimidin-4-yl)-[1,2,4]triazolo[1,5-c]pyrimidin-7-yl)benzonitrile (0.1 g, 0.290 mmol) in acetonitrile (10 ml) was added thionyl chloride (0.212 ml, 2.90 mmol) at r.t. The reaction mixture was stirred at r.t. for 5 h, concentrated, and purified by flash chromatography eluting with 0% to 5% methanol in DCM to afford the product. LC-MS calculated for C₁₇H₁₂ClN₈ (M+H)⁺: 363.1; found 363.1.

### Step 5: Mixture of 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile (Compound 3A) and 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile (Compound 3B)

A mixture of 3-(5-amino-2-(chloromethyl)-8-(pyrimidin-4-yl)-[1,2,4]triazolo[1,5-c]pyrimidin-7-yl)benzonitrile (10 mg, 0.028 mmol), 2-(1H-tetrazol-5-yl)pyridine (8.1 mg, 0.055 mmol) and Cs₂CO₃ (20.7 mg, 0.064 mmol) in DMF (1 mL) was stirred at 100° C. for 10 min. The reaction mixture was then cooled to r.t., diluted with methanol (4 mL), and purified by preparative LC-MS (pH 2, acetonitrile/water with TFA) to afford the product as a TFA salt. LCMS calculated for C₂₃H₁₆N₁₃ (M+H)⁺: 474.2; found 474.2.

Compound 3A: ¹H NMR (500 MHz, DMSO) δ 8.99 (d, J=1.4 Hz, 1H), 8.85 (d, J=5.3 Hz, 1H), 8.80-8.71 (m, 1H), 8.71-8.39 (b, 2H), 8.18 (d, J=7.7, 1.1 Hz, 1H), 8.04 (t, J=7.8, 1.8 Hz, 1H), 7.85 (m, 2H), 7.80-7.76 (m, 1H), 7.62-7.55 (m, 2H), 7.53 (t, J=7.8 Hz, 1H), 6.39 (s, 2H).

### Example A4: Synthesis of 3-(5-Amino-2-((3-methylpyridin-2-yl)methoxy)-8-(pyrimidin-4-yl)-[1,2,4]triazolo[1,5-c]pyrimidin-7-yl)benzonitrile

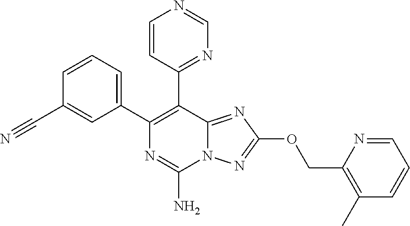 [SMILES: Cc1cccnc1COc1nc2c(-c3ccncn3)c(-c3cccc(C#N)c3)nc(N)n2n1]

### Step 1: 6-Chloro-N²,N²-bis(4-methoxybenzyl)pyrimidine-2,4-diamine

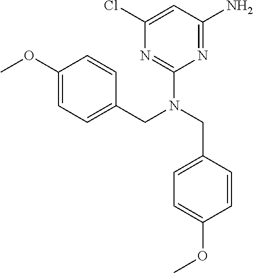 [SMILES: COc1ccc(CN(Cc2ccc(OC)cc2)c2nc(N)cc(Cl)n2)cc1]

To a solution of 2,6-dichloropyrimidin-4-amine (5.0 g, 31 mmol) in 2-propanol (31 mL) was added N,N-diisopropylethylamine (6.4 mL, 37 mmol) and bis(4-methoxybenzyl)amine (7.9 g, 31 mmol). The resulting solution was stirred at 100° C. for 16 h, cooled to r.t., diluted with water (100 mL), and extracted with EtOAc (100 mL). The organic layer was washed with water and brine, dried over anhydrous sodium sulfate, and concentrated to yield the crude product, which was used in the next step without further purification. LC-MS calculated for C₂₀H₂₂ClN₄O₂ (M+H)⁺: 385.1; found 385.1.

### Step 2: 7-Chloro-N⁵,N⁵-bis(4-methoxybenzyl)-[1,2,4]triazolo[1,5-c]pyrimidine-2,5-diamine

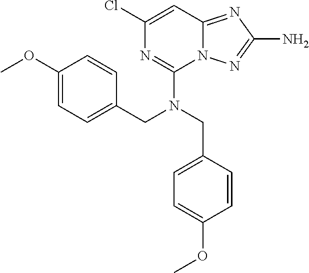 [SMILES: COc1ccc(CN(Cc2ccc(OC)cc2)c2nc(Cl)cc3nc(N)nn23)cc1]

O-ethyl carbonisothiocyanatidate (3.1 mL, 26 mmol) was added to a 1,4-dioxane (5.0 mL) solution of 6-chloro-N²,N²-bis(4-methoxybenzyl)pyrimidine-2,4-diamine (1.0 g, 2.6 mmol) at r.t. The reaction mixture was then stirred at 90° C. overnight, cooled to r.t., and concentrated. The resulting material was dissolved in methanol (12 mL) and ethanol (12 mL), and N,N-diisopropylethylamine (0.91 mL, 5.2 mmol) was added, followed by hydroxylamine hydrochoride (0.54 g, 7.8 mmol). The reaction mixture was stirred at 45° C. for 2 h, cooled to r.t., and concentrated. The resulting material was taken into EtOAc, washed with water, dried over anhydrous sodium sulfate, and concentrated. The crude material was then purified by silica gel chromatography eluting with 0% to 50% EtOAc in hexanes to afford the product. LC-MS calculated for C₂₁H₂₂CIN₆O₂ (M+H)⁺: 425.1; found 425.2.

### Step 3: 3-(2-Amino-5-(bis(4-methoxybenzyl)amino)-[1,2,4]triazolo[1,5-c]pyrimidin-7-yl)benzonitrile

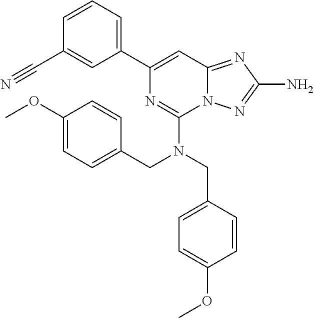 [SMILES: COc1ccc(CN(Cc2ccc(OC)cc2)c2nc(-c3cccc(C#N)c3)cc3nc(N)nn23)cc1]

Chloro(2-dicyclohexylphosphino-2′,4′,6′-tri-i-propyl-1,1′-biphenyl)(2′-amino-1,1′-biphenyl-2-yl) palladium(II) (330 mg, 0.42 mmol) was added to a mixture of (3-cyanophenyl)boronic acid (460 mg, 3.2 mmol), 7-chloro-N⁵,N⁵-bis(4-methoxybenzyl)-[1,2,4]triazolo[1,5-c]pyrimidine-2,5-diamine (890 mg, 2.1 mmol), and sodium carbonate (890 mg, 8.4 mmol) in 1,4-dioxane (8.8 mL) and water (1.8 mL). The mixture was purged with N₂ and stirred at 95° C. overnight. The reaction mixture was then cooled to r.t., concentrated, and purified by silica gel chromatography eluting with 0% to 50% EtOAc in DCM to afford the desired product. LC-MS calculated for C₂₈H₂₆N₇O₂ (M+H)⁺: 492.2; found 492.2.

### Step 4: 3-(2-Amino-5-(bis(4-methoxybenzyl)amino)-8-bromo-[1,2,4]triazolo[1,5-c]pyrimidin-7-yl)benzonitrile

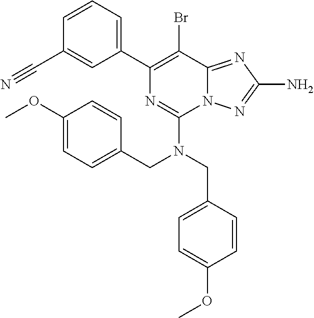 [SMILES: COc1ccc(CN(Cc2ccc(OC)cc2)c2nc(-c3cccc(C#N)c3)c(Br)c3nc(N)nn23)cc1]

To a solution of 3-(2-amino-5-(bis(4-methoxybenzyl)amino)-[1,2,4]triazolo[1,5-c]pyrimidin-7-yl)benzonitrile (330 mg, 0.66 mmol) in DMF (1.4 mL) was slowly added NBS (120 mg, 0.66 mmol) at 0° C. The reaction mixture was then stirred at r.t. for 30 min before water (10 mL) was added. The resulting solid was collected by filtration, and dried to obtain the desired product. LC-MS calculated for C₂₈H₂₅BrN₇O₂ (M+H)⁺: m/z=570.1; found 570.2.

### Step 5: 3-(2-Amino-5-(bis(4-methoxybenzyl)amino)-8-(pyrimidin-4-yl)-[1,2,4]triazolo[1,5-c]pyrimidin-7-yl)benzonitrile

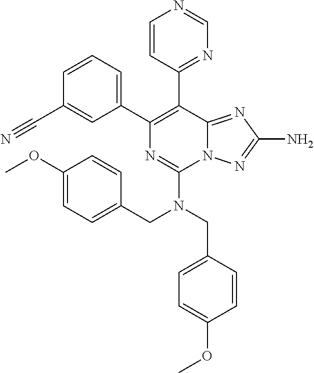 [SMILES: COc1ccc(CN(Cc2ccc(OC)cc2)c2nc(-c3cccc(C#N)c3)c(-c3ccncn3)c3nc(N)nn23)cc1]

A mixture of 3-(2-amino-5-(bis(4-methoxybenzyl)amino)-8-bromo-[1,2,4]triazolo[1,5-c]pyrimidin-7-yl)benzonitrile (350 mg, 0.61 mmol), 4-(tributylstannyl)pyrimidine (210 μL, 0.67 mmol), tetrakis(triphenylphosphine)palladium(0) (70 mg, 0.060 mmol), copper(I) iodide (23 mg, 0.12 mmol) and cesium fluoride (180 mg, 1.2 mmol) in dioxane (4.7 mL) was heated and stirred at 140° C. for 30 min in a microwave reactor. The reaction mixture was then cooled to r.t., filtered through a Celite plug (washed with DCM), and concentrated. The resulting material was purified by silica gel column chromatography eluting with 0-20% MeOH/DCM to give the desired product. LC-MS calculated for C₃₂H₂₈N₉O₂ (M+H)⁺: m/z=570.2; found 570.3.

### Step 6: 3-(5-(Bis(4-methoxybenzyl)amino)-2-bromo-8-(pyrimidin-4-yl)-[1,2,4]triazolo[1,5-c]pyrimidin-7-yl)benzonitrile

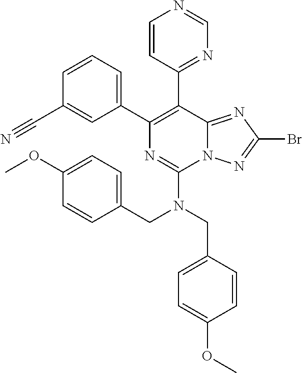 [SMILES: COc1ccc(CN(Cc2ccc(OC)cc2)c2nc(-c3cccc(C#N)c3)c(-c3ccncn3)c3nc(Br)nn23)cc1]

To a mixture of copper(II) bromide (91 mg, 0.407 mmol) and tert-butyl nitrite (0.054 ml, 0.407 mmol) in acetonitrile (3 mL) under nitrogen at 50° C. was added dropwise 3-(2-amino-5-(bis(4-methoxybenzyl)amino)-8-(pyrimidin-4-yl)-[1,2,4]triazolo[1,5-c]pyrimidin-7-yl)benzonitrile (100 mg, 0.203 mmol) in acetonitrile (3 mL). The mixture was stirred at 50° C. for 2 hours. After cooling to room temperature, 1 N aqueous NH₄₀H solution (20 mL) was added, and the mixture was extracted three times with CH₂C₁₂ (20 mL). The combined organic layers were dried over sodium sulfate, filtered and concentrated. The crude material was purified by silica gel column chromatography eluting with 50-100% ethyl acetate/hexane to give the desired product. LC-MS calculated for C₃₂H₂₆BrN₈O₂ (M+H)⁺: m/z=633.1; found 633.2.

### Step 7: 3-(5-Amino-2-((3-methylpyridin-2-yl)methoxy)-8-(pyrimidin-4-yl)-[1,2,4]triazolo[1,5-c]pyrimidin-7-yl)benzonitrile

A suspension of sodium hydride (60% in mineral oil, 3.8 mg, 0.095 mmol), 3-(5-(bis(4-methoxybenzyl)amino)-2-bromo-8-(pyrimidin-4-yl)-[1,2,4]triazolo[1,5-c]pyrimidin-7-yl)benzonitrile (20 mg, 0.032 mmol) and (3-methylpyridin-2-yl)methanol (9.1 μL, 0.095 mmol) in 1,4-dioxane (1 mL) was heated and stirred at 110° C. under nitrogen overnight. The reaction mixture was then cooled to rt, concentrated, and added TFA (1.0 mL). The resulting mixture was then stirred at 110° C. for 30 min, cooled to rt, diluted with acetonitrile, filtered and purified by preparative LC-MS (pH 2, acetonitrile/water with TFA) to give desired product as a TFA salt. LC-MS calculated for C₂₃H₁₈N₉O (M+H)⁺: m/z=436.2; found 436.2. ¹H NMR (600 MHz, DMSO) δ 8.97 (d, J=1.4 Hz, 1H), 8.88 (d, J=5.2 Hz, 1H), 8.58-8.52 (m, 1H), 7.97 (d, J=7.8 Hz, 1H), 7.88 (dd, J=5.4, 1.4 Hz, 1H), 7.85 (dt, J=7.5, 1.5 Hz, 1H), 7.78 (t, J=1.8 Hz, 1H), 7.60-7.54 (m, 2H), 7.53 (t, J=7.8 Hz, 1H), 5.69 (s, 2H), 2.48 (s, 3H).

### Example A5: Synthesis of 3-(5-Amino-2-(hydroxy(phenyl)methyl)-[1,2,4]triazolo[1,5-c]pyrimidin-7-yl)benzonitrile

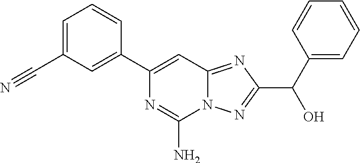 [SMILES: N#Cc1cccc(-c2cc3nc(C(O)c4ccccc4)nn3c(N)n2)c1]

### Step 1: 3-(2-Amino-6-chloropyrimidin-4-yl)benzonitrile

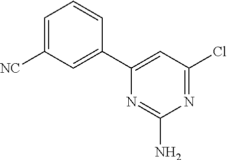 [SMILES: N#Cc1cccc(-c2cc(Cl)nc(N)n2)c1]

A mixture of 4,6-dichloropyrimidin-2-amine (2.5 g, 15.24 mmol), (3-cyanophenyl)boronic acid (2.016 g, 13.72 mmol), tetrakis(triphenylphosphine)palladium(0) (1.057 g, 0.915 mmol) and sodium carbonate (3.23 g, 30.5 mmol) in 1,4-dioxane (60 mL), and water (5 mL) was degassed with nitrogen, then the resulting mixture was heated at 60° C. for two days. After cooled to room temperature (RT), the mixture was concentrated, then diluted with water, and extracted with dichloromethane (DCM, 3×30 mL). The combined organic layers were dried over MgSO₄, filtered, and concentrated. The residue was purified by flash chromatography on a silica gel column with 8% ethyl acetate (EtOAc) in dichloromethane to afford the desired product. LCMS calculated for C₁₁H₈ClN₄ (M+H)⁺: 231.0. Found: 231.0.

### Step 2: 3-(5-Amino-2-(hydroxy(phenyl)methyl)-[1,2,4]triazolo[1,5-c]pyrimidin-7-yl)benzonitrile

A solution of 3-(2-amino-6-chloropyrimidin-4-yl)benzonitrile (100 mg, 0.434 mmol) and 2-hydroxy-2-phenylacetohydrazide (108 mg, 0.650 mmol) in ethanol (2 ml) was heated and stirred at 95° C. for 3 h. After cooling to RT, the reaction mixture was concentrated to dryness, taken into N,O-bis(trimethylsilyl)acetamide (1 mL) and stirred at 120° C. for 7 h. The resulting mixture was cooled to RT, poured onto ice, and stirred for 1 h. The resulting suspension was extracted with DCM three times. The combined organic layers were dried over MgSO₄, filtered, and concentrated. The residue was dissolved in methanol (MeOH) and purified by preparative LC-MS (pH 2, acetonitrile/water with TFA) to afford the product as TFA salt. LCMS calculated for C₁₉H₁₅N₆O (M+H)⁺: 343.1; found 343.1.

### Example A6: Synthesis of 3-(5-Amino-2-((2,6-difluorophenyl)(hydroxy)methyl)-[1,2,4]triazolo[1,5-c]pyrimidin-7yl)-2-fluorobenzonitrile

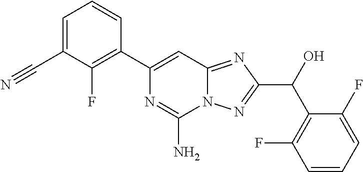 [SMILES: N#Cc1cccc(-c2cc3nc(C(O)c4c(F)cccc4F)nn3c(N)n2)c1F]

### Step 1: 3-(2-Amino-6-chloropyrimidin-4-yl)-2-fluorobenzonitrile

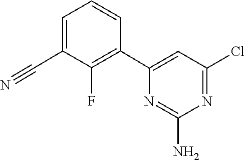 [SMILES: N#Cc1cccc(-c2cc(Cl)nc(N)n2)c1F]

To a solution of 3-bromo-2-fluorobenzonitrile (18.3 g, 91 mmol) in THF (60 mL) cooled to 0° C. was added i-PrMgCl LiCl complex (70.4 mL, 91 mmol) in THE (1.3 M) over 20 min. The mixture was stirred at 0° C. for 50 min, then zinc chloride (48.1 mL, 91 mmol) in 2-MeTHF (1.9 M) was added at 0° C. The reaction was stirred at r.t. for 25 min, at which point 4,6-dichloropyrimidin-2-amine (10 g, 61.0 mmol) was added in one portion. The solution was stirred for 10 min. Tetrakis(triphenylphosphine)palladium (1.41 g, 1.22 mmol) was added to the mixture and the reaction was stirred at r.t. for 16 h. Upon completion, 2,4,6-trimercaptotriazine silica gel (2 g) was added to the reaction solution. The mixture was stirred for 1 h and filtered. The solid was washed with ethyl acetate until the desired product had eluted completely (as detected by LCMS). The filtrate was washed with saturated ammonium chloride solution and water. The organics were concentrated to afford the crude product. Water was added to the crude material and the resulting precipitate was collected by filtration and dried under a stream of nitrogen. The crude material was taken forward without additional purification. LC-MS calculated for C₁₁H₇ClFN₄ (M+H)⁺: m/z=249.0; found 249.0.

### Step 2: Methyl 2-(2,6-difluorophenyl)-2-hydroxyacetate

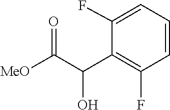 [SMILES: COC(=O)C(O)c1c(F)cccc1F]

Concentrated sulfuric acid (1.4 mL, 27 mmol) was added to a methanol (45 mL) solution of 2,6-difluoromandelic acid (5.0 g, 27 mmol) at 0° C. The mixture was stirred at r.t. for 4 h before being concentrated. To the resulting slurry was added saturated NaHCO₃ solution. The resulting mixture was extracted with DCM. The combined organic layers were washed with water, dried over MgSO₄, filtered, and concentrated to afford the crude product, which was used in the next step without further purification. LC-MS calculated for C₁₁H₁₂F₂NO₃ (M+H+MeCN)⁺: m/z=244.1; found 244.2.

### Step 3: 2-(2,6-Difluorophenyl)-2-hydroxyacetohydrazide

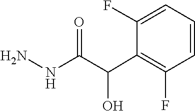 [SMILES: NNC(=O)C(O)c1c(F)cccc1F]

Hydrazine (3.0 mL, 96 mmol) was added to an ethanol (90 mL) solution of methyl 2-(2,6-difluorophenyl)-2-hydroxyacetate (10.8 g, 53 mmol) at RT. The reaction mixture was stirred at 100° C. for 2 h, cooled to RT, concentrated, and used in next step without further purification. LC-MS calculated for C₈H₉F2N₂O₂ (M+H)⁺: 203.1; found 203.2.

### Step 4: 3-(5-Amino-2-((2,6-difluorophenyl)(hydroxy)methyl)-[1,2,4]triazolo[1,5-c]pyrimidin-7-yl)-2-fluorobenzonitrile

The title compound was prepared using similar procedures as described for Example A5 Step 2, with 3-(2-amino-6-chloropyrimidin-4-yl)-2-fluorobenzonitrile replacing 3-(2-amino-6-chloropyrimidin-4-yl)benzonitrile, and with 2-(2,6-Difluorophenyl)-2-hydroxyacetohydrazide replacing 2-hydroxy-2-phenylacetohydrazide. The two enantiomers were separated by chiral SFC using a Phenomenex (R,R)-Whelk-01 column (21.2×250 mm, 5 m particle size) eluting with an isocratic mobile phase 15% MeOH in CO₂ with a flow rate of 85 mL/minute. The retention times of peak one and peak two were 3.8 min and 5.3 min, respectively. Following concentration, peak two was purified by prep-LCMS (pH=2, MeCN/water with TFA) to give the desired product as a TFA salt. LC-MS calculated for C₁₉H₁₂F₃N₆O (M+H)⁺: 397.1; found 397.1.

### Example A7: Synthesis of 5-Amino-7-(3-cyano-2-fluorophenyl)-2-((2,6-difluorophenyl)(hydroxy)methyl)-[1,2,4]triazolo[1,5-c]pyrimidine-8-carbonitrile

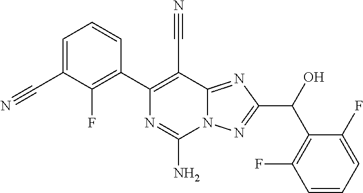 [SMILES: N#Cc1cccc(-c2nc(N)n3nc(C(O)c4c(F)cccc4F)nc3c2C#N)c1F]

### Step 1: 3-(5-Amino-8-bromo-2-((2,6-difluorophenyl)(hydroxy)methyl)-[1,2,4]triazolo[1,5-c]pyrimidin-7-yl)-2-fluorobenzonitrile

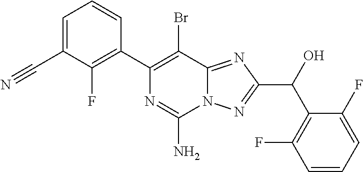 [SMILES: N#Cc1cccc(-c2nc(N)n3nc(C(O)c4c(F)cccc4F)nc3c2Br)c1F]

This compound was prepared using similar procedures as described for Example A1, Step 4, with 3-(5-amino-2-((2,6-difluorophenyl)(hydroxy)methyl)-[1,2,4]triazolo[1,5-c]pyrimidin-7-yl)-2-fluorobenzonitrile (from Example A6) replacing 3-(5-amino-2-(pyridin-2-ylmethyl)-[1,2,4]triazolo[1,5-c]pyrimidin-7-yl)benzonitrile. LCMS calculated for C₁₉H₁₁BrF₃N₆O (M+H)⁺: 475.0; found 475.0.

### Step 2: 5-Amino-7-(3-cyano-2-fluorophenyl)-2-((2,6-difluorophenyl)(hydroxy)methyl)-[1,2,4]triazolo[1,5-c]pyrimidine-8-carbonitrile

A mixture of 3-(5-amino-8-bromo-2-((2,6-difluorophenyl)(hydroxy)methyl)-[1,2,4]triazolo[1,5-c]pyrimidin-7-yl)-2-fluorobenzonitrile (0.12 g, 0.25 mmol), ZnCN₂ (0.060 g, 0.51 mmol) and tBuXPhos Pd G3 (0.020 g, 0.025 mmol) in 1,4-dioxane (0.63 mL) and water (0.63 mL) was purged with N2 and was stirred at 100° C. for 1 h. After cooling to r.t., the reaction was diluted with saturated NaHCO₃ and the organics were extracted with EtOAc (3×). The combined organics were dried over MgSO₄ and concentrated. The two enantiomers were separated by chiral HPLC using a Phenomenex Lux Celluose-4 column (21.2×250 mm, 5 m particle size) eluting with an isocratic mobile phase 60% EtOH in hexanes with a flow rate of 20 mL/minute. The retention times of peak one and peak two were 4.9 min and 7.2 min, respectively. Following concentration, peak one was purified by preparative LC-MS (pH 2, acetonitrile/water with TFA) to give the desired product as a TFA salt. LC-MS calculated for C₂₀H₁₁F₃N₇O (M+H)⁺: 422.1; found 422.1.

### Example A8: Synthesis of 3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile

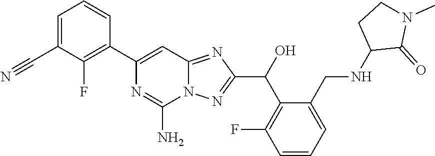 [SMILES: CN1CCC(NCc2cccc(F)c2C(O)c2nc3cc(-c4cccc(C#N)c4F)nc(N)n3n2)C1=O]

### Step 1: Methyl 2-(2-fluoro-6-vinylphenyl)acetate

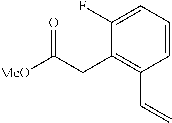

A mixture of methyl 2-(2-bromo-6-fluorophenyl)acetate (6.0 g, 24 mmol), potassium phosphate, tribasic (15.5 g, 73 mmol), palladium(II) acetate (0.55 g, 2.4 mmol), and SPhos (1.0 g, 2.4 mmol) were added to a 500 mL pressure vessel. Next, 4,4,5,5-tetramethyl-2-vinyl-1,3,2-dioxaborolane (6.4 ml, 36 mmol) in dioxane (150 mL) and water (15 mL) was added, the reaction mixture was purged with N2, and stirred at 80° C. for 16 h. The reaction mixture was then cooled to RT, concentrated, and extracted with EtOAc (×3). The combined organic layers were dried over MgSO₄, concentrated, and purified by column chromatography (0 to 50% EtOAc in DCM). LC-MS calculated for C₁₁H₁₂FO₂ (M+H)+: 195.1; found 195.1.

### Step 2: Methyl 2-(2-fluoro-6-vinylphenyl)-2-hydroxyacetate

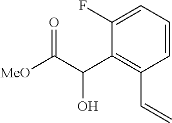

Methyl 2-(2-fluoro-6-vinylphenyl)acetate (2.5 g, 12.9 mmol) was dissolved in THF (130 mL) and cooled to −78° C. LDA (16.7 mL, 16.7 mmol) in THE (1.0 M) was added dropwise, and the resulting solution was stirred at −78° C. for 30 min. Then, 9,9-dimethyltetrahydro-4H-4a,7-methanobenzo[c][1,2]oxazireno[2,3-b]isothiazole 3,3-dioxide (4.7 g, 20.6 mmol) was added dropwise in THF (0.5 M). After 30 min at −78° C., the reaction mixture was warmed to 0° C. and stirred for 1 h. The reaction was quenched with saturated NH₄Cl. The aqueous layer was extracted with DCM (3×). The combined organics were dried over anhydrous Na₂SO₄, filtered, and concentrated under reduced pressure. The crude product was purified by column chromatography eluting with 0 to 50% ethyl acetate in hexanes to afford the desired product. LCMS calculated for C₁₁H₁₁FO₃Na (M+Na)⁺: 233.1; found 233.1.

### Step 3: 2-(2-Fluoro-6-vinylphenyl)-2-hydroxyacetohydrazide

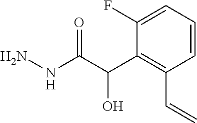

This compound was prepared using similar procedures as described for Example A6, Step 3, with methyl 2-(2-fluoro-6-vinylphenyl)-2-hydroxyacetate replacing methyl 2-(2,6-difluorophenyl)-2-hydroxyacetate. LCMS calculated for C₁₀H₁₂FN₂O₂ (M+H)⁺: 211.1; found 211.1.

### Step 4: 3-(5-Amino-2-((2-fluoro-6-vinylphenyl)(hydroxy)methyl)-[1,2,4]triazolo[1,5-c]pyrimidin-7-yl)-2-fluorobenzonitrile

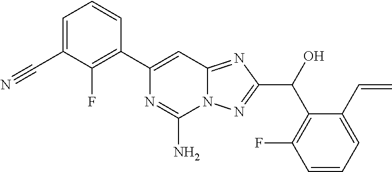 [SMILES: C=Cc1cccc(F)c1C(O)c1nc2cc(-c3cccc(C#N)c3F)nc(N)n2n1]

This compound was prepared using similar procedures as described for Example A6 Step 4, with 2-(2-fluoro-6-vinylphenyl)-2-hydroxyacetohydrazide replacing 2-(2,6-difluorophenyl)-2-hydroxyacetohydrazide. LCMS calculated for C₂₁H₁₅F2N60 (M+H)⁺: 405.1; found 405.1.

### Step 5: 3-(5-Amino-2-((2-fluoro-6-formylphenyl)(hydroxy)methyl)-[1,2,4]triazolo[1,5-c]pyrimidin-7-yl)-2-fluorobenzonitrile

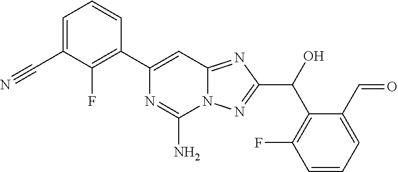 [SMILES: N#Cc1cccc(-c2cc3nc(C(O)c4c(F)cccc4C=O)nn3c(N)n2)c1F]

Osmium tetroxide in water (4% w/w, 0.36 mL, 0.12 mmol) was added to a THF (18 mL) and water (4.6 mL) solution of 3-(5-amino-2-((2-fluoro-6-vinylphenyl)(hydroxy)methyl)-[1,2,4]triazolo[1,5-c]pyrimidin-7-yl)-2-fluorobenzonitrile (930 mg, 2.30 mmol). The reaction mixture was stirred for 5 min at RT and then sodium periodate (2.5 g, 11.5 mmol) was added. After stirring for 1 h, the mixture was diluted with sodium metabisulfite in saturated aq. NaHCO₃ (5% w/w, 20 mL) and extracted with EtOAc (x3). The combined organic layers were dried over MgSO₄ and concentrated under reduced pressure. The crude material was purified by column chromatography eluting with 0 to 100% ethyl acetate in hexanes to afford the desired product. LCMS calculated for C₂₀H₁₃F₂N₆O₂ (M+H)⁺: 407.1; found 407.1.

### Step 6: 3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile

A solution of 3-amino-1-methylpyrrolidin-2-one (63 mg, 0.55 mmol) and 3-(5-amino-2-((2-fluoro-6-formylphenyl)(hydroxy)methyl)-[1,2,4]triazolo[1,5-c]pyrimidin-7-yl)-2-fluorobenzonitrile (150 mg, 0.37 mmol) was stirred at 40° C. for 2 h in 1,2-dichloroethane (1.9 mL). Then sodium triacetoxyborohydride (160 mg, 0.74 mmol) was added and the reaction mixture was stirred at room temperature for 16 h. The reaction was diluted with saturated NaHCO₃ and the organics were extracted with EtOAc (3×). The combined organics were dried over MgSO₄ and concentrated. The diastereomers were separated by chiral HPLC using a Phenomenex Lux Celluose-4 column (21.2×250 mm, 5 μm particle size) eluting with an isocratic mobile phase 45% EtOH in hexanes with a flow rate of 20 mL/minute. The retention times of peak one and peak two were 14.9 min and 17.5 min, respectively. Following concentration, peak two was further separated by chiral HPLC using a Phenomenex Lux Celluose-1 column (21.2×250 mm, 5 μm particle size) eluting with an isocratic mobile phase 30% EtOH in hexanes with a flow rate of 20 mL/minute. The retention times of peak one and peak two were 11.0 min and 15.5 min, respectively. Following concentration, peak one was purified by preparative LC-MS (pH=2, MeCN/water with TFA) to give the desired product as a TFA salt. LC-MS calculated for C₂₅H₂₃F₂N₈O₂ (M+H)⁺: 505.2; found 505.2.

### Example A9: Synthesis of 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile

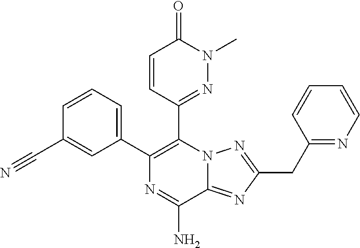 [SMILES: Cn1nc(-c2c(-c3cccc(C#N)c3)nc(N)c3nc(Cc4ccccn4)nn23)ccc1=O]

### Step 1: Methyl 3-bromo-1-(2-(3-cyanophenyl)-2-oxoethyl)-1H-1,2,4-triazole-5-carboxylate

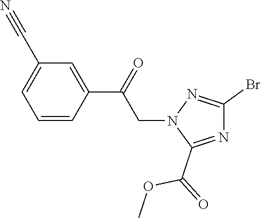 [SMILES: COC(=O)c1nc(Br)nn1CC(=O)c1cccc(C#N)c1]

To a solution of methyl 3-bromo-1H-1,2,4-triazole-5-carboxylate (5.0 g, 24.3 mmol), 3-(2-bromoacetyl)benzonitrile (5.44 g, 24.3 mmol) in DMF (100 mL) was added potassium carbonate (3.35 g, 24.3 mmol). The reaction mixture was stirred at ambient temperature for 2 h. The reaction mixture was then diluted with water and DCM. The organic layer was separated, washed with brine, dried over Na₂SO₄, filtered and concentrated. The resulting residue was purified via flash chromatography to give the desired product as a white solid (5.2 g, 61%). LC-MS calculated for C₁₃H₁₀BrN₄O₃ (M+H)⁺: m/z=349.0; found 349.0.

### Step 2: 3-(2-Bromo-8-oxo-7,8-dihydro-[1,2,4]triazolo[1,5-a]pyrazin-6-yl)benzonitrile

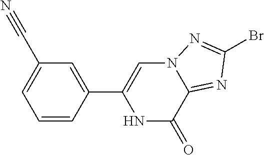 [SMILES: N#Cc1cccc(-c2cn3nc(Br)nc3c(=O)[nH]2)c1]

Methyl 3-bromo-1-(2-(3-cyanophenyl)-2-oxoethyl)-1H-1,2,4-triazole-5-carboxylate (10.5 g, 30.1 mmol) was dissolved in acetic acid (100 mL), and ammonium acetate (23.18 g, 301 mmol) was added. The mixture was stirred at 110° C. for 12 h. After cooling to room temperature, the reaction mixture was diluted with water. The resulting precipitate was collected via filtration, washed with water, and dried under vacuum to afford the product (8.4 g, 88%). LC-MS calculated for C₁₂H₇BrN₅O (M+H)⁺: m/z=316.0; found 316.0.

### Step 3: 3-(2-Bromo-8-chloro-[1,2,4]triazolo[1,5-a]pyrazin-6-yl)benzonitrile

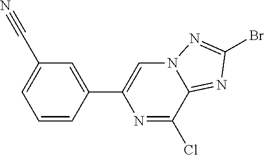 [SMILES: N#Cc1cccc(-c2cn3nc(Br)nc3c(Cl)n2)c1]

A mixture of 3-(2-bromo-8-oxo-7,8-dihydro-[1,2,4]triazolo[1,5-a]pyrazin-6-yl)benzonitrile (8.4 g, 26.6 mmol) and POCl₃ (49.5 mL, 531 mmol) was stirred at 110° C. overnight. After cooling to room temperature, the reaction mixture was slowly added to a flask containing ice and sodium bicarbonate. The resulting precipitate was collected, washed with water, and dried to afford the product (8.8 g, 99%). LC-MS calculated for C₁₂H₆BrClN₅ (M+H)⁺: m/z=333.9; found 334.0.

### Step 4. 3-(8-(Bis(4-methoxybenzyl)amino)-2-bromo-[1,2,4]triazolo[1,5-a]pyrazin-6-yl)benzonitrile

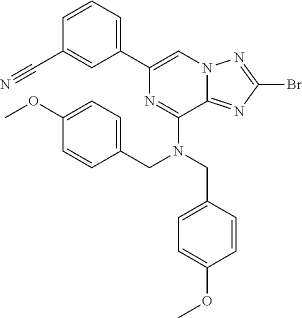 [SMILES: COc1ccc(CN(Cc2ccc(OC)cc2)c2nc(-c3cccc(C#N)c3)cn3nc(Br)nc23)cc1]

A mixture of 3-(2-bromo-8-chloro-[1,2,4]triazolo[1,5-a]pyrazin-6-yl)benzonitrile (8.99 g, 26.9 mmol), bis(4-methoxybenzyl)amine (10.37 g, 40.3 mmol), and DIPEA (9.4 mL, 53.7 mmol) in DMF (134 mL) was stirred at 85° C. overnight. The reaction mixture was cooled to room temperature, and diluted with water. The resulting precipitate was 15 collected via filtration, and dried to afford the product (14.1 g, 94%). LC-MS calculated for C₂₈H₂₄BrN₆O₂ (M+H)⁺: m/z=555.1; found 555.1.

### Step 5: 3-(8-(Bis(4-methoxybenzyl)amino)-2-(pyridin-2-ylmethyl)-[1,2,4]triazolo[1,5-a]pyrazin-6-yl)benzonitrile

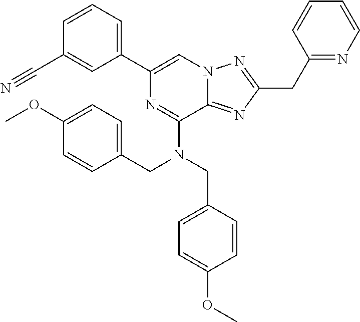 [SMILES: COc1ccc(CN(Cc2ccc(OC)cc2)c2nc(-c3cccc(C#N)c3)cn3nc(Cc4ccccn4)nc23)cc1]

To a solution of 2-methylpyridine (0.050 g, 0.540 mmol) in THE (0.5 mL) was added 2.5 M n-butyllithium (0.216 mL, 0.540 mmol) at −78° C. The resulting solution was stirred at the same temperature for 1 h, before 1.9 M zinc chloride in 2-methyltetrahydrofuran (0.284 mL, 0.540 mmol) was added, and the resulting mixture was stirred at room temperature for 10 min.

A microwave vial charge with 3-(8-(bis(4-methoxybenzyl)amino)-2-bromo-[1,2,4]triazolo[1,5-a]pyrazin-6-yl)benzonitrile (0.15 g, 0.270 mmol), palladium acetate (1.1 mg, 4.7 μmol), and 2′-(dicyclohexylphosphino)-N,N,N′,N′-tetramethylbiphenyl-2,6-diamine (4.1 mg, 9.5 μmol) was evacuated under high vacuum and backfilled with nitrogen. THE (2.0 mL) and toluene (0.5 mL) were then added to the reaction vial. The mixture was cooled to 0° C., and the zinc reagent prepared from previous step was added slowly via a syringe. The reaction mixture was then stirred at 60° C. overnight, cooled to room temperature, and partitioned between ethylacetate and saturated NH₄C₁ solution.

The layers were separated and the aqueous layer was extracted with ethylacetate. The combined organic layers were washed with water and brine, dried over MgSO₄, and concentrated. The resulting residue was purified via flash chromatography to afford the product (0.11 g, 71%). LC-MS calculated for C₃₄H₃₀N₇O₂ (M+H)⁺: m/z=568.2; found 568.3.

### Step 6. 3-(8-Amino-2-(pyridin-2-ylmethyl)-[1,2,4]triazolo[1,5-a]pyrazin-6-yl)benzonitrile

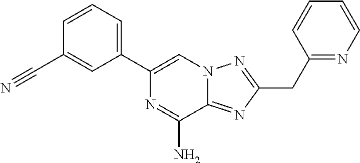 [SMILES: N#Cc1cccc(-c2cn3nc(Cc4ccccn4)nc3c(N)n2)c1]

A mixture of 3-(8-(bis(4-methoxybenzyl)amino)-2-(pyridin-2-ylmethyl)-[1,2,4]triazolo[1,5-a]pyrazin-6-yl)benzonitrile (110 mg, 0.194 mmol) and TFA (746 μL, 9.69 mmol) was stirred at 80° C. for 30 min, cooled to room temperature, and concentrated. The resulting residue was purified via prep-LCMS (pH 2) to give the product as a white solid (TFA salt) (57 mg, 90%). LC-MS calculated for C₁₈H₁₄N₇ (M+H)⁺: m/z=328.1; found 328.1.

### Step 7. 3-(8-Amino-5-bromo-2-(pyridin-2-ylmethyl)-[1,2,4]triazolo[1,5-a]pyrazin-6-yl)benzonitrile

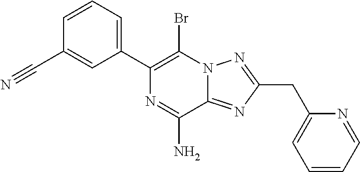 [SMILES: N#Cc1cccc(-c2nc(N)c3nc(Cc4ccccn4)nn3c2Br)c1]

To a solution of 3-(8-amino-2-(pyridin-2-ylmethyl)-[1,2,4]triazolo[1,5-a]pyrazin-6-yl)benzonitrile (TFA salt) (35 mg, 0.079 mmol) in DMF (0.5 mL)/DCM (0.5 mL) was added NBS (14.1 mg, 0.079 mmol). The reaction mixture was then stirred at room temperature for 1 h, and concentrated to afford the crude product, which was used in the next step without further purification. LC-MS calculated for C₁₈H₁₃BrN₇ (M+H)⁺: m/z=406.0; found 406.0.

### Step 8. 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile

A mixture of 6-chloro-2-methylpyridazin-3(2H)-one (30 mg, 0.21 mmol), bis(pinacolato)diboron (53 mg, 0.21 mmol), chloro(2-dicyclohexylphosphino-2′,4′,6′-triisopropyl-1,1′-biphenyl)[2-(2′-amino-1,1′-biphenyl)]palladium(II) (15.7 mg, 0.02 mmol) (XPhos Pd G2) and potassium acetate (61.7 mg, 0.63 mmol) in 1,4-dioxane (1 mL) was stirred at 100° C. for 1 h. 3-(8-Amino-5-bromo-2-(pyridin-2-ylmethyl)-[1,2,4]triazolo[1,5-a]pyrazin-6-yl)benzonitrile (10 mg, 0.025 mmol), cesium carbonate (37.6 mg, 0.116 mmol) and water (0.2 mL) were then added to the reaction mixture. The resulting mixture was heated at 90° C. for 1h. The mixture was concentrated and purified by preparative LCMS (pH 2, acetonitrile/water with TFA) to afford the desired product as TFA salt. LCMS calculated for C₂₃H₁₈N₉O (M+H)⁺: 436.2; found 436.2.

¹H NMR (500 MHz, DMSO) δ 8.66-8.62 (d, J=5.1 Hz, 1H), 8.09-8.02 (d, J=1.8 Hz, 1H), 7.88-7.85 (t, J=1.8 Hz, 1H), 7.85-7.81 (m, 3H), 7.78-7.72 (d, J=9.6 Hz, 1H), 7.66-7.51 (m, 4H), 7.10-7.06 (d, J=9.6 Hz, 1H), 4.59-4.48 (s, 2H), 3.53-3.43 (s, 3H).

### Example A10: Synthesis of 3-(8-Amino-2-((2,6-difluorophenyl)(hydroxy)methyl)-5-(pyrimidin-4-yl)-[1,2,4]triazolo[1,5-a]pyrazin-6-yl)benzonitrile

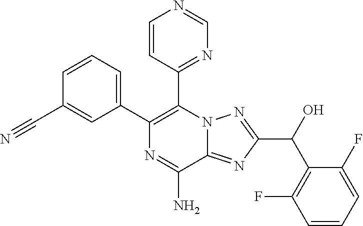 [SMILES: N#Cc1cccc(-c2nc(N)c3nc(C(O)c4c(F)cccc4F)nn3c2-c2ccncn2)c1]

### Step 1: Methyl 3-bromo-1-(2-(3-cyanophenyl)-2-oxoethyl)-1H-1,2,4-triazole-5-carboxylate

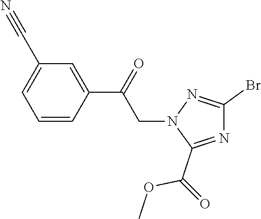 [SMILES: COC(=O)c1nc(Br)nn1CC(=O)c1cccc(C#N)c1]

To a solution of methyl 3-bromo-1H-1,2,4-triazole-5-carboxylate (5.0 g, 24.3 mmol), 3-(2-bromoacetyl)benzonitrile (5.44 g, 24.3 mmol) in DMF (100 mL) was added potassium carbonate (3.35 g, 24.3 mmol). The reaction mixture was stirred at ambient temperature for 2 h. The reaction mixture was then diluted with water and DCM. The organic layer was separated, washed with brine, dried over Na₂SO₄, filtered and concentrated. The resulting residue was purified via flash chromatography to give the desired product as a white solid (5.2 g, 61%). LC-MS calculated for C₁₃H₁₀BrN₄O₃ (M+H)⁺: m/z=349.0; found 349.0.

### Step 2: 3-(2-Bromo-8-oxo-7,8-dihydro-[1,2,4]triazolo[1,5-a]pyrazin-6-yl)benzonitrile

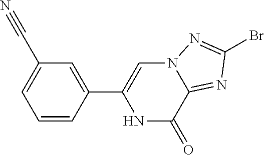 [SMILES: N#Cc1cccc(-c2cn3nc(Br)nc3c(=O)[nH]2)c1]

Methyl 3-bromo-1-(2-(3-cyanophenyl)-2-oxoethyl)-1H-1,2,4-triazole-5-carboxylate (10.5 g, 30.1 mmol) was dissolved in acetic acid (100 mL), and ammonium acetate (23.18 g, 301 mmol) was added. The mixture was stirred at 110° C. for 12 h. After cooling to room temperature, the reaction mixture was diluted with water. The resulting precipitate was collected via filtration, washed with water, and dried under vacuum to afford the product (8.4 g, 88%). LC-MS calculated for C₁₂H₇BrN₅O (M+H)⁺: m/z=316.0; found 316.0.

### Step 3: 3-(2-Bromo-8-chloro-[1,2,4]triazolo[1,5-a]pyrazin-6-yl)benzonitrile

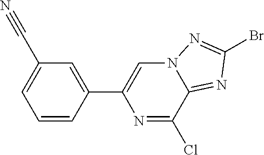 [SMILES: N#Cc1cccc(-c2cn3nc(Br)nc3c(Cl)n2)c1]

A mixture of 3-(2-bromo-8-oxo-7,8-dihydro-[1,2,4]triazolo[1,5-a]pyrazin-6-yl)benzonitrile (8.4 g, 26.6 mmol) and POCl₃ (49.5 mL, 531 mmol) was stirred at 110° C. overnight. After cooling to room temperature, the reaction mixture was slowly added to a flask containing ice and sodium bicarbonate. The resulting precipitate was collected via filtration, washed with water, and dried to afford the product (8.8 g, 99%). LC-MS calculated for C₁₂H₆BrClN₅ (M+H)⁺: m/z=336.0; found 336.0.

### Step 4: 3-(8-(Bis(4-methoxybenzyl)amino)-2-bromo-[1,2,4]triazolo[1,5-a]pyrazin-6-yl)benzonitrile

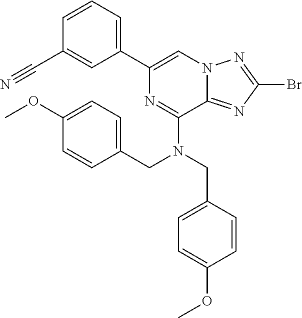 [SMILES: COc1ccc(CN(Cc2ccc(OC)cc2)c2nc(-c3cccc(C#N)c3)cn3nc(Br)nc23)cc1]

A mixture of 3-(2-bromo-8-chloro-[1,2,4]triazolo[1,5-a]pyrazin-6-yl)benzonitrile (8.99 g, 26.9 mmol), bis(4-methoxybenzyl)amine (10.37 g, 40.3 mmol), and DIPEA (9.4 mL, 53.7 mmol) in DMF (134 mL) was stirred at 65° C. overnight. The reaction mixture was cooled to room temperature, and diluted with water. The resulting precipitate was collected via filtration, and dried to afford the product (14.1 g, 94%). LC-MS calculated for C₂₈H₂₄BrN₆O₂ (M+H)⁺: m/z=555.1; found 555.1.

### STEP 5: 3-(8-(Bis(4-methoxybenzyl)amino)-2-vinyl-[1,2,4]triazolo[1,5-a]pyrazin-6-yl)benzonitrile

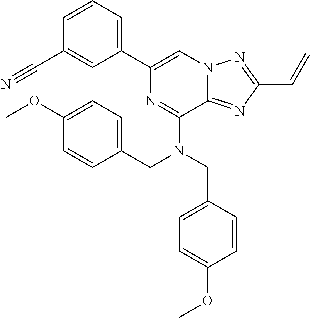 [SMILES: C=Cc1nc2c(N(Cc3ccc(OC)cc3)Cc3ccc(OC)cc3)nc(-c3cccc(C#N)c3)cn2n1]

A mixture of 3-(8-(bis(4-methoxybenzyl)amino)-2-bromo-[1,2,4]triazolo[1,5-a]pyrazin-6-yl)benzonitrile (10.0 g, 18.0 mmol), 4,4,5,5-tetramethyl-2-vinyl-1,3,2-dioxaborolane (3.88 g, 25.2 mmol), potassium phosphate tribasic (9.55 g, 45.0 mmol) and chloro(2-dicyclohexylphosphino-2′,4′,6′-triisopropyl-1,1′-biphenyl)[2-(2′-amino-1,1′-biphenyl)]palladium(II) (567 mg, 0.72 mmol) in 1,4-dioxane (200 mL) and water (50 mL) was stirred at 85° C. for 2 hrs. The reaction mixture was cooled to room temperature, and most of 1,4-dioxane was removed. The resulting precipitate was collected via filtration, washed with water and dried to afford the crude product (9.1 g), which was used in the next step directly. LC-MS calculated for C₃₀H₂₇N₆O₂ (M+H)⁺: m/z=503.2; found 503.1.

### Step 6. 3-(8-(Bis(4-methoxybenzyl)amino)-5-bromo-2-vinyl-[1,2,4]triazolo[1,5-a]pyrazin-6-yl)benzonitrile

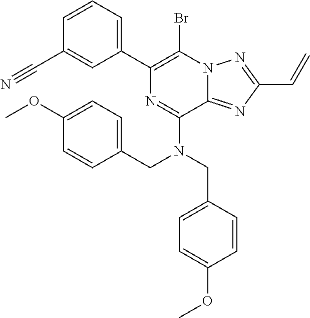 [SMILES: C=Cc1nc2c(N(Cc3ccc(OC)cc3)Cc3ccc(OC)cc3)nc(-c3cccc(C#N)c3)c(Br)n2n1]

To a solution of 3-(8-(bis(4-methoxybenzyl)amino)-2-vinyl-[1,2,4]triazolo[1,5-a]pyrazin-6-yl)benzonitrile (717 mg, 1.43 mmol) in 10 mL of dichloromethane, 1-bromopyrrolidine-2,5-dione (254 mg, 1.43 mmol) was added at 0° C. The resulting mixture was stirred for 4 hrs, and directly purified by a silica gel column to afford the desired product (780 mg, 94%). LC-MS calculated for C₃₀H26BrN₆O₂ (M+H)⁺: m/z=581.1; found 581.2.

### Step 7: 3-(8-(Bis(4-methoxybenzyl)amino)-5-(pyrimidin-4-yl)-2-vinyl-[1,2,4]triazolo[1,5-a]pyrazin-6-yl)benzonitrile

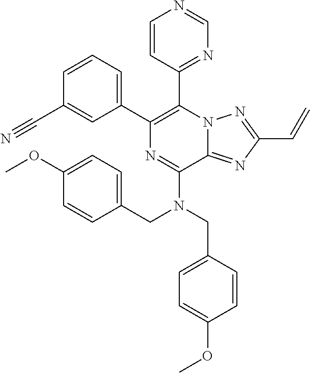 [SMILES: C=Cc1nc2c(N(Cc3ccc(OC)cc3)Cc3ccc(OC)cc3)nc(-c3cccc(C#N)c3)c(-c3ccncn3)n2n1]

A mixture of 3-(8-(bis(4-methoxybenzyl)amino)-5-bromo-2-vinyl-[1,2,4]triazolo[1,5-a]pyrazin-6-yl)benzonitrile (260 mg, 0.45 mmol), 4-(tributylstannyl)pyrimidine (215 mg, 0.58 mmol), lithium chloride (28.4 mg, 0.67 mmol), copper(I) chloride (67 mg, 0.67 mmol), and tetrakis(triphenylphosphine)palladium(0) (52 mg, 0.045 mmol) in THE (5 mL) was stirred at 90° C. for 45 mins. The reaction mixture was quenched with water and extracted with dichloromethane. The combined organic layers were concentrated and purified by a silica gel column to afford the desired product (176 mg, 67%). LC-MS calculated for C₃₄H₂₉N₈O₂ (M+H)⁺: m/z=581.2; found 581.1.

### Step 8: 3-(8-(Bis(4-methoxybenzyl)amino)-2-formyl-5-(pyrimidin-4-yl)-[1,2,4]triazolo[1,5-a]pyrazin-6-yl)benzonitrile

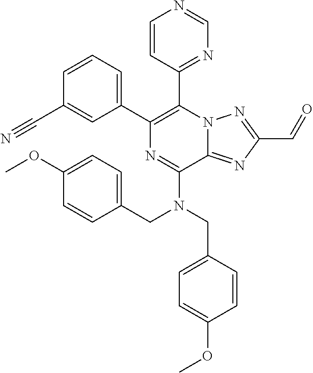 [SMILES: COc1ccc(CN(Cc2ccc(OC)cc2)c2nc(-c3cccc(C#N)c3)c(-c3ccncn3)n3nc(C=O)nc23)cc1]

A mixture of 3-(8-(bis(4-methoxybenzyl)amino)-5-(pyrimidin-4-yl)-2-vinyl-[1,2,4]triazolo[1,5-a]pyrazin-6-yl)benzonitrile (176 mg, 0.3 mmol), osmium(VIII) oxide (3 mg in 0.3 mL water, 0.015 mmol), and sodium periodate (292 mg, 1.36 mmol) in THF/water (1:1, 6 mL) was stirred at 65° C. for 1 h. The reaction mixture was cooled to room temperature, and extracted with dichloromethane. The combined organic layers were concentrated, and purified by silica gel column to afford the desired product (130 mg, 74%). LC-MS calculated for C₃₃H₂₇N₈O₃ (M+H)⁺: m/z=583.2; found 583.2.

### Step 9: 3-(8-Amino-2-((2,6-difluorophenyl)(hydroxy)methyl)-5-(pyrimidin-4-yl)-[1,2,4]triazolo[1,5-a]pyrazin-6-yl)benzonitrile

Preparation of the Grignard reagent: To a solution of 1,3-difluoro-2-iodobenzene (142 mg, 0.6 mmol) in tetrahydrofuran (1 mL), isopropylmagnesium chloride solution (296 μl, 2 M) was added at −10° C. The resulting mixture was stirred for 1 h, and used directly in the following step.

To a solution of 3-(8-(bis(4-methoxybenzyl)amino)-2-formyl-5-(pyrimidin-4-yl)-[1,2,4]triazolo[1,5-a]pyrazin-6-yl)benzonitrile (120 mg, 0.2 mmol) in THE (2 mL), the freshly prepared Grignard reagent from previous step was added at −10° C. The reaction mixture was stirred for 30 min, quenched with ammonium chloride solution (4 mL), and extracted with dichloromethane. The combined organic layers were concentrated under vacuum. The resulting material was dissolved in TFA (5 mL), and stirred at 80° C. for 20 min. The reaction mixture was then cooled to room temperature, concentrated, and basified by adding aqueous NaHCO₃ solution.

The crude material was directly purified by a silica gel column to afford the desired product (60 mg, 64%) as a racemic mixture. The product was then separated with chiral HPLC using a chiral column (Phenomenex Lux 5 um Cellulose-4, 21.2×250 mm) and 75% EtOH in hexanes (20 mL/min) solvent system.

Peak 2 was isolated, and further purified via preparative LC/MS (pH=2, acetonitrile/water with TFA) to give the desired product as a TFA salt. LC-MS calculated for C₂₃H₁₅F₂N₈O (M+H)⁺: m/z=457.1; found 457.0.

¹H NMR (600 MHz, DMSO-d₆) δ 9.14 (d, J=1.3 Hz, 1H), 8.95 (d, J=5.2 Hz, 1H), 7.90 (dd, J=5.2, 1.4 Hz, 1H), 7.88 (s, 1H), 7.78 (dt, J=7.6, 1.4 Hz, 1H), 7.74 (t, J=1.4 Hz, 1H), 7.54 (dt, J=7.9, 1.3 Hz, 1H), 7.51-7.40 (m, 2H), 7.09 (t, J=8.4 Hz, 2H), 6.27 (s, 1H).

### Example A11: Synthesis of 3-(8-amino-2-(amino(2,6-difluorophenyl)methyl)-5-(4-methyloxazol-5-yl)-[1,2,4]triazolo[1,5-a]pyrazin-6-yl)benzonitrile

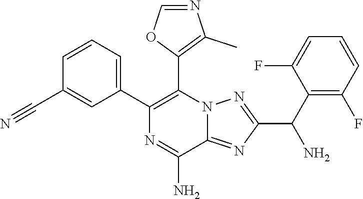 [SMILES: Cc1ncoc1-c1c(-c2cccc(C#N)c2)nc(N)c2nc(C(N)c3c(F)cccc3F)nn12]

### Step 1: 3-(8-(Bis(4-methoxybenzyl)amino)-5-bromo-2-vinyl-[1,2,4]triazolo[1,5-a]pyrazin-6-yl)benzonitrile

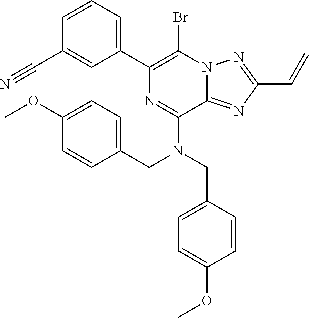 [SMILES: C=Cc1nc2c(N(Cc3ccc(OC)cc3)Cc3ccc(OC)cc3)nc(-c3cccc(C#N)c3)c(Br)n2n1]

To a solution of 3-(8-(bis(4-methoxybenzyl)amino)-2-vinyl-[1,2,4]triazolo[1,5-a]pyrazin-6-yl)benzonitrile (Example A10, Step 5; 241 mg, 0.48 mmol) in DCM (5 mL) was added NBS (84.6 mg, 0.48 mmol). The reaction mixture was then stirred at room temperature for 1 h, and concentrated to afford the crude product, which was used in the next step without further purification. LC-MS calculated for C₃₀H₂₆BrN₆O₂ (M+H)⁺: m/z=581.1; found 581.1.

### Step 2: 3-(8-(bis(4-methoxybenzyl)amino)-5-bromo-2-formyl-[1,2,4]triazolo[1,5-a]pyrazin-6-yl)benzonitrile

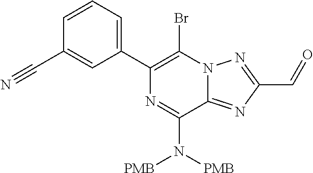

A mixture of 3-(8-(bis(4-methoxybenzyl)amino)-5-bromo-2-vinyl-[1,2,4]triazolo[1,5-a]pyrazin-6-yl)benzonitrile (174 mg, 0.3 mmol), osmium(VIII) oxide (3 mg in 0.3 mL water, 0.015 mmol), and sodium periodate (292 mg, 1.36 mmol) in THF/water (1:1, 6 mL) was stirred at 65° C. for 1 h. The reaction mixture was cooled to room temperature, and extracted with dichloromethane. The combined organic layers were concentrated, and purified by silica gel column to afford the desired product. LC-MS calculated for C₂₉H₂₄N₆O₃Br (M+H)⁺: m/z=583.1; found 583.1.

### Step 3: 3-(8-(bis(4-methoxybenzyl)amino)-5-bromo-2-((2,6-difluorophenyl)(hydroxy)methyl)-[1,2,4]triazolo[1,5-a]pyrazin-6-yl)benzonitrile

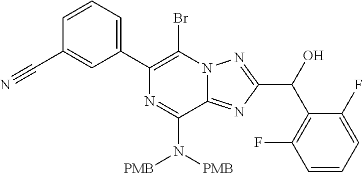

Preparation of the Grignard reagent: To a solution of 1,3-difluoro-2-iodobenzene (142 mg, 0.6 mmol) in tetrahydrofuran (1 mL), isopropylmagnesium chloride solution (296 μl, 2 M) was added at −10° C. The resulting mixture was stirred for 1 h, and used directly in the following step.

To a solution of 3-(8-(bis(4-methoxybenzyl)amino)-5-bromo-2-formyl-[1,2,4]triazolo[1,5-a]pyrazin-6-yl)benzonitrile (120 mg, 0.2 mmol) in THE (2 mL), the freshly prepared Grignard reagent from previous step was added at −10° C. The reaction mixture was stirred for 30 min, quenched with ammonium chloride solution (4 mL), and extracted with dichloromethane. The combined organic layers were concentrated under vacuum and purified by a silica gel column to afford the desired product as a racemic mixture. LC-MS calculated for C₃₅H₂₈N₆O₃BrF₂ (M+H)⁺: m/z=697.1; found 697.1.

### Step 4: 3-(8-(bis(4-methoxybenzyl)amino)-2-((2,6-difluorophenyl)(hydroxy)methyl)-5-(4-methyloxazol-5-yl)-[1,2,4]triazolo[1,5-a]pyrazin-6-yl)benzonitrile

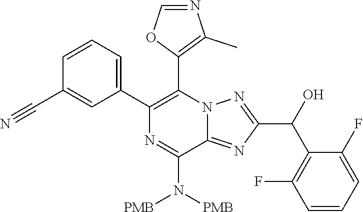

A mixture of 3-(8-(bis(4-methoxybenzyl)amino)-5-bromo-2-((2,6-difluorophenyl)(hydroxy)methyl)-[1,2,4]triazolo[1,5-a]pyrazin-6-yl)benzonitrile (382 mg, 0.55 mmol), 4-methyl-5-(4,4,5,5-tetramethyl-1,3,2-dioxaborolan-2-yl)oxazole (137 mg, 0.65 mmol), dicyclohexyl(2′,4′,6′-triisopropylbiphenyl-2-yl)phosphine-(2′-aminobiphenyl-2-yl)(chloro)palladium (1:1) (17 mg, 21.6 μmol) and Cs₂CO₃ (356 mg, 1.09 mmol) in 1,4-dioxane (2 mL) and water (200 μl) was purged with N₂ and heated at 95° C. for 7 h. The mixture was concentrated and purified via flash chromatography to afford the desired product as a colorless oil. LCMS calculated for C₃₉H₃₂N₇O₄F₂ (M+H)⁺: 700.2; found 700.2.

### Step 5: 3-(8-(bis(4-methoxybenzyl)amino)-2-(chloro(2,6-difluorophenyl)methyl)-5-(4-methyloxazol-5-yl)-[1,2,4]triazolo[1,5-a]pyrazin-6-yl)benzonitrile

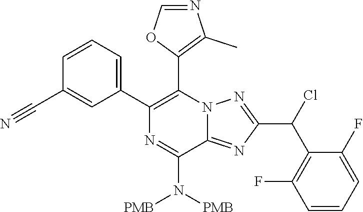

To a solution of 3-(8-(bis(4-methoxybenzyl)amino)-2-((2,6-difluorophenyl)(hydroxy)methyl)-5-(4-methyloxazol-5-yl)-[1,2,4]triazolo[1,5-a]pyrazin-6-yl)benzonitrile (201 mg, 0.29 mmol) in 2 mL of dichloromethane, thionyl chloride (105 μl, 1.435 mmol) was added at rt. The resulting mixture was stirred for 4h, concentrated and used in next step without any further purification. LC-MS calculated for C₃₉H₃₁N₇O₃ClF₂ (M+H)⁺: m/z=718.2; found 718.2.

### Step 6: 3-(8-amino-2-(amino(2,6-difluorophenyl)methyl)-5-(4-methyloxazol-5-yl)-[1,2,4]triazolo[1,5-a]pyrazin-6-yl)benzonitrile

To a solution of 3-(8-(bis(4-methoxybenzyl)amino)-2-(chloro(2,6-difluorophenyl)methyl)-5-(4-methyloxazol-5-yl)-[1,2,4]triazolo[1,5-a]pyrazin-6-yl)benzonitrile (40 mg, 0.084 mmol) in 1 mL of DMSO was added ammonia solution (1 mL). The mixture was heated with microwave condition at 100° C. for 10 h before diluted with water and extracted with EtOAc. The combined organic layers were washed with water and brine, dried over MgSO₄, and concentrated. The resulting residue was dissolved in TFA (1 mL), and stirred at 80° C. for 20 min. The reaction mixture was then cooled to room temperature, concentrated, and basified by adding aq. NaHCO₃ solution. The crude material was directly purified by a silica gel column to afford the desired product as a racemic mixture. The product was then separated with chiral HPLC using a chiral column (AM-1) and 45% EtOH in hexanes (20 mL/min) solvent system. Peak 1 was isolated, and further purified via preparative LC/MS (pH=2, acetonitrile/water with TFA) to give the desired product as a TFA salt. LC-MS calculated for C₂₃H₁₇F₂N₈O (M+H)⁺: m/z=459.1; found 459.0.

### Example A12: Synthesis of 3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile

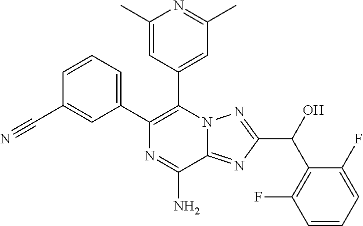 [SMILES: Cc1cc(-c2c(-c3cccc(C#N)c3)nc(N)c3nc(C(O)c4c(F)cccc4F)nn23)cc(C)n1]

To a solution of 3-(8-(bis(4-methoxybenzyl)amino)-5-bromo-2-((2,6-difluorophenyl)(hydroxy)methyl)-[1,2,4]triazolo[1,5-a]pyrazin-6-yl)benzonitrile (Example A11, Step 3; 0.518 g, 0.638 mmol), 2,6-dimethyl-4-(4,4,5,5-tetramethyl-1,3,2-dioxaborolan-2-yl)pyridine (0.346 g, 1.48 mmol), and dicyclohexyl(2′,4′,6′-triisopropylbiphenyl-2-yl)phosphine-(2′-aminobiphenyl-2-yl)(chloro)palladium (1:1) (0.058 g, 0.074 mmol) in dioxane (3.0 mL) and water (0.60 mL) was added potassium phosphate tribasic (0.472 g, 2.23 mmol). The reaction mixture was stirred at 90° C. for 1 h. The reaction mixture was then diluted with water and DCM. The layers were separated, the aqueous layer was extracted with DCM, and the combined organic fractions were dried over MgSO₄, filtered and concentrated. The crude material was dissolved in TFA (5 mL) and heated to 80° C. for 20 minutes. The reaction mixture was then cooled to room temperature, concentrated, and basified by adding aqueous NaHCO₃ solution. The crude material was directly purified by a silica gel column to afford the desired product (257 mg, 72%) as a racemic mixture.

The product was then separated with chiral HPLC using a chiral column (Phenomenex Lux Sum Cellulose-2, 21.1×250 mm) and 35% EtOH in Hexanes (20 mL/min) solvent system. Peak 2 was isolated, and further purified using preparative LC-MS (pH=2, acetonitrile/water with TFA) to give the desired product as a TFA salt. LC-MS calculated for C₂₆H₂₀F₂N₇O (M+H)⁺: m/z=484.2; found 484.2. ¹H NMR (500 MHz, DMSO-d₆) δ 7.92 (s, 2H), 7.85 (s, 1H), 7.83 (d, J=7.6 Hz, 1H), 7.56 (d, J=8.0 Hz, 1H), 7.53-7.40 (m, 4H), 7.10 (t, J=8.4 Hz, 2H), 6.27 (s, 1H), 2.51 (s, 6H).

### Example A13: Synthesis of 3-(4-amino-2-(pyridin-2-ylmethyl)-7-(pyrimidin-4-yl)-2H-[1,2,3]triazolo[4,5-c]pyridin-6-yl)benzonitrile

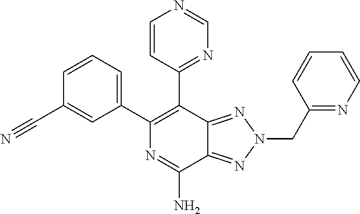

### Step 1. 4,6-dichloro-3H-[1,2,3]triazolo[4,5-c]pyridine

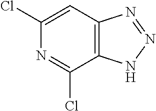 [SMILES: Clc1cc2nn[nH]c2c(Cl)n1]

A solution of NaNO₂ (3.88 g, 56.2 mmol) in water (3 mL) was added to a solution of 2,6-dichloropyridine-3,4-diamine (10 g, 56 mmol) in hydrochloric acid, 37% (5 mL) at 0° C. The solution was stirred for 30 min. Water (20 mL) was added and the white precipitate was filtered, washed with water, and dried to give the desired product. LC-MS calculated for C₅H₃C₁₂N₄: 189.0 (M+H)⁺; found: 189.0 (M+H)⁺.

### Step 2. 6-chloro-N-(2,4-dimethoxybenzyl)-3H-[1,2,3]triazolo[4,5-c]pyridin-4-amine

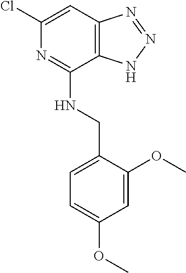 [SMILES: COc1ccc(CNc2nc(Cl)cc3nn[nH]c23)c(OC)c1]

The mixture of 4,6-dichloro-3H-[1,2,3]triazolo[4,5-c]pyridine (600 mg, 3.17 mmol), (2,4-dimethoxyphenyl)methanamine (0.53 mL, 3.49 mmol) and triethylamine (0.53 mL, 3.81 mmol) in 1,4-dioxane (10 mL) was stirred at 110° C. for 3 days. Direct purification on silica gel column afforded the desired product (875 mg, 86%). LC-MS calculated for C₁₄H₁₅C₁N₅O₂: 320.1 (M+H)⁺; found: 320.3 (M+H)⁺.

### Step 3. 6-chloro-N-(2,4-dimethoxybenzyl)-2-(pyridin-2-ylmethyl)-2H-[1,2,3]triazolo[4,5-c]pyridin-4-amine

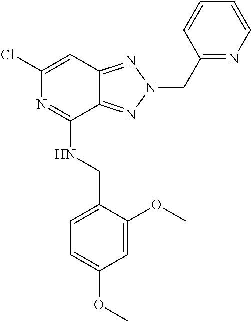 [SMILES: COc1ccc(CNc2nc(Cl)cc3nn(Cc4ccccn4)nc23)c(OC)c1]

The mixture of 6-chloro-N-(2,4-dimethoxybenzyl)-3H-[1,2,3]triazolo[4,5-c]pyridin-4-amine (875 mg, 2.74 mmol), pyridin-2-ylmethanol (0.317 mL, 3.28 mmol) and triphenylphosphine (1436 mg, 5.47 mmol) in DCM (20 mL) was added diisopropyl azodicarboxylate (0.647 mL, 3.28 mmol) at 0° C. The resulting mixture was stirred at 0° C. for 1 h. Direct purification on silica gel column afforded the desired product (375 mg, 33.4% yield). LC-MS calculated for C₂₀H₂₀ClN₆O₂: 411.1 (M+H)⁺; found: 411.2 (M+H)⁺.

### Step 4. 3-(4-((2,4-dimethoxybenzyl)amino)-2-(pyridin-2-ylmethyl)-2H-[1,2,3]triazolo[4,5-c]pyridin-6-yl)benzonitrile

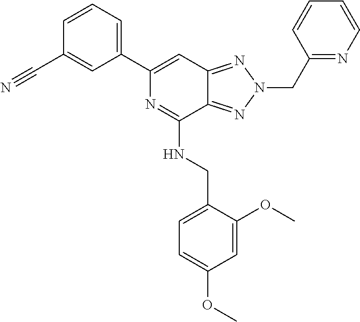 [SMILES: COc1ccc(CNc2nc(-c3cccc(C#N)c3)cc3nn(Cc4ccccn4)nc23)c(OC)c1]

To the mixture of 6-chloro-N-(2,4-dimethoxybenzyl)-2-(pyridin-2-ylmethyl)-2H-[1,2,3]triazolo[4,5-c]pyridin-4-amine (375 mg, 0.913 mmol) and (3-cyanophenyl)boronic acid (268 mg, 1.825 mmol) in 1,4-dioxane (10 mL) and water (1.00 mL) was added cesium carbonate (595 mg, 1.825 mmol). The resulting mixture was purged with N₂ and then chloro(2-dicyclohexylphosphino-2′,4′,6′-triisopropyl-1,1′-biphenyl)[2-(2′-amino-1,1′-biphenyl)]palladium(II) (71.8 mg, 0.091 mmol) was added. The reaction mixture was stirred at 120° C. under microwave irradiation for 90 min. The reaction was quenched with 20 mL of ethyl acetate and 20 mL of water. The organic phase was separated and the aqueous solution was extracted with ethyl acetate twice. The combined extracts were dried over Na₂SO₄, filtered and evaporated under reduced pressure. The residue was purified on silica gel column to afford the desired product (300 mg, 68.9%). LC-MS calculated for C₂₇H₂₄N₇O₂: 478.2 (M+H)⁺; found: 478.3 (M+H)⁺.

### Step 5. 3-(4-amino-2-(pyridin-2-ylmethyl)-2H-[1,2,3]triazolo[4,5-c]pyridin-6-yl)benzonitrile

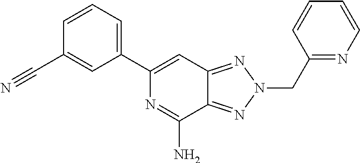

The solution of 3-(4-((2,4-dimethoxybenzyl)amino)-2-(pyridin-2-ylmethyl)-2H-[1,2,3]triazolo[4,5-c]pyridin-6-yl)benzonitrile (300.3 mg, 0.629 mmol) in TFA (5 mL) was stirred at 100° C. for 30 min. TFA was evaporated under reduced pressure and then 20 mL of saturated NaHCO₃ aqueous solution and 20 mL of ethyl acetate were added. The organic phase was separated and the aqueous solution was extracted with ethyl acetate twice. The combined extracts were dried over Na₂SO₄, filtered and evaporated under reduced pressure. The residue was purified on silica gel column to afford the desired product (175 mg, 85%). LC-MS calculated for C₁₈H₁₄N₇: 328.1 (M+H)⁺; found: 328.2 (M+H)⁺.

### Step 6. 3-(4-amino-7-bromo-2-(pyridin-2-ylmethyl)-2H-[1,2,3]triazolo[4,5-c]pyridin-6-yl)benzonitrile

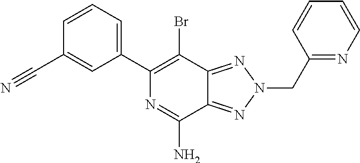

The mixture of 3-(4-amino-2-(pyridin-2-ylmethyl)-2H-[1,2,3]triazolo[4,5-c]pyridin-6-yl)benzonitrile (175 mg, 0.535 mmol) and 1-bromopyrrolidine-2,5-dione (100 mg, 0.561 mmol) in THE (10 mL) was stirred at 0° C. for 30 min and then quenched with saturated NaHCO₃ aqueous solution. The organic phase was separated, dried over Na₂SO₄, filtered and evaporated under reduced pressure. The resulting residue was purified on silica gel column to afford the desired product (135 mg, 62.2%). LC-MS calculated for C₁₈H₁₃BrN₇: 406.0 (M+H)⁺ and 408.0 (M+H)⁺; found: 406.1 (M+H)⁺ and 408.2 (M+H)⁺.

### Step 7. 3-(4-amino-2-(pyridin-2-ylmethyl)-7-(pyrimidin-4-yl)-2H-[1,2,3]triazolo[4,5-c]pyridin-6-yl)benzonitrile

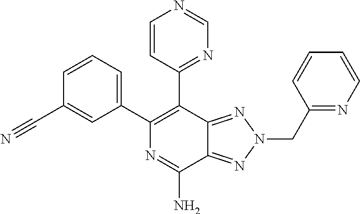

A mixture of 3-(4-amino-7-bromo-2-(pyridin-2-ylmethyl)-2H-[1,2,3]triazolo[4,5-c]pyridin-6-yl)benzonitrile (182 mg, 0.448 mmol), 4-(tributylstannyl)pyrimidine (496 mg, 1.344 mmol), and copper(I) chloride (53.2 mg, 0.538 mmol), lithium chloride (22.79 mg, 0.538 mmol) and tetrakis(triphenylphosphine)palladium(0) (51.8 mg, 0.045 mmol) in THF (1 mL) was first purged with N₂, and then heated and stirred at 90° C. for 2 h. The reaction was diluted with methanol and purified with prep-LCMS (pH=2) to give the desired product. LC-MS calculated for C₂₂H₁₆N₉: 406.2 (M+H)⁺; found: 406.2 (M+H)⁺.

### Example A14: Synthesis of 3-(4-amino-2-((3-fluoropyridin-2-yl)methyl)-7-(pyrimidin-4-yl)-2H-[1,2,3]triazolo[4,5-c]pyridin-6-yl)benzonitrile

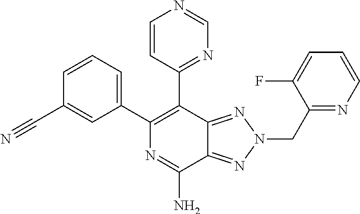

### Step 1. 6-chloro-N-(2,4-dimethoxybenzyl)-2-((3-fluoropyridin-2-yl)methyl)-2H-[1,2,3]triazolo[4,5-c]pyridin-4-amine

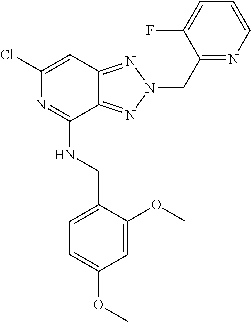 [SMILES: COc1ccc(CNc2nc(Cl)cc3nn(Cc4ncccc4F)nc23)c(OC)c1]

To the mixture of 6-chloro-N-(2,4-dimethoxybenzyl)-3H-[1,2,3]triazolo[4,5-c]pyridin-4-amine (Example A13, Step 2; 1000 mg, 3.13 mmol), (3-fluoropyridin-2-yl)methanol (477 mg, 3.75 mmol) and triphenylphosphine (1641 mg, 6.25 mmol) in DCM (1.7 mL) was added diisopropyl azodicarboxylate (739 μl, 3.75 mmol) at 0° C. The reaction mixture was stirred at 0° C. for 1 h. Direct purification on silica gel column afforded the desired product (433 mg, 32%). LC-MS calculated for C₂₀H₁₉ClFN₆O₂: 429.1 (M+H)⁺; found: 429.3 (M+H)⁺.

### Step 2. 3-(4-((2,4-dimethoxybenzyl)amino)-2-((3-fluoropyridin-2-yl)methyl)-2H-[1,2,3]triazolo[4,5-c]pyridin-6-yl)benzonitrile

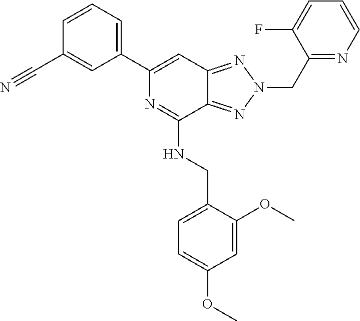 [SMILES: COc1ccc(CNc2nc(-c3cccc(C#N)c3)cc3nn(Cc4ncccc4F)nc23)c(OC)c1]

Cesium carbonate (658 mg, 2.019 mmol) was added to the mixture of 6-chloro-N-(2,4-dimethoxybenzyl)-2-((3-fluoropyridin-2-yl)methyl)-2H-[1,2,3]triazolo[4,5-c]pyridin-4-amine (433 mg, 1.010 mmol) and (3-cyanophenyl)boronic acid (297 mg, 2.019 mmol) in 1,4-dioxane (10.0 mL) and water (1.0 mL). The resulting mixture was sparged with N₂ for 2 min and (SP-4-4)-[2′-Amino[1,1′-biphenyl]-2-yl]chloro[dicyclohexyl[2′,4′,6′-tris(1-methylethyl)[1,1′-biphenyl]-2-yl]phosphine]palladium (79 mg, 0.101 mmol) was added. The reaction mixture was stirred at 120° C. for 1.5 h under microwave irradiation. The reaction was quenched with 20 mL of ethyl acetate and 20 mL of water. The organic phase was separated and the aqueous solution was extracted with ethyl acetate twice. The combined extracts were dried over Na₂SO₄, filtered and evaporated under reduced pressure. The residue was purified on silica gel column to afford the desired product (357 mg, 71%). LC-MS calculated for C₂₇H₂₃FN₇O₂: 496.2 (M+H)⁺; found: 496.3 (M+H)⁺.

### Step 3. 3-(4-amino-2-((3-fluoropyridin-2-yl)methyl)-2H-[1,2,3]triazolo[4,5-c]pyridin-6-yl)benzonitrile

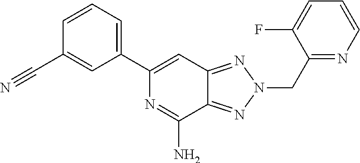 [SMILES: N#Cc1cccc(-c2cc3nn(Cc4ncccc4F)nc3c(N)n2)c1]

The solution of 3-(4-((2,4-dimethoxybenzyl)amino)-2-((3-fluoropyridin-2-yl)methyl)-2H-[1,2,3]triazolo[4,5-c]pyridin-6-yl)benzonitrile (357.3 mg, 0.721 mmol) in TFA (5 mL) was stirred at 100° C. for 1 h. TFA was evaporated under reduced pressure and then 20 mL of saturated NaHCO₃ aqueous solution and 20 mL of ethyl acetate were added. The organic phase was separated and the aqueous solution was extracted with ethyl acetate twice. The combined extracts were dried over Na₂SO₄, filtered and evaporated under reduced pressure. The residue was purified on silica gel column to afford the desired product (213 mg, 61%). LC-MS m/z calculated for C₁₈H₁₃FN₇: 346.1 (M+H)⁺; found: 346.3 (M+H)⁺.

### Step 4. 3-(4-amino-7-bromo-2-((3-fluoropyridin-2-yl)methyl)-2H-[1,2,3]triazolo[4,5-c]pyridin-6-yl)benzonitrile

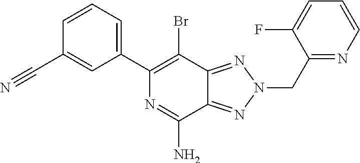 [SMILES: N#Cc1cccc(-c2nc(N)c3nn(Cc4ncccc4F)nc3c2Br)c1]

The mixture of 3-(4-amino-2-((3-fluoropyridin-2-yl)methyl)-2H-[1,2,3]triazolo[4,5-c]pyridin-6-yl)benzonitrile (213 mg, 0.617 mmol) and 1-bromopyrrolidine-2,5-dione (220 mg, 1.234 mmol) in THE (5 mL) was stirred at 0° C. for 1 h. Direct purification on silica gel afforded the desired product (175 mg, 67%). LC-MS calculated for C₁₈H₁₂BrFN₇: 424.0 (M+H)⁺ and 426.0 (M+H)⁺; found: 424.3 (M+H)⁺ and 426.3 (M+H)⁺.

### Step 5. 3-(4-amino-2-((3-fluoropyridin-2-yl)methyl)-7-(pyrimidin-4-yl)-2H-[1,2,3]triazolo[4,5-c]pyridin-6-yl)benzonitrile

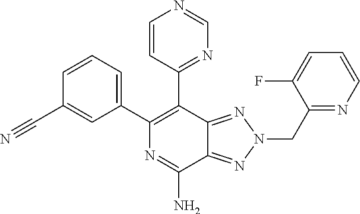 [SMILES: N#Cc1cccc(-c2nc(N)c3nn(Cc4ncccc4F)nc3c2-c2ccncn2)c1]

The mixture of 3-(4-amino-7-bromo-2-((3-fluoropyridin-2-yl)methyl)-2H-[1,2,3]triazolo[4,5-c]pyridin-6-yl)benzonitrile (220 mg, 0.519 mmol), 4-(tributylstannyl)pyrimidine (383 mg, 1.037 mmol), and copper(I) chloride (61.6 mg, 0.622 mmol), lithium chloride (26.4 mg, 0.622 mmol) and tetrakis(triphenylphosphine)palladium(0) (59.9 mg, 0.052 mmol) in THE (1 mL) was first purged with N₂, and then heated and stirred at 90° C. for 2 h. The reaction was diluted with methanol and purified with prep-LCMS (pH=2) to give the desired product. LC-MS calculated for C₂₂H₅FN₉: 424.1 (M+H)⁺; found: 424.3 (M+H)⁺. ¹H NMR (500 MHz, DMSO-d₆) ppm 8.98 (s, 1H), 8.77 (d, J=5.02 Hz, 1H), 8.38 (dd, J₁=4.60 Hz, J₂=1.32 Hz, 1H), 7.90-8.30 (bs, 2H), 7.76-7.89 (m, 3H), 7.66 (dd, J₁=5.25 Hz, J₂=1.25 Hz, 1H), 7.45-7.58 (m, 3H), 6.25 (s, 2H).

### Example A15: Synthesis of 3-(4-amino-2-((3-fluoropyridin-2-yl)methyl)-7-(pyridin-4-yl)-2H-[1,2,3]triazolo[4,5-c]pyridin-6-yl)benzonitrile

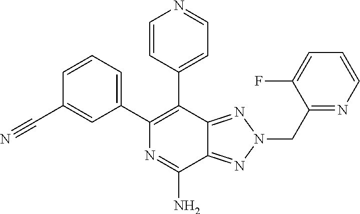 [SMILES: N#Cc1cccc(-c2nc(N)c3nn(Cc4ncccc4F)nc3c2-c2ccncc2)c1]

Cesium carbonate (46.1 mg, 0.141 mmol) was added to a mixture of 3-(4-amino-7-bromo-2-((3-fluoropyridin-2-yl)methyl)-2H-[1,2,3]triazolo[4,5-c]pyridin-6-yl)benzonitrile (30 mg, 0.071 mmol) and pyridin-4-ylboronic acid (17.38 mg, 0.141 mmol) in 1,4-dioxane (2 mL) and water (0.2 mL). The resulting mixture was sparged with N₂ for 2 min and chloro(2-dicyclohexylphosphino-2′,4′,6′-triisopropyl-1,1′-biphenyl)[2-(2′-amino-1,1′-biphenyl)]palladium(II) (5.56 mg, 7.07 μmol) was added. The reaction mixture was stirred at 120° C. for 1.5 h under microwave irradiation. The reaction mixture was diluted with methanol. Direct purification on prep. HPLC afforded the desired product. LC-MS calculated for C₂₃H₁₆FN₈: 423.1 (M+H)⁺; found: 423.3 (M+H)⁺.

### Example A16: Synthesis of 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile

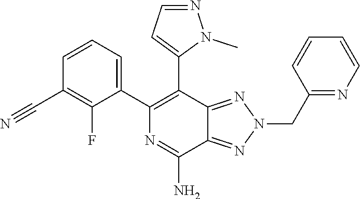 [SMILES: Cn1nccc1-c1c(-c2cccc(C#N)c2F)nc(N)c2nn(Cc3ccccn3)nc12]

### Step 1. 3-(4-amino-7-bromo-2-(pyridin-2-ylmethyl)-2H-[1,2, 3]triazolo[4,5-c]pyridin-6-yl)-2-fluorobenzonitrile

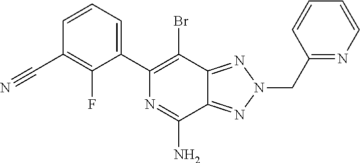 [SMILES: N#Cc1cccc(-c2nc(N)c3nn(Cc4ccccn4)nc3c2Br)c1F]

This compound was prepared by following a similar procedure from Example A13, Step 1 to Step 6, with (3-cyano-2-fluorophenyl)boronic acid replacing (3-cyanophenyl)boronic acid in Step 4. LC-MS calculated for C₁₈H₁₂BrFN₇: 424.0 (M+H)⁺ and 426.0 (M+H)⁺; found: 424.3 (M+H)⁺ and 426.3 (M+H)⁺.

### Step 2. 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile

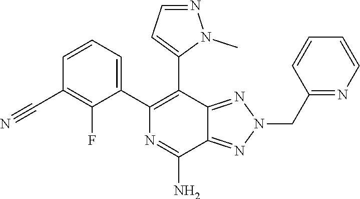 [SMILES: Cn1nccc1-c1c(-c2cccc(C#N)c2F)nc(N)c2nn(Cc3ccccn3)nc12]

This compound was prepared by following a similar procedure in Example A15, with (1-methyl-1H-pyrazol-5-yl)boronic acid replacing pyridin-4-ylboronic acid, and with 3-(4-amino-7-bromo-2-(pyridin-2-ylmethyl)-2H-[1,2,3]triazolo[4,5-c]pyridin-6-yl)-2-fluorobenzonitrile replacing 3-(4-amino-7-bromo-2-((3-fluoropyridin-2-yl)methyl)-2H-[1,2,3]triazolo[4,5-c]pyridin-6-yl)benzonitrile. LC-MS calculated for C₂₂H₁₇FN₉: 426.2 (M+H)⁺; found: 426.3 (M+H)⁺.

### Example A17: Synthesis of 7-(1-((5-Chloropyridin-3-yl)methyl)-1H-pyrazol-4-yl)-3-methyl-9-pentyl-6,9-dihydro-5H-pyrrolo[3,2-d][1,2,4]triazolo[4,3-a]pyrimidin-5-one

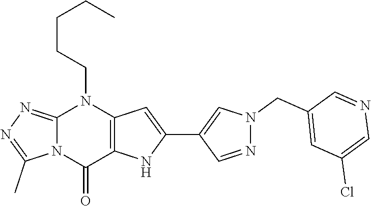 [SMILES: CCCCCn1c2cc(-c3cnn(Cc4cncc(Cl)c4)c3)[nH]c2c(=O)n2c(C)nnc12]

### Step 1: Ethyl 3-(pentylamino)-1H-pyrrole-2-carboxylate

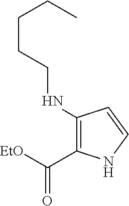 [SMILES: CCCCCNc1cc[nH]c1C(=O)OCC]

Ethyl 3-amino-1H-pyrrole-2-carboxylate (5 g, 32.4 mmol), pentanal (3.79 ml, 35.7 mmol), and sodium cyanoborohydride (2.038 g, 32.4 mmol) were mixed in methanol (64.9 ml) at room temperature overnight. The reaction mixture was concentrated under reduced pressure. The crude residue was purified by flash chromatography (0 to 100% EtOAc in hexanes) to give the desired product (4.4 g, 61%). LCMS calculated for C₁₂H₂₁N₂O₂ (M+H): 225.2. Found: 225.1.

### Step 2: Ethyl 3-(3-(ethoxycarbonyl)-1-pentylthioureido)-1H-pyrrole-2-carboxylate

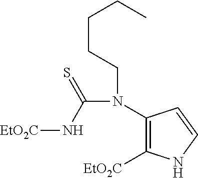 [SMILES: CCCCCN(C(=S)NC(=O)OCC)c1cc[nH]c1C(=O)OCC]

A vial was charged with ethyl 3-(pentylamino)-1H-pyrrole-2-carboxylate (4.4 g, 19.62 mmol), dichloromethane (39.2 ml), and ethoxycarbonyl isothiocyanate (2.78 ml, 23.54 mmol). The reaction mixture was stirred at room temperature overnight. The reaction mixture was quenched with water (40 ml), and the layers were separated. The aqueous layer was extracted with dichloromethane (3×40 mL), and the combined organic fractions were dried over MgSO₄, filtered, and concentrated. The crude material was used in the next step without further purification (7.3 g, quant.). LCMS calculated for C₁₆H₂₆N₃O₄S (M+H): 356.2. Found: 356.1.

Step 3: 1-Pentyl-2-thioxo-2,3-dihydro-1H-pyrrolo[3,2-d]pyrimidin-4(5H)-one

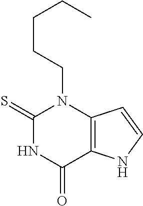 [SMILES: CCCCCn1c(=S)[nH]c(=O)c2[nH]ccc21]

A microwave vial was charged with ethyl 3-(3-(ethoxycarbonyl)-1-pentylthioureido)-1H-pyrrole-2-carboxylate (7.31 g, 20.57 mmol) and sodium ethoxide (21% w/w, 8.45 ml, 22.62 mmol) solution. The vial was capped and heated in a microwave reactor for 10 minutes at 120 degrees Celsius. The reaction mixture was brought to neutral pH on addition of 1M HCl solution, and the solid product was filtered and dried (3.1 g, 64%). LCMS calculated for C₁₁H₁₆N₃OS (M+H): 238.1. Found: 238.1.

### Step 4: 2-Hydrazono-1-pentyl-2,3-dihydro-1H-pyrrolo[3,2-d]pyrimidin-4(5H)-one

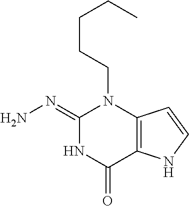 [SMILES: CCCCCn1/c(=N/N)[nH]c(=O)c2[nH]ccc21]

A vial was charged with 1-pentyl-2-thioxo-2,3-dihydro-1H-pyrrolo[3,2-d]pyrimidin-4(5H)-one (3.13 g, 13.19 mmol) and hydrazine hydrate (20 mL). The reaction mixture was stirred at 100 degrees Celsius overnight. The solid formed was filtered and washed with water to give the desired product (2.2 g, 70%). LCMS calculated for C₁₁H₁₈N₅O (M+H): 236.1. Found: 236.1.

### Step 5: 3-Methyl-9-pentyl-6,9-dihydro-5H-pyrrolo[3,2-d][1,2,4]triazolo[4,3-a]pyrimidin-5-one

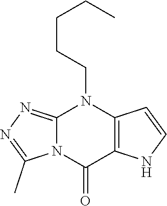 [SMILES: CCCCCn1c2cc[nH]c2c(=O)n2c(C)nnc12]

A vial was charged with (E)-2-hydrazono-1-pentyl-2,3-dihydro-1H-pyrrolo[3,2-d]pyrimidin-4(5H)-one (4.8 g, 20.40 mmol), a drop of trifluoroacetic acid, and triethyl orthoacetate (20 mL). The reaction mixture was heated to 110 degrees Celsius for three hours. The suspension was filtered, washed with hexanes, and dried (4.0 g, 76%). LCMS calculated for C₁₃H₁₈N₅O (M+H): 260.1. Found: 260.2.

### Step 6: 3-Methyl-9-pentyl-6-(phenylsulfonyl)-6,9-dihydro-5H-pyrrolo[3,2-d][1,2,4]triazolo[4,3-a]pyrimidin-5-one

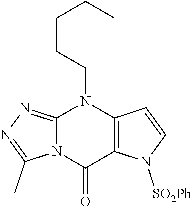

A vial was charged with 3-methyl-9-pentyl-6,9-dihydro-5H-pyrrolo[3,2-d][1,2,4]triazolo[4,3-a]pyrimidin-5-one (from Step 1) (4 g, 15.43 mmol), dichloromethane (40 mL), dimethylaminopyridine (0.188 g, 1.543 mmol), triethylamine (3.23 ml, 23.14 mmol), and benzenesulfonyl chloride (2.187 ml, 16.97 mmol). The reaction mixture was stirred at room temperature for one hour. The reaction mixture was quenched with water, and the layers were separated. The aqueous layer was extracted with dichloromethane (3×40 mL), and the combined organic fractions were dried over MgSO₄, filtered, and concentrated. The crude material was used in the next step without further purification (6.1 g, quant.). LCMS calculated for C₁₉H₂₂N₅O₃S (M+H): 400.1. Found: 400.1.

### Step 7: 7-Bromo-3-methyl-9-pentyl-6-(phenylsulfonyl)-6,9-dihydro-5H-pyrrolo[3,2-d][1,2,4]triazolo[4,3-a]pyrimidin-5-one

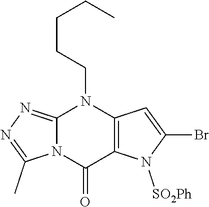

A vial was charged with 3-methyl-9-pentyl-6-(phenylsulfonyl)-6,9-dihydro-5H-pyrrolo[3,2-d][1,2,4]triazolo[4,3-a]pyrimidin-5-one (1 g, 2.503 mmol), dry THE (30 mL) and the mixture was cooled to −78 degrees Celsius. Lithium diisopropylamide solution (1M in hexanes/THF, 3.13 ml, 3.13 mmol) was added dropwise. The reaction mixture was maintained at −78° C. for 1.5 hours. A solution of 1,2-dibromo-1,1,2,2-tetrachloroethane (1.223 g, 3.75 mmol) in dry THE (3 ml) was added dropwise to the reaction mixture and the reaction mixture was maintained at −78° C. for a further 1.5 hours. The reaction mixture was quenched with sat. aq. NH₄C₁ solution (30 mL) and diluted with dichloromethane (30 mL). The layers were separated and the aqueous layer was extracted with DCM (3×30 mL). The combined organic fractions were dried over MgSO₄, filtered, and concentrated. The crude residue was purified by automated flash chromatography (0 to 100% EtOAc in DCM) to give the desired product (0.84 g, 70%). LCMS calculated for C₁₉H₂₁BrN₅O₃S (M+H): 478.1. Found: 478.1.

### Step 8: 3-Chloro-5-((4-(4,4,5,5-tetramethyl-1,3,2-dioxaborolan-2-yl)-1H-pyrazol-1-yl)methyl)pyridine

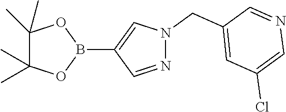 [SMILES: CC1(C)OB(c2cnn(Cc3cncc(Cl)c3)c2)OC1(C)C]

A vial was charged with 4-(4,4,5,5-tetramethyl-1,3,2-dioxaborolan-2-yl)-1H-pyrazole (0.5 g, 2.58 mmol), 3-(bromomethyl)-5-chloropyridine hydrobromide (0.741 g, 2.58 mmol), cesium carbonate (2.52 g, 7.73 mmol), and DMF (6.44 ml). The reaction mixture was stirred at 60 degrees Celsius for one hour. The reaction mixture was quenched with water (10 ml) and diluted with dichloromethane (10 ml). The layers were separated, and the aqueous layer was extracted with dichloromethane (3×10 mL). The combined dichloromethane extracts were dried over MgSO₄, filtered, and concentrated. Purification by automated flash chromatography (0 to 100% EtOAc in DCM) afforded the product (0.548 g, 67%). LCMS calculated for C₁₅H₂₀BClN₃O₂ (M+H): 320.1, 322.1. Found: 320.1, 322.1.

### Step 9: 7-(1-((5-Chloropyridin-3-yl)methyl)-1H-pyrazol-4-yl)-3-methyl-9-pentyl-6,9-dihydro-5H-pyrrolo[3,2-d][1,2,4]triazolo[4,3-a]pyrimidin-5-one

A vial was charged with 7-bromo-3-methyl-9-pentyl-6-(phenylsulfonyl)-6,9-dihydro-5H-pyrrolo[3,2-d][1,2,4]triazolo[4,3-a]pyrimidin-5-one (0.01 g, 0.021 mmol), 3-chloro-5-((4-(4,4,5,5-tetramethyl-1,3,2-dioxaborolan-2-yl)-1H-pyrazol-1-yl)methyl)pyridine (0.013 g, 0.042 mmol), chloro(2-dicyclohexylphosphino-2′,4′,6′-triisopropyl-1,1′-biphenyl)[2-(2′-amino-1,1′-biphenyl)]palladium(II) (5.00 mg, 0.006 mmol) and potassium phosphate tribasic (0.016 g, 0.074 mmol). 1,4-dioxane (0.35 ml) and water (0.07 ml) were added and the reaction mixture was sparged with nitrogen gas for 5 minutes then stirred at 90° C. for two hours. The reaction mixture was cooled to room temperature and sodium hydroxide (10 mg) was added. The reaction mixture was stirred at 40 degrees Celsius for 60 minutes. The reaction mixture was cooled to room temperature and diluted with DMF (5 ml). Purification by preparative HPLC (pH 2, acetonitrile/water with TFA) afforded the product as a TFA salt (2 mg, 21%). LCMS calculated for C₂₂H₂₄C₁N₈₀ (M+H): 451.2, 453.2. Found: 451.2, 453.2.

### Example A18: Synthesis of 3-Methyl-7-(1-((5-methylpyridin-3-yl)methyl)-1H-pyrazol-4-yl)-9-pentyl-6,9-dihydro-5H-pyrrolo[3,2-d][1,2,4]triazolo[4,3-a]pyrimidin-5-one

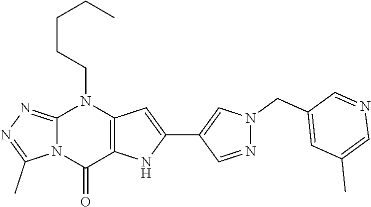 [SMILES: CCCCCn1c2cc(-c3cnn(Cc4cncc(C)c4)c3)[nH]c2c(=O)n2c(C)nnc12]

This compound was prepared using similar procedures as described in Example A17 using 3-(bromomethyl)-5-methylpyridine in place of 3-(bromomethyl)-5-chloropyridine hydrobromide in Step 8. LCMS calculated for C₂₃H₂₇N₈O (M+H): 431.2. Found: 431.3.

### Example A19: Synthesis of 3-Methyl-9-pentyl-7-(1-(thieno[3,2-b]pyridin-6-ylmethyl)-1H-pyrazol-4-yl)-6,9-dihydro-5H-pyrrolo[3,2-d][1,2,4]triazolo[4,3-a]pyrimidin-5-one

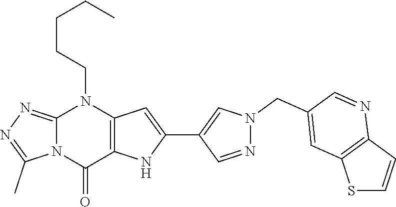 [SMILES: CCCCCn1c2cc(-c3cnn(Cc4cnc5ccsc5c4)c3)[nH]c2c(=O)n2c(C)nnc12]

This compound was prepared using similar procedures as described in Example A17 using 6-(bromomethyl)thieno[3,2-b]pyridine in place of 3-(bromomethyl)-5-chloropyridine hydrobromide in Step 8. LCMS calculated for C₂₄H₂₅N₈OS (M+H): 473.2. Found: 473.3.

### Example A20: 7-(1-((2-(2-(Dimethylamino)acetyl)-1,2,3,4-tetrahydroisoquinolin-6-yl)methyl)-1H-pyrazol-4-yl)-3-methyl-9-pentyl-6,9-dihydro-5H-pyrrolo[3,2-d][1,2,4]triazolo[4,3-a]pyrimidin-5-one

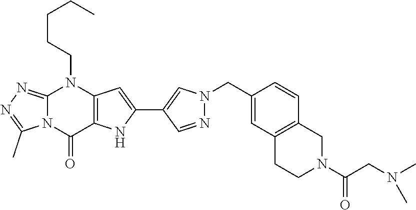 [SMILES: CCCCCn1c2cc(-c3cnn(Cc4ccc5c(c4)CCN(C(=O)CN(C)C)C5)c3)[nH]c2c(=O)n2c(C)nnc12]

### Step 1: tert-Butyl 6-((4-(4,4,5,5-tetramethyl-1,3,2-dioxaborolan-2-yl)-1H-pyrazol-1-yl)methyl)-3,4-dihydroisoquinoline-2(1H)-carboxylate

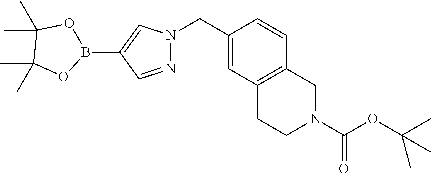 [SMILES: CC(C)(C)OC(=O)N1CCc2cc(Cn3cc(B4OC(C)(C)C(C)(C)O4)cn3)ccc2C1]

A flask was charged with 4-(4,4,5,5-tetramethyl-1,3,2-dioxaborolan-2-yl)-1H-pyrazole (0.5 g, 2.58 mmol), tert-butyl 6-(hydroxymethyl)-3,4-dihydroisoquinoline-2(1H)-carboxylate (0.339 g, 1.288 mmol), triphenylphosphine (0.743 g, 2.83 mmol), and THE (12 ml). The solution was cooled to 0° C. and DIAD (0.601 ml, 3.09 mmol) was added dropwise. The reaction mixture was stirred overnight at room temperature. The mixture was diluted with ethyl acetate and washed with water, dried and concentrated. The product was purified by column chromatography eluting with Hexane/EtOAc (max. EtOAc 60%) to afford the product. LCMS calculated for C₂₄H₃₅BN₃O₄ (M+H)⁺: m/z=440.3; found 440.3.

### Step 2: 7-bromo-3-methyl-9-pentyl-6,9-dihydro-5H-pyrrolo[3,2-d][1,2,4]triazolo[4,3-a]pyrimidin-5-one

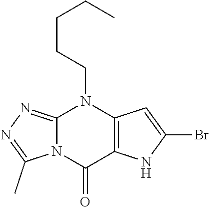 [SMILES: CCCCCn1c2cc(Br)[nH]c2c(=O)n2c(C)nnc12]

TBAF (1.0 M in THF) (2.0 ml, 2.0 mmol) was added to a solution of 7-bromo-3-methyl-9-pentyl-6-(phenylsulfonyl)-6,9-dihydro-5H-pyrrolo[3,2-d][1,2,4]triazolo[4,3-a]pyrimidin-5-one (0.360 g, 0.753 mmol) in THE (4.0 ml), and then the reaction was stirred at 50° C. for 1 h. The solvent was removed and the product was purified by column chromatography eluting with CH₂Cl₂/MeOH (max. MeOH 10%). LCMS calculated for C₁₃H₁₇BrN₅O (M+H)⁺: m/z=338.1; found 338.1.

### Step 3: tert-Butyl 6-((4-(3-methyl-5-oxo-9-pentyl-6,9-dihydro-5H-pyrrolo[3,2-d][1,2,4]triazolo[4,3-a]pyrimidin-7-yl)-H-pyrazol-1-yl)methyl)-3,4-dihydroisoquinoline-2(1H)-carboxylate

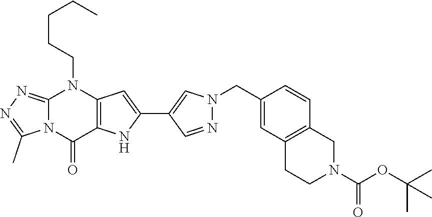 [SMILES: CCCCCn1c2cc(-c3cnn(Cc4ccc5c(c4)CCN(C(=O)OC(C)(C)C)C5)c3)[nH]c2c(=O)n2c(C)nnc12]

A mixture of 7-bromo-3-methyl-9-pentyl-6,9-dihydro-5H-pyrrolo[3,2-d][1,2,4]triazolo[4,3-a]pyrimidin-5-one (from Example A20, Step 2) (0.040 g, 0.118 mmol), tert-butyl 6-((4-(4,4,5,5-tetramethyl-1,3,2-dioxaborolan-2-yl)-1H-pyrazol-1-yl)methyl)-3,4-dihydroisoquinoline-2(1H)-carboxylate (0.062 g, 0.142 mmol), dichloro[1,1′-bis(dicyclohexylphosphino)ferrocene]palladium(II), dichloromethane adduct (Pd-127) (8.94 mg, 0.012 mmol) and cesium fluoride (0.090 g, 0.591 mmol) in t-BuOH (1.5 ml)/Water (0.6 ml) was vacuumed and replaced with N₂ for 3 times. The reaction was then stirred at 105° C. for 2 h, cooled to rt, diluted with ethyl acetate, washed with water, dried and concentrated. The product was purified by column eluting with CH₂Cl₂/MeOH (max. MeOH 10%). LCMS calculated for C₃₁H₃₉N₈O₃ (M+H)⁺: m/z=571.3; found 571.5.

### Step 4: 3-Methyl-9-pentyl-7-(1-((1,2,3,4-tetrahydroisoquinolin-6-yl)methyl)-1H-pyrazol-4-yl)-6,9-dihydro-5H-pyrrolo[3,2-d][1,2,4]triazolo[4,3-a]pyrimidin-5-one

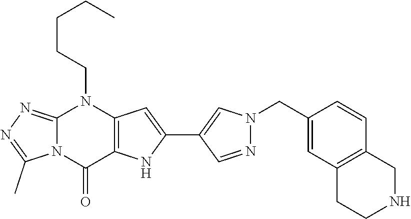 [SMILES: CCCCCn1c2cc(-c3cnn(Cc4ccc5c(c4)CCNC5)c3)[nH]c2c(=O)n2c(C)nnc12]

TFA (0.5 ml, 6.49 mmol) was added to a solution of tert-butyl 6-((4-(3-methyl-5-oxo-9-pentyl-6,9-dihydro-5H-pyrrolo[3,2-d][1,2,4]triazolo[4,3-a]pyrimidin-7-yl)-1H-pyrazol-1-yl)methyl)-3,4-dihydroisoquinoline-2(1H)-carboxylate (50.0 mg, 0.088 mmol) in CH₂Cl₂ (0.5 ml), and then the reaction was stirred at room temperature for 30 min. The solvent was then removed to provide the crude product as TFA salt. LCMS calculated for C₂₆H₃₁N₈O (M+H)⁺: m/z=471.3; found 471.2.

### Step 5: 7-(1-((2-(2-(Dimethylamino)acetyl)-1,2,3,4-tetrahydroisoquinolin-6-yl)methyl)-1H-pyrazol-4-yl)-3-methyl-9-pentyl-6,9-dihydro-5H-pyrrolo[3,2-d][1,2,4]triazolo[4,3-a]pyrimidin-5-one

Dimethylglycinoyl chloride (3.10 mg, 0.026 mmol) was added to a solution of 3-methyl-9-pentyl-7-(1-((1,2,3,4-tetrahydroisoquinolin-6-yl)methyl)-1H-pyrazol-4-yl)-6,9-dihydro-5H-pyrrolo[3,2-d][1,2,4]triazolo[4,3-a]pyrimidin-5-one (6.0 mg, 0.013 mmol) 15 and triethylamine (8.89 μl, 0.064 mmol) in CH₂C₁₂ (0.8 ml) at room temperature and stirred for 30 min. The solvent was removed, and the mixture was diluted with acetonitrile/water and purified by prep HPLC (pH 2, acetonitrile/water with TFA) to provide the desired compound as its TFA salt. LC-MS calculated for C₃₀H₃₈N₉O₂ (M+H)⁺: m/z=556.3; found 556.3.

### Example A21. 3-(2-((5-(1H-Pyrazol-1-yl)-2H-tetrazol-2-yl)methyl)-5-amino-8-(pyrimidin-4-yl)-[1,2,4]triazolo[1,5-c]pyrimidin-7-yl)benzonitrile (Compound 21A) and 3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile (Compound 21B)

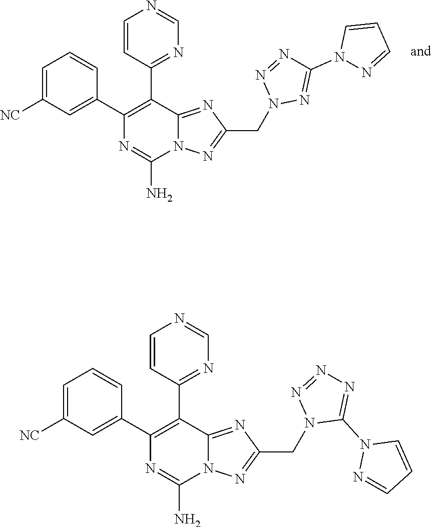

The mixture of title compounds was prepared using similar procedures as described for Example A3, with 5-(1H-pyrazol-1-yl)-1H-tetrazole replacing 2-(1H-tetrazol-5-yl)pyridine. Compound 21A was purified by preparative LC-MS (pH 2, acetonitrile/water with TFA) to afford the product as a TFA salt. LCMS calculated for C₂₁H₁₅N₁₄ (M+H)⁺: 463.2; found 463.2.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating cancer in a subject, comprising administering to the subject:
(i) an inhibitor of A2A/A2B;
(ii) an inhibitor of PD-1/PD-L1; and
(iii) an inhibitor of human CD73.

2. The method of claim 1, wherein the inhibitor of A2A/A2B is a compound of Formula (I): 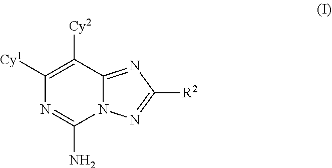
or a pharmaceutically acceptable salt thereof, wherein:
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₇ cycloalkyl-C₁₋₃ alkyl-, (5-7 membered heteroaryl)-C₁₋₃-alkyl-, and (4-7 membered heterocycloalkyl)-C₁₋₃ alkyl- of R² are each optionally substituted with 1, 2, or 3 independently selected R^{C} substituents;
R^{a2} is (5-7 membered heteroaryl)-C₁₋₃ alkyl- optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and
each R^{a4}, R^{c4}, and R^{d4} are independently selected from H and C₁₋₆ alkyl.

3. The method of claim 1, wherein the inhibitor of A2A/A2B is selected from:
3-(5-amino-2-(pyridin-2-ylmethyl)-8-(pyrimidin-4-yl)-[1,2,4]triazolo[1,5-c]pyrimidin-7-yl)benzonitrile;
3-(5-amino-2-((2,6-difluorophenyl)(hydroxy)methyl)-8-(pyrimidin-4-yl)-[1,2,4]triazolo[1,5-c]pyrimidin-7-yl)benzonitrile;
3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile;
3-(5-amino-2-((3-methylpyridin-2-yl)methoxy)-8-(pyrimidin-4-yl)-[1,2,4]triazolo[1,5-c]pyrimidin-7-yl)benzonitrile; and
3-(2-((5-(1H-pyrazol-1-yl)-2H-tetrazol-2-yl)methyl)-5-amino-8-(pyrimidin-4-yl)-[1,2,4]triazolo[1,5-c]pyrimidin-7-yl)benzonitrile;
or a pharmaceutically acceptable salt of any of the aforementioned.

4. The method of claim 1, wherein the inhibitor of A2A/A2B is a compound of Formula (II): 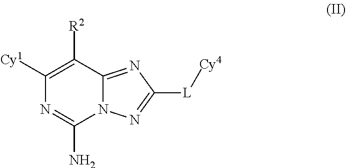
or a pharmaceutically acceptable salt thereof, wherein:
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected RD substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₆ alkyl, 5-6 membered heteroaryl, and 4-7 membered heterocycloalkyl of R^{8A} are each optionally substituted with 1, 2, or 3 independently selected R^{8B} substituents;
each R^{a81}, R_{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;
each R^{8B} is independently selected from halo and C₁₋₃ alkyl; and
each R^{8D} is independently selected from OH, CN, halo, C₁₋₆ alkyl, and C₁₋₆ haloalkyl.

5. The method of claim 1, wherein the inhibitor of A2A/A2B is selected from:
3-(5-amino-2-(hydroxy(phenyl)methyl)-[1,2,4]triazolo[1,5-c]pyrimidin-7-yl)benzonitrile;
3-(5-amino-2-((2,6-difluorophenyl)(hydroxy)methyl)-[1,2,4]triazolo[1,5-c]pyrimidin-7-yl)-2-fluorobenzonitrile;
5-amino-7-(3-cyano-2-fluorophenyl)-2-((2,6-difluorophenyl)(hydroxy)methyl)-[1,2,4]triazolo[1,5-c]pyrimidine-8-carbonitrile; and
3-(5-amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile;
or a pharmaceutically acceptable salt of any of the aforementioned.

6. The method of claim 1, wherein the inhibitor of A2A/A2B is a compound of Formula (III): 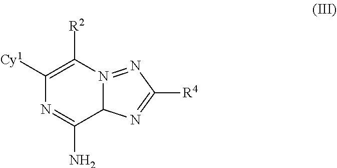
or a pharmaceutically acceptable salt thereof, wherein:
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41}, and R^{d41} is independently selected from H and C₁₋₆ alkyl.

7. The method of claim 1, wherein the inhibitor of A2A/A2B is selected from:
3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile;
3-(8-amino-2-((2,6-difluorophenyl)(hydroxy)methyl)-5-(pyrimidin-4-yl)-[1,2,4]triazolo[1,5-a]pyrazin-6-yl)benzonitrile;
3-(8-amino-2-(amino(2,6-difluorophenyl)methyl)-5-(4-methyloxazol-5-yl)-[1,2,4]triazolo[1,5-a]pyrazin-6-yl)benzonitrile; and
3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile;
or a pharmaceutically acceptable salt of any of the aforementioned.

8. The method of claim 1, wherein the inhibitor of A2A/A2B is a compound of Formula (IV): 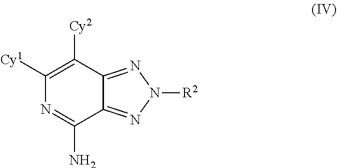
or a pharmaceutically acceptable salt thereof, wherein:
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and
each R^{2A} is independently selected from halo, C₁₋₆ alkyl, and C₁₋₆ haloalkyl. or a pharmaceutically acceptable salt thereof.

9. The method of claim 1, wherein the inhibitor of A2A/A2B is selected from:
3-(4-amino-2-(pyridin-2-ylmethyl)-7-(pyrimidin-4-yl)-2H-[1,2,3]triazolo[4,5-c]pyridin-6-yl)benzonitrile;
3-(4-amino-2-((3-fluoropyridin-2-yl)methyl)-7-(pyrimidin-4-yl)-2H-[1,2,3]triazolo[4,5-c]pyridin-6-yl)benzonitrile;
3-(4-amino-2-((3-fluoropyridin-2-yl)methyl)-7-(pyridin-4-yl)-2H-[1,2,3]triazolo[4,5-c]pyridin-6-yl)benzonitrile; and
3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile;
or a pharmaceutically acceptable salt of any of the aforementioned.

10. The method of claim 1, wherein the inhibitor of A2A/A2B is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof.

11. The method of claim 1, wherein the inhibitor of A2A/A2B is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof.

12. The method of claim 1, wherein the inhibitor of PD-1/PD-L1 is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.

13. The method of claim 1, wherein the inhibitor of PD-1/PD-L1 is pembrolizumab or atezolizumab.

14. (canceled)

15. The method of claim 1, wherein the inhibitor of PD-1/PD-L1 is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:
the VH CDR1 comprises the amino acid sequence SYWMN (SEQ ID NO:6);
the VH CDR2 comprises the amino acid sequence VIHPSDSETWLDQKFKD (SEQ ID NO:7); and
the VH CDR3 comprises the amino acid sequence EHYGTSPFAY (SEQ ID NO:8); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:9);
the VL CDR2 comprises the amino acid sequence AASNQGS (SEQ ID NO:10); and
the VL CDR3 comprises the amino acid sequence QQSKEVPYT (SEQ ID NO:11).

16. The method of claim 15, wherein the VH domain comprises the amino acid sequence set forth in SEQ ID NO:4 and the VL domain comprises the amino acid sequence set forth in SEQ ID NO:5.

17. The method of claim 15, wherein the antibody comprises a heavy chain and a light chain, and wherein the heavy chain comprises the amino acid sequence set forth in SEQ ID NO:2 and the light chain comprises the amino acid sequence set forth in SEQ ID NO:3.

18. The method of claim 15, wherein the antibody or antigen-binding fragment thereof that binds to human PD-1 is a humanized antibody.

19. The method of claim 1, wherein the inhibitor of human CD73 comprises:
(a) an antibody that binds to human CD73 and comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO:16);
the VH CDR2 comprises the amino acid sequence IYPGSGNT (SEQ ID NO:17); and
the VH CDR3 comprises the amino acid sequence ARYDYLGSSYGFDY (SEQ ID NO:18); and
comprising a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) an antibody that binds to human CD73 at an epitope within amino acids 40-53 of SEQ ID NO:70;
(c) an antibody that binds to human CD73 and competes for binding to human CD73 with an antibody that has a heavy chain comprising the amino acid sequence of SEQ ID NO:24 and a light chain comprising the amino acid sequence of SEQ ID NO:25;
(d) an antibody that binds to human CD73 and comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
an antibody comprising a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) an antibody that binds to human CD73 at an epitope within amino acids 386-399 and 470-489 of SEQ ID NO:70;
(f) an antibody that binds to human CD73 and competes for binding to human CD73 with an antibody that has a heavy chain comprising the amino acid sequence of SEQ ID NO:30 and a light chain comprising the amino acid sequence of SEQ ID NO:31;
(g) an antibody that binds to human CD73 and competes for binding to human CD73 with an antibody that has a heavy chain comprising the amino acid sequence of SEQ ID NO:33 and a light chain comprising the amino acid sequence of SEQ ID NO:31;
(h) an antibody selected from the group consisting of 11E1, Medi9447, CPI-006, and BMS-986179; or
(i) an inhibitor selected from the group consisting of CB-708 and AB680.

20. The method of claim 1, wherein the inhibitor of human CD73 is an antibody that binds to human CD73 and comprises:
a VH domain comprising VH CDR1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO:17); and
the VH CDR3 comprises the amino acid sequence ARYDYLGSSYGFDY (SEQ ID NO:18); and
a VL domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21).

21. The method of claim 1, wherein the inhibitor of human CD73 comprises an antibody that binds to human CD73 at an epitope within amino acids 40-53 of SEQ ID NO:70.

22. The method of claim 1, wherein the inhibitor of human CD73 comprises an antibody that binds to human CD73 and competes for binding to human CD73 with an antibody that has a heavy chain comprising the amino acid sequence of SEQ ID NO:24 and a light chain comprising the amino acid sequence of SEQ ID NO:25.

23. The method of claim 1, wherein the inhibitor of human CD73 comprises an antibody that binds to human CD73 and comprises:
a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39).

24. The method of claim 1, wherein the inhibitor of human CD73 comprises an antibody that binds to human CD73 at an epitope within amino acids 386-399 and 470-489 of SEQ ID NO:70.

25. The method of claim 1, wherein the inhibitor of human CD73 comprises an antibody that binds to human CD73 and competes for binding to human CD73 with an antibody that has a heavy chain comprising the amino acid sequence of SEQ ID NO:30 and a light chain comprising the amino acid sequence of SEQ ID NO:31.

26. The method of claim 1, wherein the inhibitor of human CD73 comprises an antibody that binds to human CD73 and competes for binding to human CD73 with an antibody that has a heavy chain comprising the amino acid sequence of SEQ ID NO:33 and a light chain comprising the amino acid sequence of SEQ ID NO:31.

27. The method of claim 1, wherein the inhibitor of human CD73 comprises an antibody selected from the group consisting of 11E1, Medi9447, CPI-006, and BMS-986179.

28. The method of claim 1, wherein the inhibitor of human CD73 is selected from the group consisting of CB-708 and AB680.

29. The method of claim 20, wherein the VH domain comprises the amino acid sequence set forth in SEQ ID NO:22 and the VL domain comprises the amino acid sequence set forth in SEQ ID NO:23.

30. The method of claim 20, wherein the antibody comprises a heavy chain and a light chain, and wherein the heavy chain comprises the amino acid sequence set forth in SEQ ID NO:24 and the light chain comprises the amino acid sequence set forth in SEQ ID NO:25.

31. The method of claim 23, wherein the VH domain comprises the amino acid sequence set forth in SEQ ID NO:62 and the VL domain comprises the amino acid sequence set forth in SEQ ID NO:61.

32. The method of claim 23, wherein the antibody comprises a heavy chain and a light chain, and wherein the heavy chain comprises the amino acid sequence set forth in SEQ ID NO:30 and the light chain comprises the amino acid sequence set forth in SEQ ID NO:31.

33. The method of claim 23, wherein the VH domain comprises the amino acid sequence set forth in SEQ ID NO:63 and the VL domain comprises the amino acid sequence set forth in SEQ ID NO:61.

34. The method of claim 23, wherein the antibody comprises a heavy chain and a light chain, and wherein the heavy chain comprises the amino acid sequence set forth in SEQ ID NO:33 and the light chain comprises the amino acid sequence set forth in SEQ ID NO:31.

35. The method of claim 1, wherein the cancer is selected from bladder cancer, breast cancer, cervical cancer, colon cancer, rectal cancer, colorectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, renal cell carcinoma, and Merkel cell carcinoma.

36. (canceled)

37. The method of claim 1, wherein the cancer is bladder cancer.

38. The method of claim 1, wherein the cancer is breast cancer.

39. A method of treating a cancer selected from bladder cancer, breast cancer, cervical cancer, colon cancer, rectal cancer, colorectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, renal cell carcinoma, and Merkel cell carcinoma in a subject, comprising administering to the subject:
(i) an inhibitor of A2A/A2B;
(ii) an inhibitor of PD-1/PD-L1, which is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:
the VH CDR1 comprises the amino acid sequence SYWMN (SEQ ID NO:6);
the VH CDR2 comprises the amino acid sequence VIHPSDSETWLDQKFKD (SEQ ID NO:7); and
the VH CDR3 comprises the amino acid sequence EHYGTSPFAY (SEQ ID NO:8); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:9);
the VL CDR2 comprises the amino acid sequence AASNQGS (SEQ ID NO:10); and
the VL CDR3 comprises the amino acid sequence QQSKEVPYT (SEQ ID NO:11); and
(iii) an antibody that binds to human CD73, wherein the antibody that binds to human CD73:
(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO:16);
the VH CDR2 comprises the amino acid sequence IYPGSGNT (SEQ ID NO: 17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

40-74. (canceled)

75. The method of claim 39, wherein the cancer is bladder cancer.

76. The method of claim 39, wherein the cancer is breast cancer.

77. A method of treating breast cancer in a subject, comprising administering to the subject:
(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof,
(ii) an inhibitor of PD-1/PD-L1, which is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:
the VH CDR1 comprises the amino acid sequence SYWMN (SEQ ID NO:6);
the VH CDR2 comprises the amino acid sequence VIHPSDSETWLDQKFKD (SEQ ID NO:7); and
the VH CDR3 comprises the amino acid sequence EHYGTSPFAY (SEQ ID NO:8); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:9);
the VL CDR2 comprises the amino acid sequence AASNQGS (SEQ ID NO:10); and
the VL CDR3 comprises the amino acid sequence QQSKEVPYT (SEQ ID NO:11); and
(iii) an antibody that binds to human CD73, wherein the antibody that binds to human CD73:
(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO: 17); and
the VH CDR3 comprises the amino acid sequence ARYDYLGSSYGFDY (SEQ ID NO:18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

78. A method of treating breast cancer in a subject, comprising administering to the subject:
(i) an inhibitor of A2A/A2B, which is 3-(8-amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof,
(ii) an inhibitor of PD-1/PD-L1, which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof, and
(iii) an antibody that binds to human CD73, wherein the antibody that binds to human CD73:
(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO: 17); and
the VH CDR3 comprises the amino acid sequence ARYDYLGSSYGFDY (SEQ ID NO: 18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

79-80. (canceled)

81. The method of claim 1, wherein the inhibitor of A2A/A2B is administered to the subject in a dosage of from about 0.1 mg to about 1000 mg on a free base basis.

82. The method of claim 1, wherein the A2A/A2B inhibitor is administered to the subject once-daily, every other day, or once-weekly.

83. The method of claim 1, wherein the inhibitor of A2A/A2B, inhibitor of PD-1/PD-L1, and inhibitor of human CD73 are administered simultaneously.

84. The method of claim 1, wherein the inhibitor of A2A/A2B, inhibitor of PD-1/PD-L1, and inhibitor of human CD73 are administered sequentially.

85. A method of treating cancer in a subject, comprising administering to the subject:
(i) an inhibitor of PD-1/PD-L1; and
(ii) an inhibitor of human CD73.

86. A method of treating a cancer selected from neck and head cancer, lung cancer, ovarian cancer, prostate cancer, breast cancer, bladder cancer, colorectal cancer, gastric cancer, gastroesophageal junction cancer, anal cancer, liver cancer, or pancreatic cancer in a subject, comprising administering to the subject:
(i) an inhibitor of PD-1/PD-L1, which is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:
the VH CDR1 comprises the amino acid sequence SYWMN (SEQ ID NO:6);
the VH CDR2 comprises the amino acid sequence VIHPSDSETWLDQKFKD (SEQ ID NO:7); and
the VH CDR3 comprises the amino acid sequence EHYGTSPFAY (SEQ ID NO:8); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:9);
the VL CDR2 comprises the amino acid sequence AASNQGS (SEQ ID NO:10); and
the VL CDR3 comprises the amino acid sequence QQSKEVPYT (SEQ ID NO:11); and
(ii) an antibody that binds to human CD73, wherein the antibody that binds to human CD73:
(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO: 17); and
the VH CDR3 comprises the amino acid sequence ARYDYLGSSYGFDY (SEQ ID NO: 18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

87. A method of treating a cancer selected from squamous cell carcinoma of the neck and head (SCCNH), non-small cell lung cancer (NSCLC), ovarian cancer, castration-resistant prostate cancer (CRPC), triple-negative breast cancer (TNBC), bladder cancer, metastatic colorectal cancer (mCRC), pancreatic ductal adenocarcinoma (PDAC), gastric/gastroesophageal junction (GEJ) cancer, hepatocellular carcinoma (HCC), and squamous carcinoma of the anal canal (SCAC) in a subject, comprising administering to the subject:
(i) an inhibitor of PD-1/PD-L1, which is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:
the VH CDR1 comprises the amino acid sequence SYWMN (SEQ ID NO:6);
the VH CDR2 comprises the amino acid sequence VIHPSDSETWLDQKFKD (SEQ ID NO:7); and
the VH CDR3 comprises the amino acid sequence EHYGTSPFAY (SEQ ID NO:8); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:9);
the VL CDR2 comprises the amino acid sequence AASNQGS (SEQ ID NO:10); and
the VL CDR3 comprises the amino acid sequence QQSKEVPYT (SEQ ID NO:11); and
(ii) an antibody that binds to human CD73, wherein the antibody that binds to human CD73:
(a) comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 16);
the VH CDR2 comprises the amino acid sequence IYPGSGNT (SEQ ID NO: 17); and
the VH CDR3 comprises the amino acid sequence ARYDYLGSSYGFDY (SEQ ID NO: 18); and
comprises a variable light (VL) domain comprising VL CDR1, VL CDR2, and VL CDR3, wherein:
the VL CDR1 comprises the amino acid sequence QDVSTA (SEQ ID NO:19);
the VL CDR2 comprises the amino acid sequence SAS (SEQ ID NO:20); and
the VL CDR3 comprises the amino acid sequence QQHYNTPYT (SEQ ID NO:21);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

88. A method of treating a cancer selected from neck and head cancer, lung cancer, ovarian cancer, prostate cancer, breast cancer, bladder cancer, colorectal cancer, gastric cancer, gastroesophageal junction cancer, anal cancer, liver cancer, and pancreatic cancer in a subject, comprising administering to the subject:
(i) an inhibitor of PD-1/PD-L1, which is retifanlimab; and
(ii) an antibody that binds to human CD73, which is ANTIBODY Y.

89. A method of treating a cancer selected from squamous cell carcinoma of the neck and head (SCCNH), non-small cell lung cancer (NSCLC), ovarian cancer, castration-resistant prostate cancer (CRPC), triple-negative breast cancer (TNBC), bladder cancer, metastatic colorectal cancer (mCRC), and pancreatic cancer in a subject, comprising administering to the subject:
(i) an inhibitor of PD-1/PD-L1, which is retifanlimab; and
(ii) an antibody that binds to human CD73, which is ANTIBODY Y.


SEQUENCE LISTING (101 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 445 aa, Artificial Sequence
  Description of Artificial Sequence: Synthetic polypeptide
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 218 aa, Artificial Sequence
  Description of Artificial Sequence: Synthetic polypeptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 119 aa, Artificial Sequence
  Description of Artificial Sequence: Synthetic polypeptide
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 5 - protein, 111 aa, Artificial Sequence
  Description of Artificial Sequence: Synthetic polypeptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 6 - protein, 5 aa, Artificial Sequence
  Description of Artificial Sequence: Synthetic peptide
SYWMN

SEQ ID NO: 7 - protein, 17 aa, Artificial Sequence
  Description of Artificial Sequence: Synthetic peptide
VIHPSDSETWLDQKFKD

SEQ ID NO: 8 - protein, 10 aa, Artificial Sequence
  Description of Artificial Sequence: Synthetic peptide
EHYGTSPFAY

SEQ ID NO: 9 - protein, 16 aa, Artificial Sequence
  Description of Artificial Sequence: Synthetic peptide
RASESVDNYGMSFMNW

SEQ ID NO: 10 - protein, 7 aa, Artificial Sequence
  Description of Artificial Sequence: Synthetic peptide
AASNQGS

SEQ ID NO: 11 - protein, 9 aa, Artificial Sequence
  Description of Artificial Sequence: Synthetic peptide
QQSKEVPYT

SEQ ID NO: 12-15 - skipped (4 entries)

SEQ ID NO: 16 - protein, 8 aa, Artificial Sequence
  Description of Artificial Sequence: Synthetic peptide
GYTFTSYG

SEQ ID NO: 17 - protein, 8 aa, Artificial Sequence
  Description of Artificial Sequence: Synthetic peptide
IYPGSGNT

SEQ ID NO: 18 - protein, 14 aa, Artificial Sequence
  Description of Artificial Sequence: Synthetic peptide
ARYDYLGSSYGFDY

SEQ ID NO: 19 - protein, 6 aa, Artificial Sequence
  Description of Artificial Sequence: Synthetic peptide
QDVSTA

SEQ ID NO: 20 - protein, 3 aa, Artificial Sequence
  Description of Artificial Sequence: Synthetic peptide
SAS

SEQ ID NO: 21 - protein, 9 aa, Artificial Sequence
  Description of Artificial Sequence: Synthetic peptide
QQHYNTPYT

SEQ ID NO: 22 - protein, 121 aa, Artificial Sequence
  Description of Artificial Sequence: Synthetic polypeptide
EVQLVQSGAEVKKPGESLKISCKGSGYTFTSYGLSWVRQMPGKGLEWMGEIYPGSGNTYYNEKFKGQVTISADKSISTAYLQWSSLKASDTAMYYCARYDYLGSSYGFDYWGAGTTVTVSS

SEQ ID NO: 23 - protein, 107 aa, Artificial Sequence
  Description of Artificial Sequence: Synthetic polypeptide
DIVMTQSPDSLAVSLGERATINCKASQDVSTAVAWYQQKPGQPPKLLIYSASYRYSGVPDRFSGSGSGTDFTLTISSLQAEDVAVYYCQQHYNTPYTFGGGTKLEIK

SEQ ID NO: 24 - protein, 450 aa, Artificial Sequence
  Description of Artificial Sequence: Synthetic polypeptide
EVQLVQSGAEVKKPGESLKISCKGSGYTFTSYGLSWVRQMPGKGLEWMGEIYPGSGNTYYNEKFKGQVTISADKSISTAYLQWSSLKASDTAMYYCARYDYLGSSYGFDYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 214 aa, Artificial Sequence
  Description of Artificial Sequence: Synthetic polypeptide
DIVMTQSPDSLAVSLGERATINCKASQDVSTAVAWYQQKPGQPPKLLIYSASYRYSGVPDRFSGSGSGTDFTLTISSLQAEDVAVYYCQQHYNTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26-29 - skipped (4 entries)

SEQ ID NO: 30 - protein, 446 aa, Artificial Sequence
  Description of Artificial Sequence: Synthetic polypeptide
EVQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 212 aa, Artificial Sequence
  Description of Artificial Sequence: Synthetic polypeptide
IQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - skipped

SEQ ID NO: 33 - protein, 446 aa, Artificial Sequence
  Description of Artificial Sequence: Synthetic polypeptide
EVQLVESGGGLVQPGGSLRLSCAASGFTFSSYDMHWVRQAPGKGLEWVAVMSYEGSNKYYADSVKGRFTISRDNSKNALYLQMNSLRAEDTAVYYCATEIAAKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 8 aa, Artificial Sequence
  Description of Artificial Sequence: Synthetic peptide
GFTFSSYD

SEQ ID NO: 35 - protein, 8 aa, Artificial Sequence
  Description of Artificial Sequence: Synthetic peptide
MSYDGSNK

SEQ ID NO: 36 - protein, 10 aa, Artificial Sequence
  Description of Artificial Sequence: Synthetic peptide
ATEIAAKGDY

SEQ ID NO: 37 - protein, 6 aa, Artificial Sequence
  Description of Artificial Sequence: Synthetic peptide
QGISNY

SEQ ID NO: 38 - protein, 3 aa, Artificial Sequence
  Description of Artificial Sequence: Synthetic peptide
AAS

SEQ ID NO: 39 - protein, 8 aa, Artificial Sequence
  Description of Artificial Sequence: Synthetic peptide
QQSYSTPH

SEQ ID NO: 40 - protein, 8 aa, Artificial Sequence
  Description of Artificial Sequence: Synthetic peptide
MSYEGSNK

SEQ ID NO: 41-59 - skipped (19 entries)

SEQ ID NO: 60 - protein, 116 aa, Artificial Sequence
  Description of Artificial Sequence: Synthetic polypeptide
VQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSS

SEQ ID NO: 61 - protein, 105 aa, Artificial Sequence
  Description of Artificial Sequence: Synthetic polypeptide
IQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIK

SEQ ID NO: 62 - protein, 117 aa, Artificial Sequence
  Description of Artificial Sequence: Synthetic polypeptide
EVQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSS

SEQ ID NO: 63 - protein, 117 aa, Artificial Sequence
  Description of Artificial Sequence: Synthetic polypeptide
EVQLVESGGGLVQPGGSLRLSCAASGFTFSSYDMHWVRQAPGKGLEWVAVMSYEGSNKYYADSVKGRFTISRDNSKNALYLQMNSLRAEDTAVYYCATEIAAKGDYWGQGTLVTVSS

SEQ ID NO: 64-65 - skipped (2 entries)

SEQ ID NO: 66 - protein, 446 aa, Artificial Sequence
  Description of Artificial Sequence: Synthetic polypeptide
VQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67-69 - skipped (3 entries)

SEQ ID NO: 70 - protein, 523 aa, Homo sapiens
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFS

SEQ ID NO: 71 - protein, 523 aa, Mus musculus
WELTILHTNDVHSRLEQTSDDSTKCLNASLCVGGVARLFTKVQQIRKEEPNVLFLDAGDQYQGTIWFTVYKGLEVAHFMNILGYDAMALGNHEFDNGVEGLIDPLLRNVKFPILSANIKARGPLAHQISGLFLPSKVLSVGGEVVGIVGYTSKETPFLSNPGTNLVFEDEISALQPEVDKLKTLNVNKIIALGHSGFEMDKLIAQKVRGVDIVVGGHSNTFLYTGNPPSKEVPAGKYPFIVTADDGRQVPVVQAYAFGKYLGYLKVEFDDKGNVITSYGNPILLNSSIPEDATIKADINQWRIKLDNYSTQELGRTIVYLDGSTQTCRFRECNMGNLICDAMINNNLRHPDEMFWNHVSMCIVNGGGIRSPIDEKNNGTITWENLAAVLPFGGTFDLVQLKGSTLKKAFEHSVHRYGQSTGEFLQVGGIHVVYDINRKPWNRVVQLEVLCTKCRVPIYEPLEMDKVYKVTLPSYLANGGDGFQMIKDELLKHDSGDQDISVVSEYISKMKVVYPAVEGRIKFS

SEQ ID NO: 72 - protein, 523 aa, Macaca sp.
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFETDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHADEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEIYKVILPNFLANGGDGFQMIKDELLRHDSGDQDINVVSTYISKMKVIYPAVEGRIKFS

SEQ ID NO: 73-75 - skipped (3 entries)

SEQ ID NO: 76 - protein, 14 aa, Homo sapiens
TKVQQIRRAEPNVL

SEQ ID NO: 77 - skipped

SEQ ID NO: 78 - protein, 14 aa, Homo sapiens
AAVLPFGGTFDLVQ

SEQ ID NO: 79 - protein, 20 aa, Homo sapiens
ILPNFLANGGDGFQMIKDEL

SEQ ID NO: 80-93 - skipped (14 entries)

SEQ ID NO: 94 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 95 - protein, 332 aa, Homo sapiens
MLLETQDALYVALELVIAALSVAGNVLVCAAVGTANTLQTPTNYFLVSLAAADVAVGLFAIPFAITISLGFCTDFYGCLFLACFVLVLTQSSIFSLLAVAVDRYLAICVPLRYKSLVTGTRARGVIAVLWVLAFGIGLTPFLGWNSKDSATNNCTEPWDGTTNESCCLVKCLFENVVPMSYMVYFNFFGCVLPPLLIMLVIYIKIFLVACRQLQRTELMDHSRTTLQREIHAAKSLAMIVGIFALCWLPVHAVNCVTLFQPAQGKNKPKWAMNMAILLSHANSVVNPIVYAYRNRDFRYTFHKIISRYLLCQADVKSGNGQAGVQPALGVGL

SEQ ID NO: 96 - protein, 117 aa, Artificial Sequence
  Description of Artificial Sequence: Synthetic polypeptide
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 97 - protein, 110 aa, Artificial Sequence
  Description of Artificial Sequence: Synthetic polypeptide
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVL

SEQ ID NO: 98-99 - skipped (2 entries)

SEQ ID NO: 100 - protein, 120 aa, Artificial Sequence
  Description of Artificial Sequence: Synthetic polypeptide
EIQLQQSGPELVKPGASVKVSCKASGYAFTSYNMYWVKQSHGKSLEWIGYIDPYNGGTSYNQKFKGKATLTVDKSSSTAYMHLNSLTSEDSAVYYCARGYGNYKAWFAYWGQGTLVTVSA

SEQ ID NO: 101 - protein, 106 aa, Artificial Sequence
  Description of Artificial Sequence: Synthetic polypeptide
DAVMTQTPKFLLVSAGDRVTITCKASQSVTNDVAWYQQKPGQSPKLLIYYASNRYTGVPDRFTGSGYGTDFTFTISTVQAEDLAVYFCQQDYSSLTFGAGTKLELK